(19) United States
(12) Patent Application Publication
(10) Pub. No.: US 2017/0344699 A1
(43) Pub. Date: Nov. 30, 2017

(54) WATER-SOLUBLE MEMBRANE PROTEINS AND METHODS FOR THE PREPARATION AND USE THEREOF

(71) Applicant: Massachusetts Institute of Technology, Cambridge, MA (US)

(72) Inventors: Shuguang Zhang, Lexington, MA (US); Fei Tao, Cambridge, MA (US)

(73) Assignee: Massachusetts Institute of Technology, Cambridge, MA (US)

(21) Appl. No.: 15/581,495

(22) Filed: Apr. 28, 2017

Related U.S. Application Data
(63) Continuation of application No. 14/669,753, filed on Mar. 26, 2015, now abandoned.
(60) Provisional application No. 62/117,550, filed on Feb. 18, 2015.
(60) Provisional application No. 61/993,783, filed on May 15, 2014.
(60) Provisional application No. 61/971,388, filed on Mar. 27, 2014.

(51) Int. Cl. G06F 19/12; G06F 19/16; C07K 14/72; G06F 19/00; C07K 14/705

(52) U.S. Cl. CPC G06F 19/12; G06F 19/706; C07K 14/705; C07K 14/723; G06F 19/16

(57) ABSTRACT

The present invention is directed to water-soluble membrane proteins, methods for the preparation thereof and methods of use thereof.

### RELATED APPLICATIONS

This application relates to U.S. Provisional Application No. 61/971,388, filed on Mar. 27, 2014. The entire teaching of the application is incorporated herein by reference.

### BACKGROUND OF THE INVENTION

Membrane proteins play vital roles in all living systems. Approximately ˜30% of all genes in almost all sequenced genomes code for membrane proteins. However, our detailed understanding of their structure and function lags far behind that of soluble proteins. As of March 2015, there are over 100,000 structures in the Protein Data Bank (http://www.rcsb.org/pdb/home/home.do). However, there are only 945 membrane protein structures with 530 unique structures including 28 G-protein coupled receptors and no tetraspanin membrane proteins. http://blanco.biomol.uci.edu/mpstruc/

There are several bottlenecks in elucidating the structure and function of membrane receptors and their recognition and ligand-binding properties although they are of great interest. The most critical and challenging task is that it is extremely difficult to produce milligram quantities of soluble and stable receptors. Inexpensive large-scale production methods are desperately needed, and have thus been the focus of extensive research. It is only possible to conduct detailed structural studies once these preliminary obstacles have been surmounted.

Zhang et al., (U.S. Pat. No. 8,637,452), incorporated herein by reference, describes an improved process for water solubilizing GPCRs wherein certain hydrophobic amino acids located in the transmembrane regions were substituted by polar amino acids. However, the process is labor-intensive. Further, while the modified transmembrane regions met the water-soluble criteria, improvements in water solubility and ligand binding are desired. Therefore, there is a need in the art for improved methods of studying G-protein coupled receptors.

### SUMMARY OF THE INVENTION

The present invention is directed to a method of designing, selecting and/or producing water-soluble membrane proteins and peptides, peptides (and transmembrane domains) designed, selected or produced therefrom, compositions comprising said peptides, and methods of use thereof. In particular, the method relates to a process for designing a library of water soluble membrane peptides, such as GPCR variants and tetraspanin membrane proteins, using the “QTY Principle,” changing the water-insoluble amino acids (Leu, Ile, Val and Phe, or the simple letter code L, I, V, F) into water-soluble, non-ionic amino acids (Gln, Thr and Tyr, or the simple letter code Q, T, Y). Furthermore, two additional non-ionic amino acids Asn (N) and Ser (S) may also be used for the substitution for L, I and V but not for F. In the embodiments discussed below, it is to be understood that Asn (N) and Ser (S) are envisioned as being substitutable for Q and T (as a variant is described) or L, I or V (as a native protein is described). For the purposes of brevity, however, the application does not explicitly state these alternative embodiments.

The invention encompasses a modified, synthetic, and/or non-naturally occurring, α-helical domain(s) and water-soluble polypeptide (e.g., “sGPCR”) comprising such modified α-helical domain(s), wherein the modified α-helical domain(s) comprise an amino acid sequence in which a plurality of hydrophobic amino acid residues (L, I V, F) within a α-helical domain of a native membrane protein are replaced with hydrophilic, non-ionic amino acid residues (Q, T, T, Y, respectively, or “Q, T, Y”) and/or N and S. The invention also encompasses a method of preparing a water-soluble polypeptide comprising replacing a plurality of hydrophobic amino acid residues (L, I V, F) within the α-helical domain(s) of a native membrane protein with hydrophilic, non-ionic amino acid residues (Q, T, Y). The invention additionally encompasses a polypeptide prepared by replacing a plurality of hydrophobic amino acid residues (L, I V, F) within the α-helical domain of a native membrane protein with hydrophilic, non-ionic amino acid residues (Q, T, Y., respectively). The variant can be characterized by the name of the parent or native protein (e.g., CXCR4) followed by the abbreviation “QTY” (e.g., CXCR4-QTY).

The invention further encompasses a method of treatment for a disorder or disease that is mediated by the activity a membrane protein in a subject in need thereof, comprising administering to said subject an effective amount of a water-soluble polypeptide described herein.

In certain aspects, the water-soluble polypeptide retains the ligand-binding activity of the membrane protein. Examples of disorders and diseases that can be treated by administering a water-soluble peptide of the invention include, but are not limited to, cancer (such as, small cell lung cancer, melanoma, triple negative breast cancer), Parkinson's disease, cardiovascular disease, hypertension, and bronchial asthma.

The invention also encompasses a pharmaceutical composition comprising a water-soluble polypeptide of the invention and pharmaceutically acceptable carrier or diluent.

In some aspects, the α-helical domain is one of 7-transmembrane α-helical domains in a native membrane protein is a G-protein coupled receptor (GPCR). In some aspects of this embodiment, the GPCR is selected from the group comprising purinergic receptors (P2Y₁, P2Y₂, P2Y₄, P2Y₆), M₁ and M₃ muscarinic acetylcholine receptors, receptors for thrombin [protease-activated receptor (PAR)-1, PAR-2], thromboxane (TXA₂), sphingosine 1-phosphate (S1P₂, S1P₃, S1P₄ and S1P₅), lysophosphatidic acid (LPA₁, LPA₂, LPA₃), angiotensin II (AT₁), serotonin (5-HT_{2c} and 5-HT₄), somatostatin (sst₅), endothelin (ET_{A} and ET_{B}), cholecystokinin (CCK₁), V₁ₐ vasopressin receptors, D₅ dopamine receptors, fMLP formyl peptide receptors, GAL₂ galanin receptors, EP₃ prostanoid receptors, A₁ adenosine receptors, α₁ adrenergic receptors, BB₂ bombesin receptors, B₂ bradykinin receptors, calcium-sensing receptors, chemokine receptors, KSHV-ORF74 chemokine receptors, NK₁ tachykinin receptors, thyroid-stimulating hormone (TSH) receptors, protease-activated receptors, neuropeptide receptors, adenosine A2B receptors, P2Y purinoceptors, metabolic glutamate receptors, GRK5, GPCR-30, and CXCR4. In yet an additional embodiment, the native membrane protein or membrane protein is an integral membrane protein. In a further aspect, the native membrane protein is a mammalian protein. The proteins of the invention are preferably human. For the purposes of being concise, references to specific GPCR proteins (e.g., CXCR4) are intended to refer to both mammalian, generally, and, in the alternative, human, specifically. In other embodiments, the α-helical domain is one of 7-transmembrane α-helical domains in a G-protein coupled receptor (GPCR) variant modified, for example, in the extracellular or intracellular loops to improve or alter ligand binding, as described elsewhere in the literature. For the purposes of this invention, the word “native” is intended to refer to the protein (or α-helical domain) prior to water solubilization in accordance with the methods described herein.

In another aspect of the invention the membrane protein can be a tetraspanin membrane protein characterized by 4 transmembrane alpha-helices. Approximately 54 human tetraspanin membrane proteins have been reviewed and annotated. Many are known to mediate cellular signal transduction events that play a critical role in regulation of cell development, activation, growth and motility, For example, CD81 receptor plays a critical role as the receptor for Hepatitis C virua entry and plasmodium infection. CD81 gene is localized in the tumor-suppressor gene region and can be a candidate for mediating cancer malignancies. CD151 is involved in enhanced cell motility, invasion and metastasis of cancer cells. Expression of CD63 correlates with the invasiveness of ovarian cancer. Characteristic of a tetraspanin membrane protein is a Cysteine-cysteine-glycine motif in the second, or large, extracellular loop.

The hydrophilic residues (which replace one or more hydrophobic residues in the α-helical domain of a native membrane protein) are selected from the group consisting of glutamine (Q), threonine (T), tyrosine (Y) and any combination thereof. In additional aspects, the hydrophobic residues selected from leucine (L), isoleucine (I), valine (V) and phenylalanine (F) are replaced. Specifically, the phenylalanine residues of the α-helical domain of the protein are replaced with tyrosine; the isoleucine and/or valine residues of the α-helical domain of the protein are replaced with threonine; and/or the leucine residues of the α-helical domain of the protein are replaced with glutamine.

Preferred water-soluble polypeptides of the invention possess the ability to bind the ligand which normally binds to the wild type or native membrane protein. In preferred embodiments, the amino acids within potential ligand binding sites of the native membrane protein are not replaced and/or the sequences of the extracellular and/or intracellular domains of the native membrane polypeptide are identical.

In yet an additional embodiment, the invention encompasses a cell transfected with a water-soluble peptide comprising a modified α-helical domain. In certain embodiments, the cell is an animal cell (e.g., mammalian, insect, avian, fish, reptile, amphibian, or other cell), yeast or a bacterial cell.

The invention also includes a computer implemented method performed on a computer system, the method comprising one or more of the methods (or steps thereof) as described herein. Computer systems including a non-transient computer readable medium having computer-executable instructions stored thereon, the computer-executable instructions when executed by the computer system causing the computer system to perform the methods and non-transient computer readable media having computer-executable instructions stored thereon, the computer-executable instructions when executed by the computer system causing the computer system to perform the methods dare contemplated as well. Additionally, computer systems comprising a memory and at least one processor coupled to the memory, the processor being configured to perform the methods described herein are contemplated.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIGS. 1A-1D is the general illustration for the QTY Code that systematically substitutes the hydrophobic amino acids L, I, V and F to Q, T, T, Y, respectively, FIG. 1A). The molecular shapes of amino acids leucine and glutamine are similar; likewise molecular shapes of isoleucine and valine are similar to threonine; and molecular shapes of phenylalanine and tyrosine are similar. Leucine, isoleucine, valine and phenylalanine are hydrophobic and cannot bind with water molecules. In contrast, glutamine can bind with 4 water molecules, 2 hydrogen donors and 2 hydrogen acceptors; the —OH group on threonine and tyrosine can bind to 3 water molecules, 1 hydrogen donor and 2 acceptors. FIG. 1B is a side view of an alpha helix. After applying the QTY Code of systematic amino acid changes, the alpha helix become water-soluble. FIG. 1C) Top view of an alpha helix: the helix on the left is the natural membrane helix with mostly hydrophobic amino acids, the helix on the right is after applying QTY Code. The helix now has most hydrophilic amino acids (FIG. 1D). Before QTY Code, the GPCR membrane proteins are surrounded by hydrophobic lipid molecules to embed them inside the lipid membrane (left panel). After applying QTY Code, the GPCR membrane proteins become water-soluble and no long need detergent to surround it for stabilization.

FIG. 2 is the TMHMM prediction for the transmembrane domain regions for CXCR4. There are distinctive 7 hydrophobic transmembrane segments.

FIG. 3 illustrates the predicted alpha helical wheel structure of the fully modified of TM1 domain of CXCR4. The natural helix (left panel) and QTY Code modified helix (right panel).

FIG. 4 is an illustration of the TMHMM program output for CXCR4. There are no distinctive 7 hydrophobic transmembrane segments visible anymore.

FIGS. 5, 6, 7 and 8 are sequence alignments of the wild type proteins and QTY variants of CXCR4, CXCR3, CCR3 and CCR5, respectively. QTY Code is only applied to the 7 hydrophobic transmembrane segments, but not the extracellular and intracellular segments.

FIG. 9 is a flowchart of the process.

FIG. 10 is an illustration of the computer systems of the invention.

### DETAILED DESCRIPTION OF THE INVENTION

A description of preferred embodiments of the invention follows.

The words “a” or “an” are meant to encompass one or more, unless otherwise specified.

In some aspects, the invention is directed to the use of the QTY (Glutamine, threonine and tyrosine) replacement (or “Code”) method (or “principle”) to change the 7-transmembrane α-helix hydrophobic residues leucine (L), isoleucine (I), valine (V), and phenylalanine (F) of a native protein to the hydrophilic residues glutamine (Q), threonine (T) and tyrosine (Y), or alternatively, as described above, Asn (N) and Ser (S) for L, I and/or V. This invention can convert a water insoluble, native membrane protein to a water-soluble counterpart.

The invention includes a process for designing water-soluble peptides. The process is described in terms of GPCR proteins as an example, with specificity in the first instance to human CCR3, CCR5, CXCR4, and CX3CR1.

GPCRs typically have 7-transmembrane alpha-helices (7TM) and 8 loops. These transmembrane segments are called TM1, TM2, TM3, TM4, TM5, TM6 and TM7. The 8 non-transmembrane loops are divided into 4 extracellular loops EL1, EL2, EL3, EL4 and 4 intracellular loops, IL1, IL2, IL3, IL4, thus total 8 loops. We can therefore divide a GPCR protein into 15 fragments based on the transmembrane and non-transmembrane features. In broad terms, the process comprises all, or substantially all, the steps:
(1) identifying a first transmembrane region by predicting an alpha-helical structure of a protein;
(2) modifying a plurality of hydrophobic amino acids via the QTY Code, as defined herein to obtain a modified first transmembrane sequence;
(3) scoring the propensity of the alpha-helical structure of the first modified transmembrane sequence of (2) to arrive at a structure score;
(4) scoring the water solubility prediction of the first modified transmembrane sequence of (2) to arrive at a solubility score;
(5) repeating steps (2) through (4) to arrive at a first library of putative water soluble first modified transmembrane variants;
(6) comparing the structure scores and solubility scores of each putative water soluble first modified transmembrane variants in the first library and, preferably ranking the putative water soluble first modified transmembrane variants using said structure scores and solubility scores;
(7) selecting a plurality of putative water soluble first modified transmembrane variants (wherein the plurality is the integer, H, or preferably less than 10, 9, 8, 7, 6, 5 or 4) to arrive at a second library of putative water soluble first modified transmembrane variants;
(8) repeating steps (1) through (7) for a second, third, fourth, fifth, sixth, seventh or, preferably, all transmembrane regions of the protein (the sum of the transmembrane regions modified by the method being the integer n);
(9) identifying the amino acid sequences of the protein which are not included in any transmembrane region modified in steps (1) through (8), and including any extracellular or intracellular domain of the protein; and
(10) identifying a nucleic acid sequence for each putative water soluble modified transmembrane variant and each amino acid sequence identified in step (9).

Using the nucleic acid sequences identified in the above process, nucleic acid sequences for each putative water-soluble modified transmembrane variant and each non-transmembrane domains (including the extracellular and intracellular domains) can be generated and combinatorially expressed to design a library of up to Hⁿ putative water-soluble protein variants. For example, where H is 8 and n is 7, a library of approximately 2 million water-soluble protein variants can be designed.

The method provides for “scoring” the domains' including the propensity to form an alpha helix and water solubility prediction. As one of ordinary skill in the art would appreciate, the domains having different sequences will likely predict different water solubilities and propensities for alpha helical formation. One can assign “a score” to a specific predicted water solubility or range of solubilities, propensity to form alpha helical structure or range of propensities. The score can be qualitative (0,1) where 0 can represent, for example, a domain with an unacceptable predicted water solubility and 1 can represent, for example, a domain with an acceptable predicted water solubility. Or, the score can be assessed on a scale, for example, between 1 and 10 establishing characterizing increasing degrees of water solubility. Or, the score can be quantitative, such as in describing the predicted solubility in terms of mg/ml. Upon assessing a score to each domain, the domain variants can be readily compared (or ranked) by one or, preferably, both of the scores to select domain variants that will likely be both water soluble and form alpha helices.

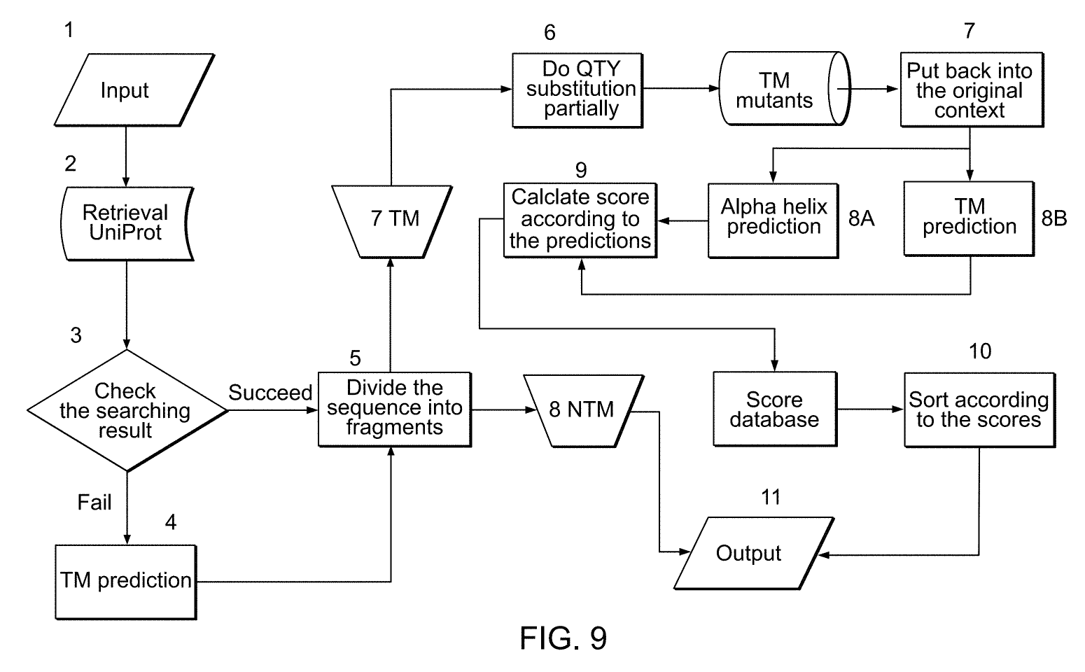

In a preferred embodiment, the process of designing the transmembrane regions is performed on a computer system, using the process described in FIG. 9.

### Step-by-Step Description:

1: In step 1, a computer interface of a computer system receives a protein sequence, selected for analysis, and data descriptive of the protein (e.g., the sequence) entered, uploaded or inputted through a computer interface of a computer system. The data entered can be a protein name, a database reference, or a protein sequence. For example, the protein sequence can be uploaded through a computer interface.

2: In step 2, additional data about the protein can be identified, determined, obtained and/or entered, including its name or sequence and entered via the computer interface. One source to obtain protein data is a database named UniProt (http://www.uniprot.org/). Alternatively, the method of the invention can store data relating to the protein, or related sequences to the protein, for later retrieval by the user in this step. In embodiments, the program can prompt the user to select a database or file for retrieving additional data (e.g., sequence data) relating to the protein selected for analysis.

3: In step 3, the user can enter, upload, or obtain data identifying the transmembrane regions. For example, the user can be prompted to obtain the data from a public source, such as from UniProt. The information can be collected from the database for use in Step 5.

4: Alternatively or additionally, the transmembrane region can be predicted by the method. Transmembrane regions are generally characterized by an alpha helical conformation. Transmembrane helix prediction can be predicted using a software package named TMHMM 2.0 (TransMembrane prediction using Hidden Markov Models), developed by Center for Biological Sequence Analysis (http://www.cbs.dtu.dk/services/TMHMM/). The current version of the software has some problems on peak finding and sometimes fails to find 7-TM regions for a GPCR. Therefore, in a preferred embodiment, a modified version of the program is used, wherein the peak searching method execute by the computer system introduces a dynamic baseline. Here, if the 7-TMs using the initial baseline value are not found, the baseline can be changed to a lower value. For example, the default baseline is 0.2. To identify a seventh transmembrane region, one can set the baseline value to 0.1. If more than 7-TM is found, the baseline can be changed to a higher value, such as 0.15. For example, when the CCR-2 amino acid sequence was subjected to the TMHMM 2.0 software, only 6 transmembrane regions were identified. When the TMHMM 2.0 baseline value was set to 0.07, 7 transmembrane regions were identified.

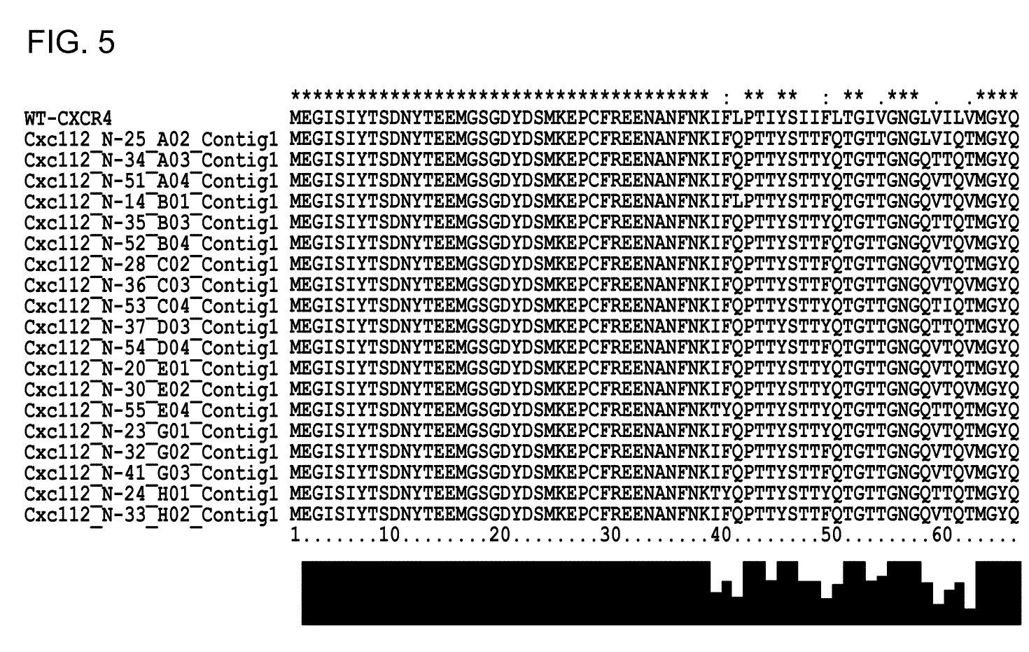

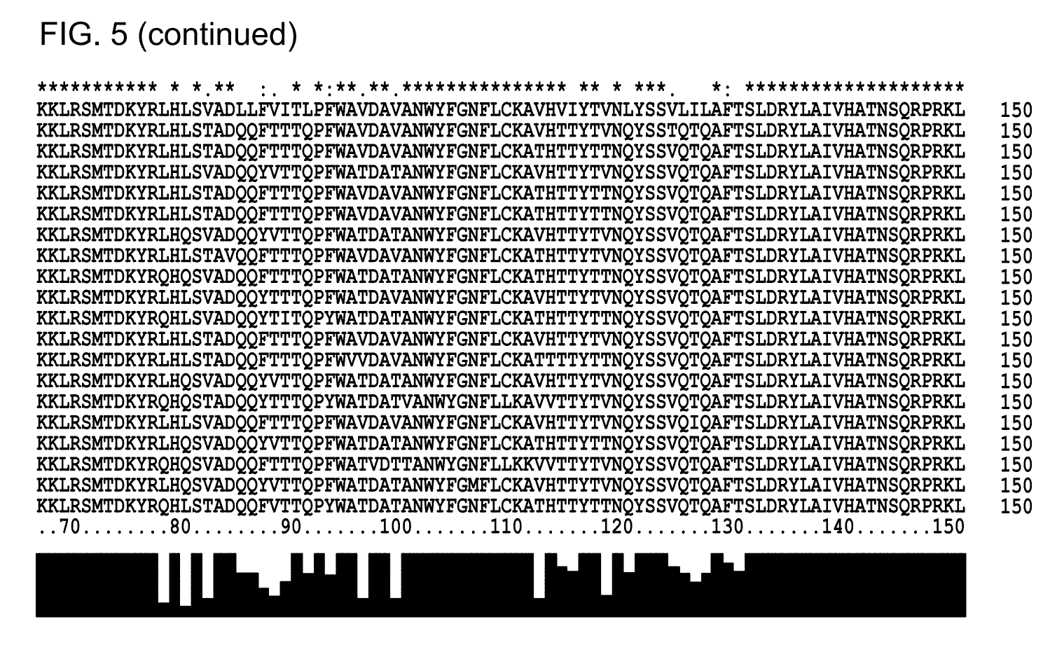

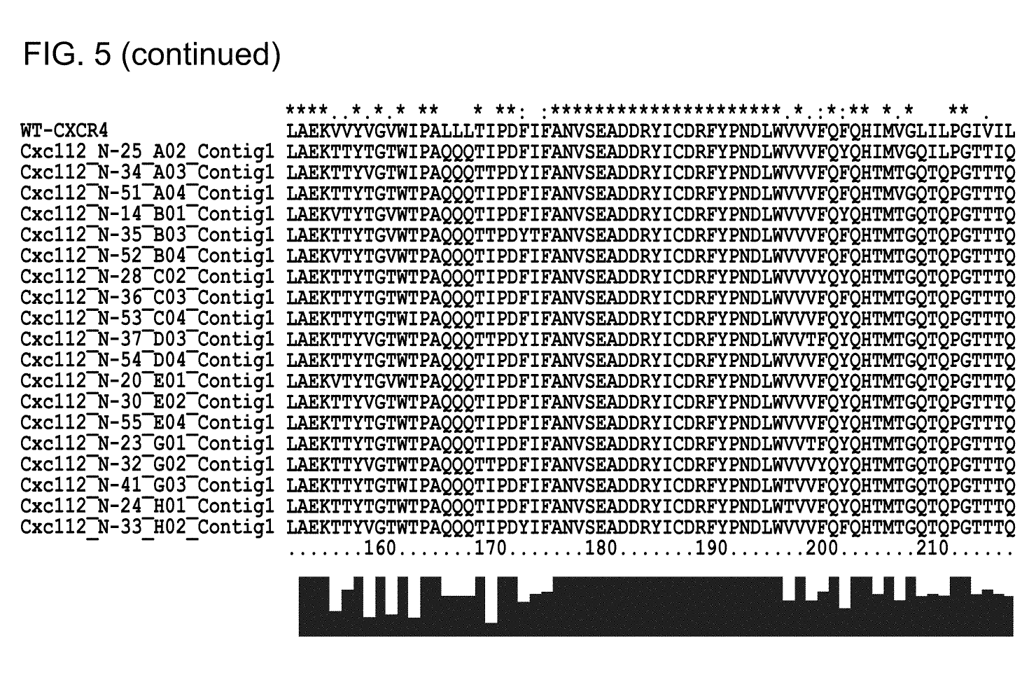

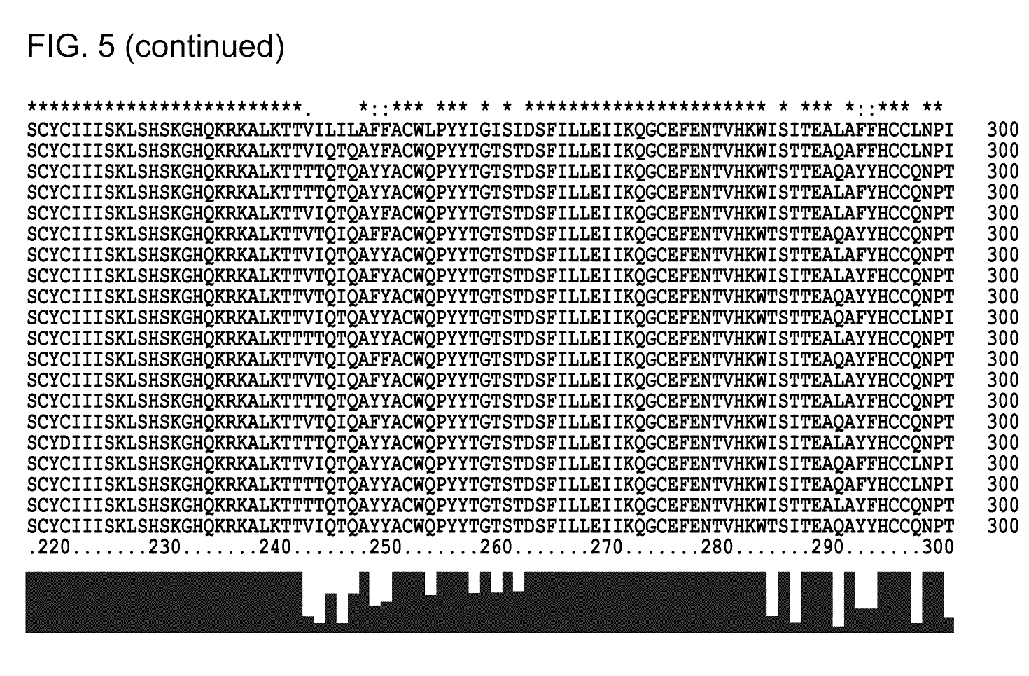

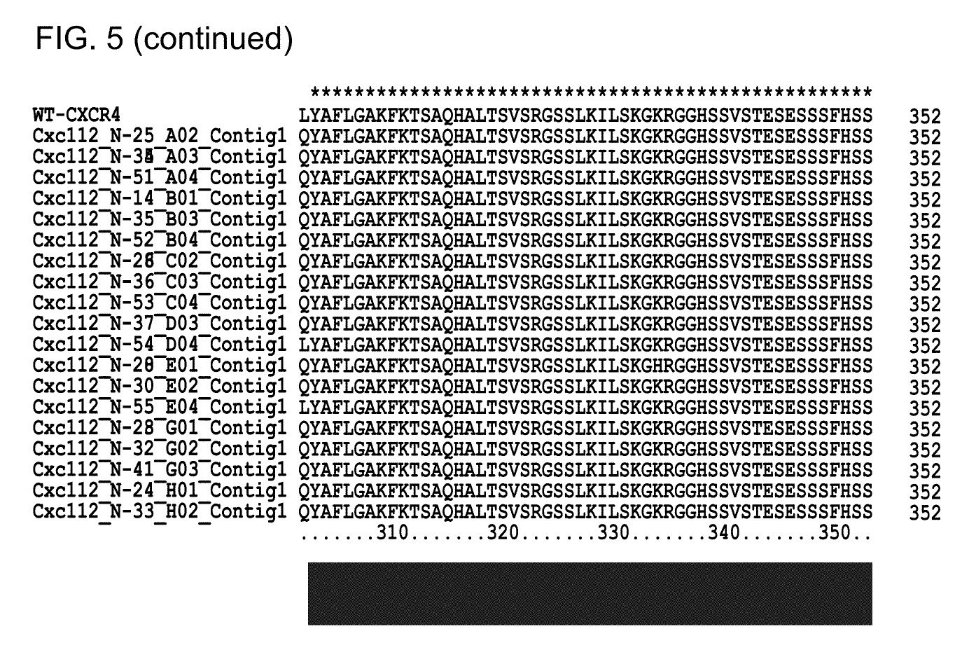

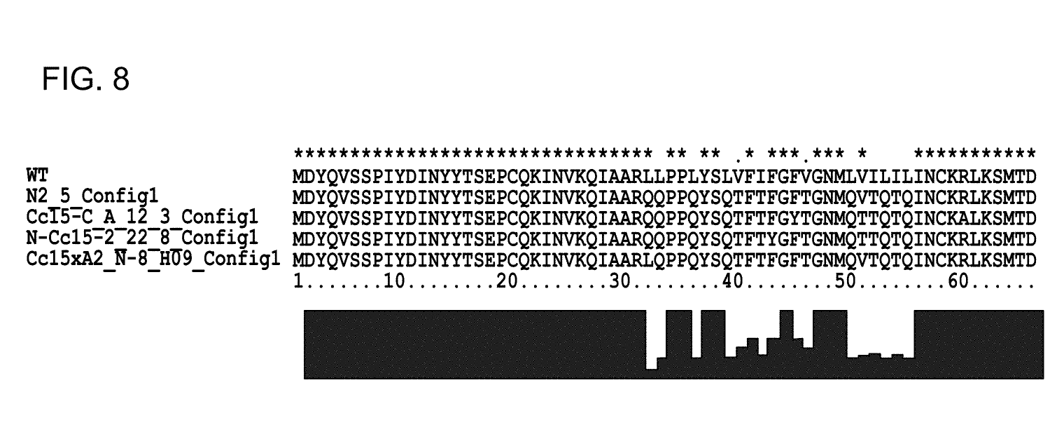

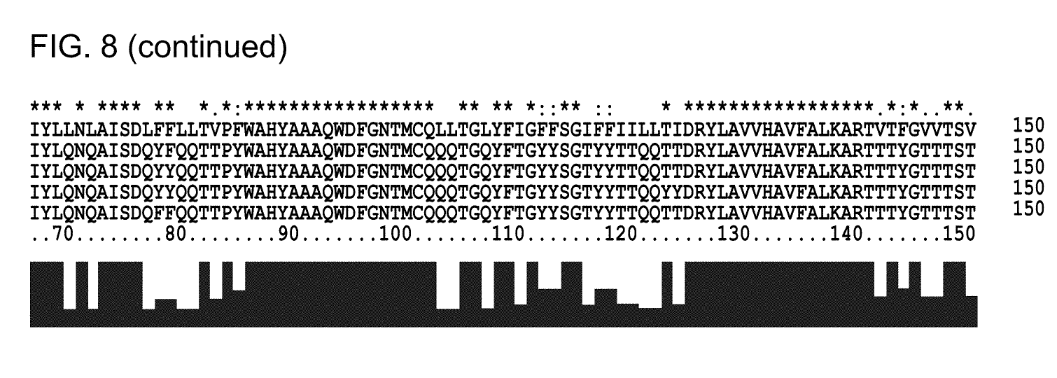

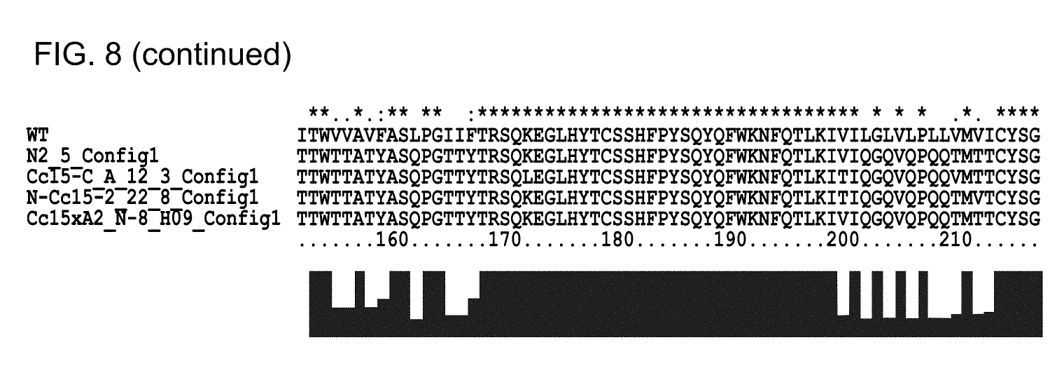

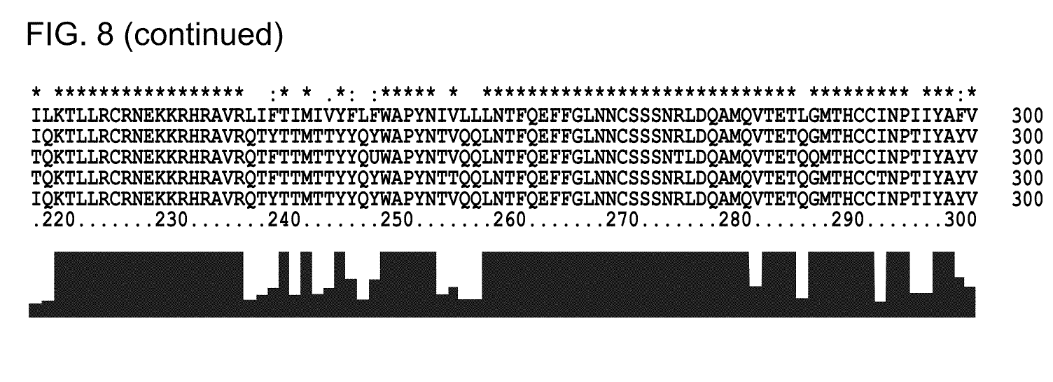

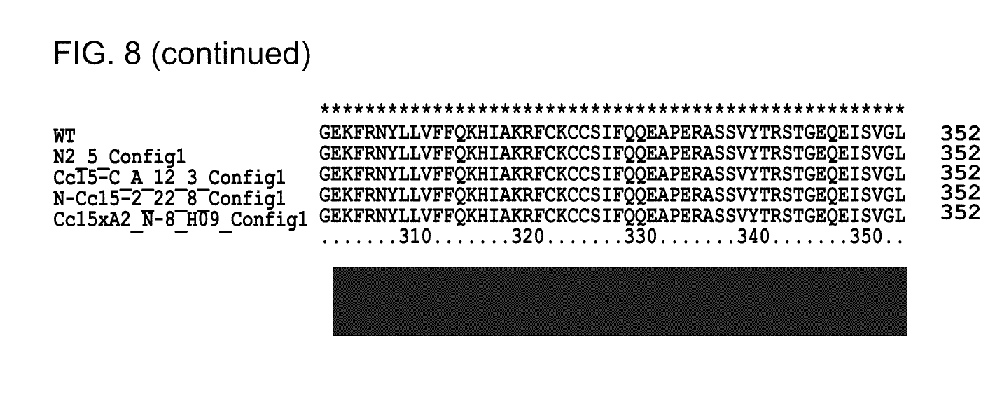

5: After identifying the TM data in the form (FIG. 5), the sequence of a GPCR is divided into total 15 fragments [7-transmembrane segments (FIG. 5A) and 8 non-transmembrane segments (FIG. 5B)] according to the TM region information in Step 5. Thus there should be 7TM and 8 NTM fragments for each typical GPCR.

It is understood that the system can execute one or more, such as all of the steps described above, using a computer interface for input by a user.

A first transmembrane region (typically, but not essentially, the transmembrane region which is most proximal to the N-terminal of the protein) is selected for variation. Hydrophobic amino acids (L, I, V, and F) are then substituted with the corresponding hydrophilic amino acid (Q, T or Y). It is understood that the amino acid is not actually substituted into the protein, in this context. Rather, the amino acid designation is substituted in the sequence for modeling. Thus, the term “sequence” is intended to include “sequence data.” Typically, most or all of the hydrophobic amino acids are selected for substitution. If less than all amino acids are selected, it may be desirable to select the internal hydrophobic amino acids leaving one or more N and/or C terminal amino acids of the transmembrane regions hydrophobic. Additionally or alternatively, it may be desirable to select to replace all of the leucines (L) in a transmembrane region. Additionally or alternatively, it may be desirable to select to replace all of the isoleucines (I) in a transmembrane region. Additionally or alternatively, it may be desirable to select to replace all of the valines (V) in a transmembrane region. Additionally or alternatively, it may be desirable to select to replace all of the phenylalanines (F) in a transmembrane region. Additionally or alternatively, it can be beneficial to retain one or more phenylalanines in the transmembrane region. Additionally or alternatively, it can be beneficial to retain one or more valines in the transmembrane region. Additionally or alternatively, it can be beneficial to retain one or more leucines in the transmembrane region. Additionally or alternatively, it can be beneficial to retain one or more isoleucines in the transmembrane region. Additionally or alternatively, it can be beneficial to retain one or more hydrophobic amino acids in the transmembrane region where the wild type sequence is characterized by three or more contiguous hydrophobic amino acids. The transmembrane region so designed (the transmembrane variant or “variant”) is then subjected to the transmembrane region prediction process, as discussed herein. The variant is then assessed a score for the sequence's propensity to form an alpha helix. The variant is also subjected to a water solubility prediction process, as discussed herein. The variant is assessed a score for the sequence's propensity to be water soluble. Of course, complete water solubility at all concentrations is not required for most commercial purposes. Water solubility is preferably determined to be that required for functionality at the predicted conditions of use (e.g., in a ligand binding assay).

Variants that predict loss of alpha helical structure and/or “water insolubility” (predicted at the expected conditions of use) are discarded. Variants that predict alpha helical structure and water solubility can be selected. One can select transmembrane variants that are highly water soluble, or are characterized by 0, 1, 2, or 3 hydrophobic amino acids, with a possible expectation that alpha helical structure can be compromised. Alternatively or additionally, one can select highly alpha-helical structures, characterized by 3, 4, 5 or 6 hydrophobic amino acids. These steps can be repeated for a second, third, fourth, fifth, sixth and/or seventh (or more) transmembrane region or domain.

One can select a combination of each domain where one, two, three or four domain variants possess high alpha-helical structure scores and one, two, three, four, five or six domain variants possess high water solubility scores. For example, one can choose a domain that is characterized by all hydrophobic amino acids being substituted by a hydrophilic amino acid, maximizing the water solubility score and a second domain variant selection that retains 3, 4, or 5 hydrophobic amino acids in a plurality of variant selections. Selected variants are then “shuffled,” as is known in the art, with the extracellular and intracellular domains to create an initial library of putative water-soluble protein variants.

All or a fraction of the putative water soluble protein variants of the initial library designed as described herein can be made and screened for water solubility and/or ligand binding, preferably in a high through-put screen. Amplification of the library, for example, can result in less than 100% of the putative water-soluble protein variants from being expressed. A reporter system can be used to screen ligand binding, as is well known in the art. Using the methods of the invention, one can rapidly identify a library of putative water soluble modified transmembrane variants that, when functionally combined with the extracellular and intracellular domains, will generate water soluble protein variants possessing the proper 3 dimensional structure of the wild type protein, to retain ligand binding function (including binding affinity), or other functions.

In order to be practical experimentally, we set out to make an initial library of about 2 million possible water-soluble GPCR, or CXCR4, variants. Of course, a library of more or less variants can be designed as well. Smaller libraries are preferred and can be optimized using routine experimentation based on analysis of the research results as described herein. Analysis of research results is likely to establish trends to optimize the number of domain variants to shuffle and the assumptions for selecting domain variants. Targeting transmembrane regions, we selected the amino acids for modifying based on the helical forming propensity also known as “the helix prediction score.” http://www.proteopedia.org/wiki/index.php/Main_Page. The varied fragments are randomly assembled to form about 2M (8⁷) variants of full-length GPCR genes. The predicted number of variants can be characterized by the formula Hⁿ, where n=the number of transmembrane regions modified and/or varied by the method (in this example, 7) and H=number of variants in each transmembrane region.

Once the initial library, or selection of the domain variants to be shuffled, is selected, nucleic acid molecules, or DNA or cDNA molecules, encoding the proteins in the initial library can be designed. The nucleic acid molecules are preferably designed to provide codon optimization and intron deletions for the expression systems selected to produce a library of coding sequences. For example, if the expression system is E. coli, codons optimized for E. coli expression can be selected. https://www.dna20.com/resources/genedesigner. In addition, a promoter region, such as a promoter suitable for expression in the expression system (e.g., E. coli) is selected and operatively connected to the coding sequences in the library of coding sequences.

The initial library of coding sequences, or a portion thereof, is then expressed to produce a library of putative water soluble GPCRs. The library is then subjected to a ligand binding assay. In the binding assay, the putative water soluble GPCRs are contacted with the ligand, preferably in an aqueous medium and ligand binding is detected.

The invention includes transmembrane domain variants, and nucleic acid molecules encoding same, obtained, or obtainable, from the methods described herein.

The invention contemplates water soluble GPCR variants (“sGPCRs”) characterized by a plurality of transmembrane domains independently characterized by at least 50%, preferably at least about 60%, more preferably at least about 70% or 80%, such as at least about 90%) of the hydrophobic amino acid residues (I/L, V and F) of a native transmembrane protein (e.g., GPCR) substituted by an T, Q or Y, respectively). The sGPCRs of the invention are characterized by water solubility and ligand binding. In particular, the sGPCR binds the same natural ligand as the corresponding native GPCR.

The invention further encompasses a method of treatment for a disorder or disease that is mediated by the activity of a membrane protein, comprising the use of a water-soluble polypeptide to treat said disorders and diseases, wherein said water-soluble polypeptide comprises a modified α-helical domain, and wherein said water-soluble polypeptide retains the ligand-binding activity of the native membrane protein. Examples of such disorders and diseases include, but are not limited to, cancer, small cell lung cancer, melanoma, breast cancer, Parkinson's disease, cardiovascular disease, hypertension, and asthma.

As described herein, the water-soluble peptides described herein can be used for the treatment of conditions or diseases mediated by the activity of a membrane protein. In certain aspects, the water-soluble peptides can act as “decoys” for the membrane receptor and bind to the ligand that otherwise activates the membrane receptor. As such, the water-soluble peptides described herein can be used to reduce the activity of a membrane protein. These water-soluble peptides can remain in the circulation and competitively bind to specific ligands, thereby reducing the activity of membrane bound receptors. For example, the GPCR CXCR4 is over-expressed in small cell lung cancer and facilitates metastasis of tumor cells. Binding of this ligand by a water-soluble peptide such as that described herein may significantly reduce metastasis.

The chemokine receptor, CXCR4, is known in viral research as a major coreceptor for the entry of T cell line-tropic HIV (Feng, et al. (1996) Science 272: 872-877; Davis, et al. (1997) J Exp Med 186: 1793-1798; Zaitseva, et al. (1997) Nat Med 3: 1369-1375; Sanchez, et al. (1997) J Biol Chem 272: 27529-27531). Stromal cell derived factor 1 (SDF-1) is a chemokine that interacts specifically with CXCR4. When SDF-1 binds to CXCR4, CXCR4 activates Gαi protein-mediated signaling (pertussis toxin-sensitive) (Chen, et al. (1998) Mol Pharmacol 53: 177-181), including downstream kinase pathways such as Ras/MAP Kinases and phosphatidylinositol 3-kinase (PI3K)/Akt in lymphocyte, megakaryocytes, and hematopoietic stem cells (Bleul, et al. (1996) Nature 382: 829-833; Deng, et al. (1997) Nature 388: 296-300; Kijowski, et al. (2001) Stem Cells 19: 453-466; Majka, et al. (2001) Folia. Histochem. Cytobiol. 39: 235-244; Sotsios, et al. (1999) J. Immunol. 163: 5954-5963; Vlahakis, et al. (2002) J. Immunol. 169: 5546-5554). In mice transplanted with human lymph nodes, SDF-1 induces CXCR4-positive cell migration into the transplanted lymph node (Blades, et al. (2002) J. Immunol. 168: 4308-4317).

Recently, studies have shown that CXCR4 interactions may regulate the migration of metastatic cells. Hypoxia, a reduction in partial oxygen pressure, is a microenvironmental change that occurs in most solid tumors and is a major inducer of tumor angiogenesis and therapeutic resistance. Hypoxia increases CXCR4 levels (Staller, et al. (2003) Nature 425: 307-311). Microarray analysis on a sub-population of cells from a bone metastatic model with elevated metastatic activity showed that one of the genes increased in the metastatic phenotype was CXCR4. Furthermore, overexpression CXCR4 in isolated cells significantly increased the metastatic activity (Kang, et al. (2003) Cancer Cell 3: 537-549). In samples collected from various breast cancer patients, Muller et al. (Muller, et al. (2001) Nature 410: 50-56) found that CXCR4 expression level is higher in primary tumors relative to normal mammary gland or epithelial cells. Moreover, CXCR4 antibody treatment has been shown to inhibit metastasis to regional lymph nodes when compared to control isotypes that all metastasized to lymph nodes and lungs (Muller, et al. (2001)). As such a decoy therapy model is suitable for treating CXCR4 mediated diseases and disorders.

In another embodiment of the invention relates to the treatment of a disease or disorder involving CXCR4-dependent chemotaxis, wherein the disease is associated with aberrant leukocyte recruitment or activation. The disease is selected from the group consisting of arthritis, psoriasis, multiple sclerosis, ulcerative colitis, Crohn's disease, allergy, asthma, AIDS associated encephalitis, AIDS related maculopapular skin eruption, AIDS related interstitial pneumonia, AIDS related enteropathy, AIDS related periportal hepatic inflammation and AIDS related glomerulo nephritis.

In another aspect, the invention relates to the treatment of a disease or disorder selected from arthritis, lymphoma, non-small lung cancer, lung cancer, breast cancer, prostate cancer, multiple sclerosis, central nervous system developmental disease, dementia, Parkinson's disease, Alzheimer's disease, tumor, fibroma, astrocytoma, myeloma, glioblastoma, an inflammatory disease, an organ transplantation rejection, AIDS, HIV-infection or angiogenesis.

The invention also encompasses a pharmaceutical composition comprising said water-soluble polypeptide and a pharmaceutically acceptable carrier or diluent.

The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the pharmacologic agent or composition. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Pharmaceutical compositions can also include large, slowly metabolized macromolecules such as proteins, polysaccharides such as chitosan, polylactic acids, polyglycolic acids and copolymers (such as latex functionalized SEPHAROSE™, agarose, cellulose, and the like), polymeric amino acids, amino acid copolymers, and lipid aggregates (such as oil droplets or liposomes).

The compositions can be administered parenterally such as, for example, by intravenous, intramuscular, intrathecal or subcutaneous injection. Parenteral administration can be accomplished by incorporating a composition into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Injectable formulations can be prepared either as liquid solutions or suspensions; solid forms suitable for solution in, or suspension in, liquid vehicles prior to injection can also be prepared. The preparation also can also be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The compositions and pharmacologic agents described herein can be administered in the form of a depot injection or implant preparation which can be formulated in such a manner as to permit a sustained or pulsatile release of the active ingredient.

Transdermal administration includes percutaneous absorption of the composition through the skin. Transdermal formulations include patches, ointments, creams, gels, salves and the like. Transdermal delivery can be achieved using a skin patch or using transferosomes. [Paul et al., Eur. J. Immunol. 25: 3521-24, 1995; Cevc et al., Biochem. Biophys. Acta 1368: 201-15, 1998].

“Treating” or “treatment” includes preventing or delaying the onset of the symptoms, complications, or biochemical indicia of a disease, alleviating or ameliorating the symptoms or arresting or inhibiting further development of the disease, condition, or disorder. A “patient” is a human subject in need of treatment.

An “effective amount” refers to that amount of the therapeutic agent that is sufficient to ameliorate of one or more symptoms of a disorder and/or prevent advancement of a disorder, cause regression of the disorder and/or to achieve a desired effect.

### Computer System

Various aspects and functions described herein may be implemented as specialized hardware or software components executing in one or more computer systems. There are many examples of computer systems that are currently in use. These examples include, among others, network appliances, personal computers, workstations, mainframes, networked clients, servers, media servers, application servers, database servers, and web servers. Other examples of computer systems may include mobile computing devices, such as cellular phones and personal digital assistants, and network equipment, such as load balancers, routers, and switches. Further, aspects may be located on a single computer system or may be distributed among a plurality of computer systems connected to one or more communications networks.

For example, various aspects, functions, and processes may be distributed among one or more computer systems configured to provide a service to one or more client computers, or to perform an overall task as part of a distributed system. Additionally, aspects may be performed on a client-server or multi-tier system that includes components distributed among one or more server systems that perform various functions. Consequently, embodiments are not limited to executing on any particular system or group of systems. Further, aspects, functions, and processes may be implemented in software, hardware or firmware, or any combination thereof. Thus, aspects, functions, and processes may be implemented within methods, acts, systems, system elements and components using a variety of hardware and software configurations, and examples are not limited to any particular distributed architecture, network, or communication protocol.

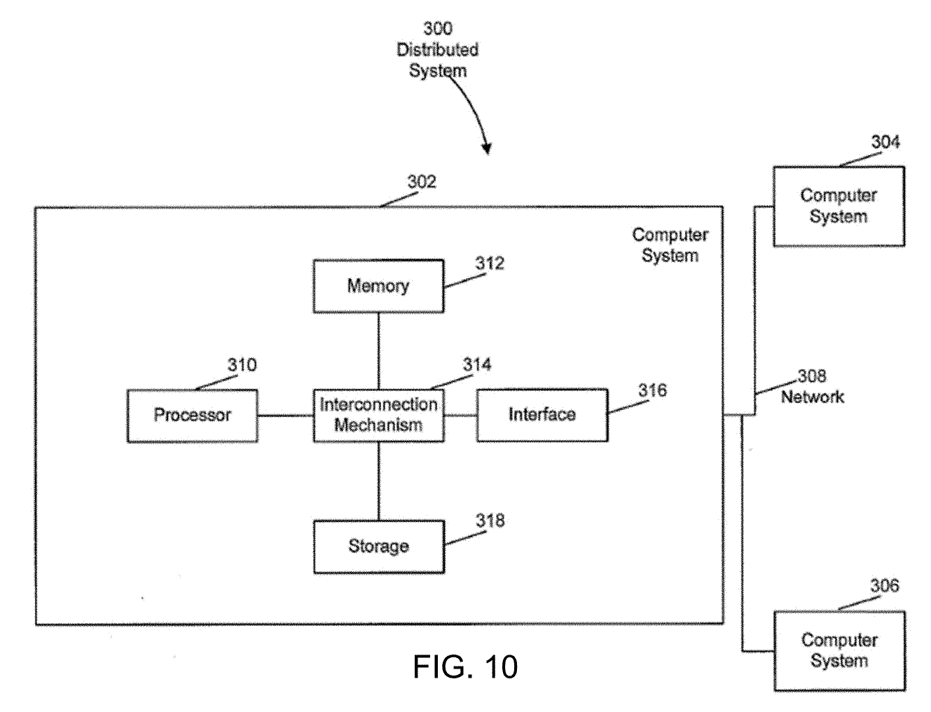

Referring to FIG. 10, there is illustrated a block diagram of a distributed computer system 300, in which various aspects and functions are practiced. As shown, the distributed computer system 300 includes one or more computer systems that exchange information. More specifically, the distributed computer system 300 includes computer systems 302, 304, and 306. As shown, the computer systems 302, 304, and 306 are interconnected by, and may exchange data through, a communication network 308. The network 308 may include any communication network through which computer systems may exchange data. To exchange data using the network 308, the computer systems 302, 304, and 306 and the network 308 may use various methods, protocols and standards. Examples of these protocols and standards include NAS, Web, storage and other data movement protocols suitable for use in a big data environment. To ensure data transfer is secure, the computer systems 302, 304, and 306 may transmit data via the network 308 using a variety of security measures including, for example, SSL or VPN technologies. While the distributed computer system 300 illustrates three networked computer systems, the distributed computer system 300 is not so limited and may include any number of computer systems and computing devices, networked using any medium and communication protocol.

As illustrated in FIG. 10, the computer system 302 includes a processor 310, a memory 312, an interconnection element 314, an interface 316 and data storage element 318. To implement at least some of the aspects, functions, and processes disclosed herein, the processor 310 performs a series of instructions that result in manipulated data. The processor 310 may be any type of processor, multiprocessor or controller. Example processors may include a commercially available processor such as an Intel Xeon, Itanium, Core, Celeron, or Pentium processor; an AMD Opteron processor; an Apple A4 or A5 processor; a Sun UltraSPARC processor; an IBM Power5+ processor; an IBM mainframe chip; or a quantum computer. The processor 310 is connected to other system components, including one or more memory devices 312, by the interconnection element 314.

The memory 312 stores programs (e.g., sequences of instructions coded to be executable by the processor 310) and data during operation of the computer system 302. Thus, the memory 312 may be a relatively high performance, volatile, random access memory such as a dynamic random access memory (“DRAM”) or static memory (“SRAM”). However, the memory 312 may include any device for storing data, such as a disk drive or other nonvolatile storage device. Various examples may organize the memory 312 into particularized and, in some cases, unique structures to perform the functions disclosed herein. These data structures may be sized and organized to store values for particular data and types of data.

Components of the computer system 302 are coupled by an interconnection element such as the interconnection element 314. The interconnection element 314 may include any communication coupling between system components such as one or more physical busses in conformance with specialized or standard computing bus technologies such as IDE, SCSI, PCI and InfiniBand. The interconnection element 314 enables communications, including instructions and data, to be exchanged between system components of the computer system 302.

The computer system 302 also includes one or more interface devices 316 such as input devices, output devices and combination input/output devices. Interface devices may receive input or provide output. More particularly, output devices may render information for external presentation. Input devices may accept information from external sources. Examples of interface devices include keyboards, mouse devices, trackballs, microphones, touch screens, printing devices, display screens, speakers, network interface cards, etc. Interface devices allow the computer system 302 to exchange information and to communicate with external entities, such as users and other systems.

The data storage element 318 includes a computer readable and writeable nonvolatile, or non-transitory, data storage medium in which instructions are stored that define a program or other object that is executed by the processor 310. The data storage element 318 also may include information that is recorded, on or in, the medium, and that is processed by the processor 310 during execution of the program. More specifically, the information may be stored in one or more data structures specifically configured to conserve storage space or increase data exchange performance. The instructions may be persistently stored as encoded signals, and the instructions may cause the processor 310 to perform any of the functions described herein. The medium may, for example, be optical disk, magnetic disk or flash memory, among others. In operation, the processor 310 or some other controller causes data to be read from the nonvolatile recording medium into another memory, such as the memory 312, that allows for faster access to the information by the processor 310 than does the storage medium included in the data storage element 318. The memory may be located in the data storage element 318 or in the memory 312, however, the processor 310 manipulates the data within the memory, and then copies the data to the storage medium associated with the data storage element 318 after processing is completed. A variety of components may manage data movement between the storage medium and other memory elements and examples are not limited to particular data management components. Further, examples are not limited to a particular memory system or data storage system.

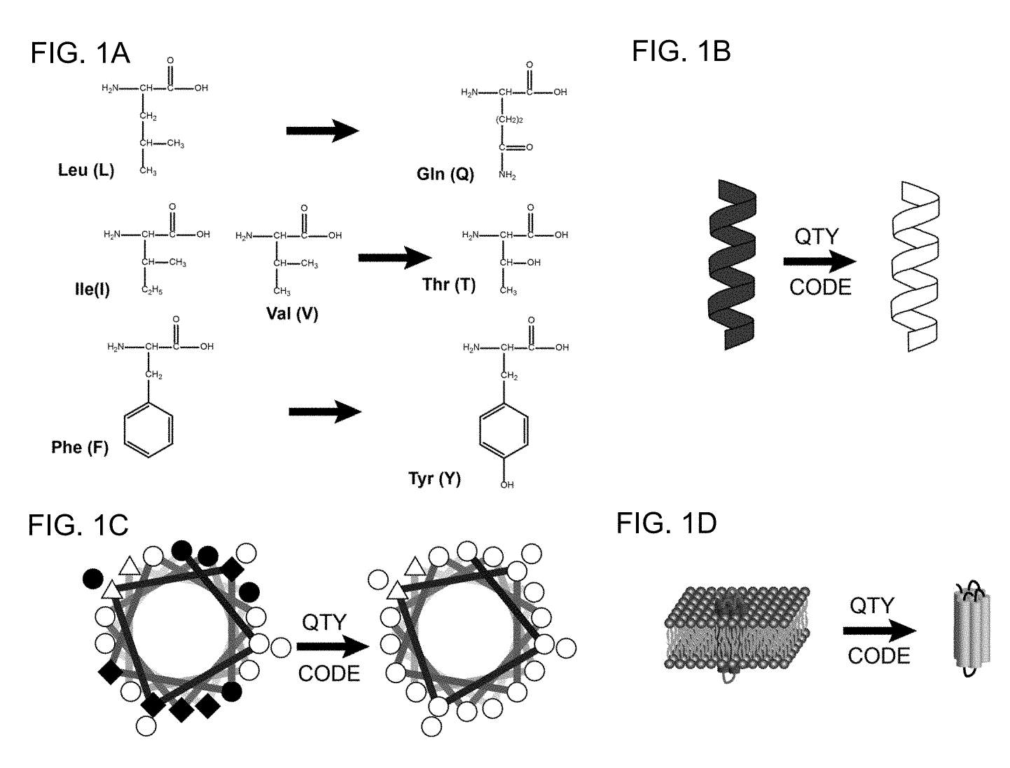

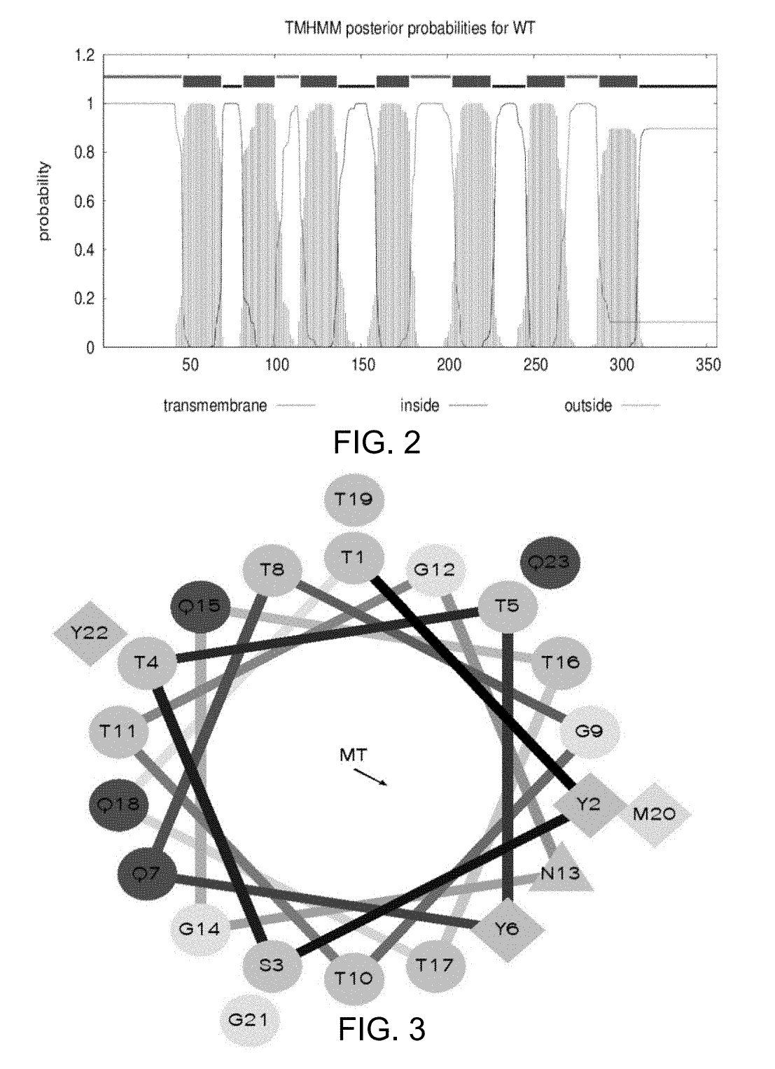

Although the computer system 302 is shown by way of example as one type of computer system upon which various aspects and functions may be practiced, aspects and functions are not limited to being implemented on the computer system 302 as shown in FIG. 1. Various aspects and functions may be practiced on one or more computers having a different architectures or components than that shown in FIG. 1. For instance, the computer system 302 may include specially programmed, special-purpose hardware, such as an application-specific integrated circuit (“ASIC”) tailored to perform a particular operation disclosed herein. While another example may perform the same function using a grid of several general-purpose computing devices running MAC OS System X with Motorola PowerPC processors and several specialized computing devices running proprietary hardware and operating systems.

The computer system 302 may be a computer system including an operating system that manages at least a portion of the hardware elements included in the computer system 302. In some examples, a processor or controller, such as the processor 310, executes an operating system. Examples of a particular operating system that may be executed include a Windows-based operating system, such as, Windows NT, Windows 2000 (Windows ME), Windows XP, Windows Vista or Windows 7 operating systems, available from the Microsoft Corporation, a MAC OS System X operating system or an iOS operating system available from Apple Computer, one of many Linux-based operating system distributions, for example, the Enterprise Linux operating system available from Red Hat Inc., a Solaris operating system available from Oracle Corporation, or a UNIX operating systems available from various sources. Many other operating systems may be used, and examples are not limited to any particular operating system.

The processor 310 and operating system together define a computer platform for which application programs in high-level programming languages are written. These component applications may be executable, intermediate, bytecode or interpreted code which communicates over a communication network, for example, the Internet, using a communication protocol, for example, TCP/IP. Similarly, aspects may be implemented using an object-oriented programming language, such as .Net, SmallTalk, Java, C++, Ada, C# (C-Sharp), Python, or JavaScript. Other object-oriented programming languages may also be used. Alternatively, functional, scripting, or logical programming languages may be used.

Additionally, various aspects and functions may be implemented in a non-programmed environment. For example, documents created in HTML, XML or other formats, when viewed in a window of a browser program, can render aspects of a graphical-user interface or perform other functions. Further, various examples may be implemented as programmed or non-programmed elements, or any combination thereof. For example, a web page may be implemented using HTML while a data object called from within the web page may be written in C++. Thus, the examples are not limited to a specific programming language and any suitable programming language could be used. Accordingly, the functional components disclosed herein may include a wide variety of elements (e.g., specialized hardware, executable code, data structures or objects) that are configured to perform the functions described herein.

In some examples, the components disclosed herein may read parameters that affect the functions performed by the components. These parameters may be physically stored in any form of suitable memory including volatile memory (such as RAM) or nonvolatile memory (such as a magnetic hard drive). In addition, the parameters may be logically stored in a propriety data structure (such as a database or file defined by a user space application) or in a commonly shared data structure (such as an application registry that is defined by an operating system). In addition, some examples provide for both system and user interfaces that allow external entities to modify the parameters and thereby configure the behavior of the components.

The invention will be better understood in connection with the following example, which is intended as an illustration only and not limiting of the scope of the invention. Various changes and modifications to the disclosed embodiments will be apparent to those skilled in the art and such changes and may be made without departing from the spirit of the invention and the scope of the appended claims.

### Examples

### Example 1: CXC Chemokine Receptor Type 4 Isoform a (CXCR4)

CXCR4 is a chemokine receptor 356 amino acids in length. It has a pI of about 8.61 and a Molecular Weight of 40221.19 Da. The sequence for CXCR4, as published in the literature, is:

(SEQ ID NO. 1)

MSIPLPLLQIYTSDNYTEEMGSGDYDSMKEPCFREENANFNKIFLPTIYS
IIFLTGIVGNGLVILVMGYQKKLRSMTDKYRLHLSVADLLFVITLPFWAV
DAVANWYFGNFLCKAVHVIYTVNLYSSVLILAFISLDRYLAIVHATNSQR
PRKLLAEKVVYVGVWIPALLLTIPDFIFANVSEADDRYICDRFYPNDLWV
VVFQFQHIMVGLILPGIVILSCYCIIISKLSHSKGHQKRKALKTTVILIL
AFFACWLPYYIGISIDSFILLEIIKQGCEFENTVHKWISITEALAFFHCC
LNPILYAFLGAKFKTSAQHALTSVSRGSSLKILSKGKRGGHSSVSTESES
SSFHSS.

Subjecting the sequence to TMHMM results in the identification of the transmembrane domains as depicted in FIG. 3.

Replacing all or substantially all of the hydrophobic amino acids, L, I V, and F, with Q, T and Y (respectively) results in the following sequence:

| (SEQ ID NO: 2) | |
|---|---|
| 1 | MSIPLPLLQIYTSDNYTEEMGSGDYDSMKEPCFREENANFNKIFLPTTYSTTYQTGTTGN |
| 61 | GQTTQTMGYQKKLRSMTDKYRQHQSTADQQYTTTQPYWATDAVANWYFGNFLCKATHTTY |
| 121 | TTNQYSSTQTQAYTSQDRYLAIVHATNSQRPRKLLAEKTTYTGTWTPAQQQTTPDYTYAN |
| 181 | VSEADDRYICDRFYPNDLWVVVYQYQHTMTGQTQPGTTTQSCYCTIISKLSHSKGHQKRK |
| 241 | ALKTTTTQTQAYYACWQPYYTGTSTDSYILLEIIKQGCEFENTVHKWTSTTEAQAYYHCC |
| 301 | QNPTQYAYQGAKFKTSAQHALTSVSRGSSLKILSKGKRGGHSSVSTESESSSFHSS. |

The predicted pI of the protein is 8.54 and the Molecular Weight is 40551.64 Da. Each of the predicted transmembrane regions has been underlined and exemplified a fully modified domain of the invention. Thus, for example, the invention includes a transmembrane domain comprising Amino Acids 47-70 of SEQ ID NO: 2 (TM1), and proteins comprising the same. As an example, FIG. 3 represents the alpha-helical prediction of the TM1 sequence. Preferably the protein comprising TM1 herein includes one or more (e.g., all) of the extracellular and intracellular loop sequences of SEQ ID NO: 2 (the sequences which have not been underlined). In addition or alternatively, the protein comprising the TM1 herein includes one or more additional transmembrane regions (the underlined sequences) in SEQ ID NO: 2 or homologous sequences retaining one, two, three or, possibly four or more of the native L, I V and F amino acids, as set forth in SEQ ID NO: 1.

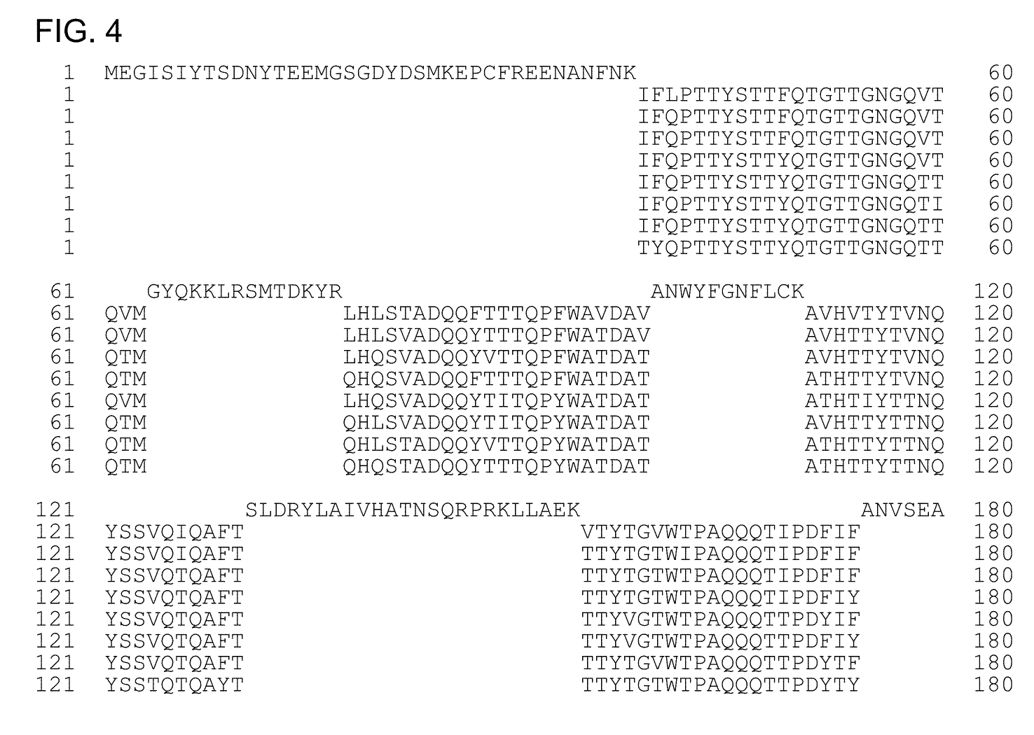

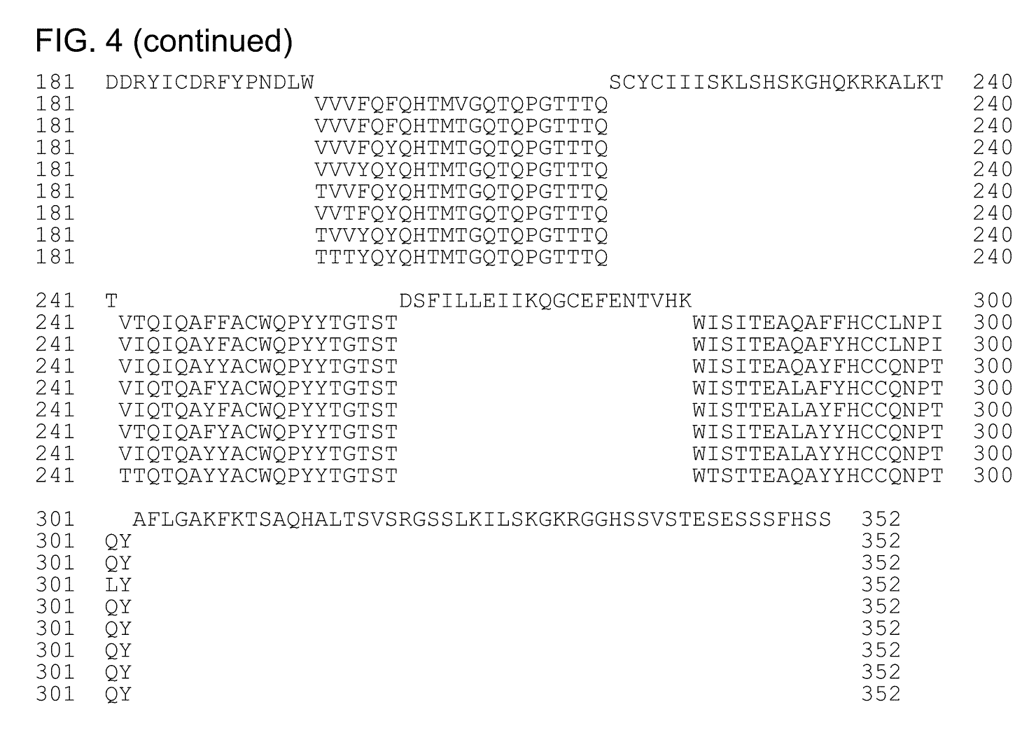

The native protein sequence for CXCR4 (differing in the N-terminal amino acids) was subjected to the method a second time. The program output divided the native sequence into the extracellular and intracellular regions and selected 8 transmembrane domain variants for each transmembrane domain. The results are illustrated in FIG. 4 and in the following table:

| | |
|---|---|
| MEGISIYTSDNYTEEMGSGDYDSMKEPCF | (SEQ ID NO. 3; EC1) |
| REENANFNK | |
| TM 1 Variants: | |
| IFLPTTYSTTFQTGTTGNGQVTQVM | (SEQ ID NO. 4) |
| IFQPTTYSTTFQTGTTGNGQVTQVM | (SEQ ID NO. 5) |
| IFQPTTYSTTFQTGTTGNGQVTQTM | (SEQ ID NO. 6) |
| IFQPTTYSTTYQTGTTGNGQVTQTM | (SEQ ID NO. 7) |
| IFQPTTYSTTYQTGTTGNGQTTQVM | (SEQ ID NO. 8) |
| IFQPTTYSTTYQTGTTGNGQTIQTM | (SEQ ID NO. 9) |
| IFQPTTYSTTYQTGTTGNGQTTQTM | (SEQ ID NO. 10) |
| TYQPTTYSTTYQTGTTGNGQTTQTM | (SEQ ID NO. 11) |
| GYQKKLRSMTDKYR | (SEQ ID NO. 12; IC1) |
| TM 2 Variants: | |
| LHLSTADQQFTTTQPFWAVDAV | (SEQ ID NO. 13) |
| LHLSVADQQYTTTQPFWATDAV | (SEQ ID NO. 14) |
| LHQSVADQQYVTTQPFWATDAT | (SEQ ID NO. 15) |
| QHQSVADQQFTTTQPFWATDAT | (SEQ ID NO. 16) |
| LHQSVADQQYTITQPYWATDAT | (SEQ ID NO. 17) |
| QHLSVADQQYTITQPYWATDAT | (SEQ ID NO. 18) |
| QHLSTADQQYVTTQPYWATDAT | (SEQ ID NO. 19) |
| QHQSTADQQYTTTQPYWATDAT | (SEQ ID NO. 20) |
| ANWYFGNFLCK | (SEQ ID NO. 21; EC2) |
| TM 3 Variants: | |
| AVHVTYTVNQYSSVQIQAFT | (SEQ ID NO. 22) |
| AVHTTYTVNQYSSVQIQAFT | (SEQ ID NO. 23) |
| AVHTTYTVNQYSSVQTQAFT | (SEQ ID NO. 24) |
| ATHTTYTVNQYSSVQTQAFT | (SEQ ID NO. 25) |
| ATHTIYTTNQYSSVQTQAFT | (SEQ ID NO. 26) |
| AVHTTYTTNQYSSVQTQAFT | (SEQ ID NO. 27) |
| ATHTTYTTNQYSSVQTQAFT | (SEQ ID NO. 28) |
| ATHTTYTTNQYSSTQTQAYT | (SEQ ID NO. 29) |
| SLDRYLAIVHATNSQRPRKLLAEK | (SEQ ID NO. 30; IC2) |
| TM 4 Variants: | |
| VTYTGVWTPAQQQTIPDFIF | (SEQ ID NO. 31) |
| TTYTGTWIPAQQQTIPDFIF | (SEQ ID NO. 32) |
| TTYTGTWTPAQQQTIPDFIF | (SEQ ID NO. 33) |
| TTYTGTWTPAQQQTIPDFIY | (SEQ ID NO. 34) |
| TTYVGTWTPAQQQTTPDYIF | (SEQ ID NO. 35) |
| TTYVGTWTPAQQQTTPDFIY | (SEQ ID NO. 36) |
| TTYTGVWTPAQQQTTPDYTF | (SEQ ID NO. 37) |
| TTYTGTWTPAQQQTTPDYTY | (SEQ ID NO. 38) |
| ANVSEADDRYICDRFYPNDLW | (SEQ ID NO. 39; EC3) |
| TM 5 Variants: | |
| VVVFQFQHTMVGQTQPGTTTQ | (SEQ ID NO. 40) |
| VVVFQFQHTMTGQTQPGTTTQ | (SEQ ID NO. 41) |
| VVVFQYQHTMTGQTQPGTTTQ | (SEQ ID NO. 42) |
| VVVYQYQHTMTGQTQPGTTTQ | (SEQ ID NO. 43) |
| TVVFQYQHTMTGQTQPGTTTQ | (SEQ ID NO. 44) |
| VVTFQYQHTMTGQTQPGTTTQ | (SEQ ID NO. 45) |
| TVVYQYQHTMTGQTQPGTTTQ | (SEQ ID NO. 46) |
| TTTYQYQHTMTGQTQPGTTTQ | (SEQ ID NO. 47) |
| SCYCIIISKLSHSKGHQKRKALKTT | (SEQ ID NO. 48; IC3) |
| TM 6 Variants: | |
| VTQIQAFFACWQPYYTGTST | (SEQ ID NO. 49) |
| VIQIQAYFACWQPYYTGTST | (SEQ ID NO. 50) |
| VIQIQAYYACWQPYYTGTST | (SEQ ID NO. 51) |
| VIQTQAFYACWQPYYTGTST | (SEQ ID NO. 52) |
| VIQTQAYFACWQPYYTGTST | (SEQ ID NO. 53) |
| VTQIQAFYACWQPYYTGTST | (SEQ ID NO. 54) |
| VIQTQAYYACWQPYYTGTST | (SEQ ID NO. 55) |
| TTQTQAYYACWQPYYTGTST | (SEQ ID NO. 56) |
| DSFILLEIIKQGCEFENTVHK | (SEQ ID NO. 57; EC4) |
| TM 7 Variants | |
| WISITEAQAFFHCCLNPIQY | (SEQ ID NO. 58) |
| WISITEAQAFYHCCLNPIQY | (SEQ ID NO. 59) |
| WISITEAQAYFHCCQNPTLY | (SEQ ID NO. 60) |
| WISTTEALAFYHCCQNPTQY | (SEQ ID NO. 61) |
| WISTTEALAYFHCCQNPTQY | (SEQ ID NO. 62) |
| WISITEALAYYHCCQNPTQY | (SEQ ID NO. 63) |
| WISTTEALAYYHCCQNPTQY | (SEQ ID NO. 64) |
| WTSTTEAQAYYHCCQNPTQY | |
| AFLGAKFKTSAQHALTSVSRGSSLKILS | (SEQ ID NO. 65; IC4) |
| KGKRGGHSSVSTESESSSFHSS. | |

It is believed that it is clear from the above, that the sequences (SEQ ID NOs: 3, 12, 21, 30, 39, 48, 57 and 65) before, between and after each list of transmembrane domain variants are the N′, intermediary and C′ extracellular and intracellular regions, respectively.

The sequences above were then used to generate coding sequences, as is known in the art, suitable for expression in the expression system, in this case yeast. The coding sequences were then shuffled and expressed to produce a library comprising a plurality of proteins each having SEQ ID NOs: 3, 12, 21, 30, 39, 48, 57 and 65 with one transmembrane domain variant from each variant list in between the respective intracellular and extracellular domain.

The library so produced was then assayed for CXCR4 cognate ligand, SDF1a (or CCL12) on a plasmid expressed in yeast binding inside living yeast cells. Ligand binding was detected by gene activation from the yeast 2-hybrid system and samples were then sequenced. Nineteen CXCR4 variants were sequenced. The results are shown in FIG. 5.

### Example 2: CXC Chemokine Receptor Type 3 Isoform b (CX3CR1)

CX3CR1 is a chemokine receptor 355 amino acids in length. It has a pI of about 6.74 and a Molecular Weight of 40396.4 Da. The subjecting of the sequence to TMHMM results in the identification of the transmembrane domains. Replacing all or substantially all of the hydrophobic amino acids, L, I V, and F, with Q, T and Y (respectively) within the transmembrane domains results in the following sequence (lower line), aligned with the wild type (top line):

MDQFPESVTENFEYDDLAEACYIGDIVVFGTVFLSIFYSVIFAIGLVGNLLVVFALTNSK
|||||||||||||||||||||||||||||||||*|**||***|*|**||*****|*||||
MDQFPESVTENFEYDDLAEACYIGDIVVFGTVFQSTYYSTTYATGQTGNQQTTYAQTNSK
KPKSVTDIYLLNLALSDLLFVATLPFWTHYLINEKGLHNAMCKFTTAFFFIGFFGSIFFI
|||||||*|**|*|*||****||*|*||||*||||||||||||||||****|**||****
KPKSVTDTYQQNQAQSDQQYTATQPYWTHYQINEKGLHNAMCKFTTAYYYTGYYGSTYYT
TVISIDRYLAIVLAANSMNNRTVQHGVTISLGVWAAAILVAAPQFMFTKQKENECLGDYP
|**|*|||||||||||||||||||||*|*|*|*||||***||||*|*|||||||||||||
TTTSTDRYLAIVLAANSMNNRTVQHGTTTSQGTWAAATQTAAPQYMYTKQKENECLGDYP
EVLQEIWPVLRNVETNFLGFLLPLLIMSYCYFRIIQTLFSCKNHKKAKAIKLILLVVIVF
||||||||||||||||**|***|***|||||*|**||**||||||||||||*********
EVLQEIWPVLRNVETNYQGYQQPQQTMSYCYYRTTQTQYSCKNHKKAKAIKQTQQTTTTY
FLFWTPYNVMIFLETLKLYDFFPSCDMRKDLRLALSVTETVAFSHCCLNPLIYAFAGEKF
***|||||*|***|||||||||||||||||||||*|*|||*|*||||*||**||*|||||
YQYWTPYNTMTYQETLKLYDFFPSCDMRKDLRLAQSTTETTAYSHCCQNPQTYAYAGEKF
RRYLYHLYGKCLAVLCGRSVHVDFSSSESQRSRHGSVLSSNFTYHTSDGDALLLL (SEQ ID NO. 66)
|||||||||||||||||||||||||||||||||||||||||||||||||||||||
RRYLYHLYGKCLAVLCGRSVHVDFSSSESQRSRHGSVLSSNFTYHTSDGDALLLL. (SEQ ID NO. 67)

The predicted pI of the protein variant is 6.74 and the Molecular Weight is 41027.17 Da. Each of the predicted transmembrane regions has been underlined and exemplified a fully modified domain of the invention. Thus, for example, the invention includes a transmembrane domain comprising the underlined Amino Acids of SEQ ID NO: 67. Preferably the protein comprising TM1 herein includes one or more (e.g., all) of the extracellular and intracellular loop sequences of SEQ ID NO: 66 (the sequences which have not been underlined). In addition or alternatively, the protein comprising the TM1 herein includes one or more additional transmembrane regions (the underlined sequences) in SEQ ID NO: 67 or homologous sequences retaining one, two, three or, possibly four or more of the native V, L, I and F amino acids, as set forth in SEQ ID NO: 66.

The native protein sequence for CX3CR1 was subjected to the method a second time. The program output divided the native sequence into the extracellular and intracellular regions and selected 8 transmembrane domain variants for each transmembrane domain. The results are illustrated in the following table:

| | |
|---|---|
| MDQFPESVTENFEYDDLAEACYIGDIVVFGT | (SEQ ID NO. 68) |
| TM 1 Variants: | |
| TYQSTYYSTTFATGQVGNQQVVFALTNS | (SEQ ID NO. 69) |
| TYQSTYYSTTYATGQVGNQQVVFALTNS | (SEQ ID NO. 70) |
| TYQSTYYSTTYATGQVGNQQVVFAQTNS | (SEQ ID NO. 71) |
| TYQSTYYSTTYATGQTGNLQVTFAQTNS | (SEQ ID NO. 72) |
| TYQSTYYSTTYATGQTGNQLVTFAQTNS | (SEQ ID NO. 73) |
| TYQSTYYSTTYATGQTGNQQVVFAQTNS | (SEQ ID NO. 74) |
| TYQSTYYSTTYATGQTGNLQVTYAQTNS | (SEQ ID NO. 75) |
| TYQSTYYSTTYATGQTGNQQTTYAQTNS | (SEQ ID NO. 76) |
| KKPKSVTDIY | (SEQ ID NO. 77) |
| TM 2 Variants | |
| LLNQAQSDQLFVATQPFWTHY | (SEQ ID NO. 78) |
| LLNQAQSDQQFVATQPFWTHY | (SEQ ID NO. 79) |
| QQNLAQSDQQFVATQPFWTHY | (SEQ ID NO. 80) |
| LQNLAQSDQQYTATQPFWTHY | (SEQ ID NO. 81) |
| QLNLAQSDQQYTATQPFWTHY | (SEQ ID NO. 82) |
| LLNQAQSDQQFTATQPYWTHY | (SEQ ID NO. 83) |
| QQNLAQSDQQFTATQPYWTHY | (SEQ ID NO. 84) |
| QQNQAQSDQQYTATQPYWTHY | (SEQ ID NO. 85) |
| LINEKGLHNAMCK | (SEQ ID NO. 86) |
| TM3 Variant | |
| YTTAYYYTGYYGSTYYTTTTST | (SEQ ID NO. 87) |
| DRYLAIVLAANSMNNRT | (SEQ ID NO. 88) |
| TM4 Variants: | |
| VQHGTTTSQGTWAAATQVAAPQFMF | (SEQ ID NO. 89) |
| VQHGVTTSQGTWAAATQTAAPQFMF | (SEQ ID NO. 90) |
| VQHGTTTSQGVWAAATQTAAPQFMY | (SEQ ID NO. 91) |
| VQHGTTTSQGTWAAAIQTAAPQFMY | (SEQ ID NO. 92) |
| VQHGTTTSQGTWAAATQTAAPQFMF | (SEQ ID NO. 93) |
| VQHGTTISQGTWAAATQTAAPQYMF | (SEQ ID NO. 94) |
| VQHGTTTSQGTWAAATQTAAPQFMY | (SEQ ID NO. 95) |
| TQHGTTTSQGTWAAATQTAAPQYMY | (SEQ ID NO. 96) |
| TKQKENECLGDYPEVLQEIWPVLRNVET | (SEQ ID NO. 97) |
| TM5 Variants: | |
| NFLGFQQPQQIMSYCYFRIT | (SEQ ID NO. 98) |
| NFQGFLQPQQTMSYCYFRIT | (SEQ ID NO. 99) |
| NFQGFLQPQQTMSYCYFRTT | (SEQ ID NO. 100) |
| NFQGFQQPQQTMSYCYYRIT | (SEQ ID NO. 101) |
| NFQGFLQPQQTMSYCYYRTT | (SEQ ID NO. 102) |
| NFQGYLQPQQTMSYCYFRTT | (SEQ ID NO. 103) |
| NYQGFQQPQQTMSYCYFRTT | (SEQ ID NO. 104) |
| NYQGYQQPQQTMSYCYYRTT | (SEQ ID NO. 105) |
| QTLFSCKNHKKAKAIK | (SEQ ID NO. 106) |
| TM6 Variants: | |
| LIQQTTTTFYQFWTPYNTMTFQETL | (SEQ ID NO. 107) |
| LIQQTTTTFYQYWTPYNVMTFQETQ | (SEQ ID NO. 108) |
| LIQQTTTTYYQFWTPYNTMTFQETQ | (SEQ ID NO. 109) |
| QIQQTTTTFYQYWTPYNTMTFQETQ | (SEQ ID NO. 110) |
| LTQQTTTTYYQFWTPYNTMTFQETQ | (SEQ ID NO. 111) |
| QIQQTTTTFFQYWTPYNTMTYQETQ | (SEQ ID NO. 112) |
| QIQQTTTTFYQYWTPYNTMTYQETQ | (SEQ ID NO. 113) |
| QTQQTTTTYYQYWTPYNTMTYQETQ | (SEQ ID NO. 114) |
| KLYDFFPSCDMRKDLRL | (SEQ ID NO. 115) |
| TM7 Variants: | |
| ALSVTETVAFSHCCQNPQIYAFAG | (SEQ ID NO. 116) |
| AQSVTETTAFSHCCQNPLIYAFAG | (SEQ ID NO. 117) |
| ALSVTETVAFSHCCQNPQTYAYAG | (SEQ ID NO. 118) |
| AQSVTETTAFSHCCQNPQIYAYAG | (SEQ ID NO. 119) |
| ALSVTETTAFSHCCQNPQTYAYAG | (SEQ ID NO. 120) |
| ALSTTETTAYSHCCQNPQIYAFAG | (SEQ ID NO. 121) |
| ALSVTETTAYSHCCQNPQTYAYAG | (SEQ ID NO. 122) |
| AQSTTETTAYSHCCQNPQTYAYAG | (SEQ ID NO. 123) |
| EKFRRYLYHLYGKCLAVLCGRSVHVDFS | (SEQ ID NO. 124) |
| SSESQRSRHGSVLSSNFTYHTSDGDALLLL. | |

As in Example 1 above, that the sequences before, between and after each list of transmembrane domain variants are the N′, intermediary and C′ intra or extracellular regions, respectively.

The sequences above were then used to generate coding sequences, as is known in the art, suitable for expression in the expression system, in this case yeast. The coding sequences were then shuffled and expressed to produce a library comprising a plurality of proteins each having SEQ ID NOs: 68, 77, 86, 88, 97, 106, and 115 with one transmembrane domain variant from each variant list in between the respective intracellular and extracellular domain.

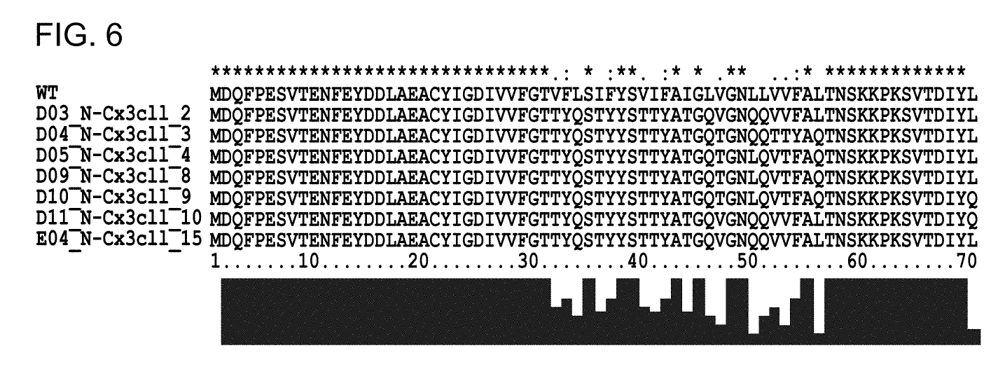

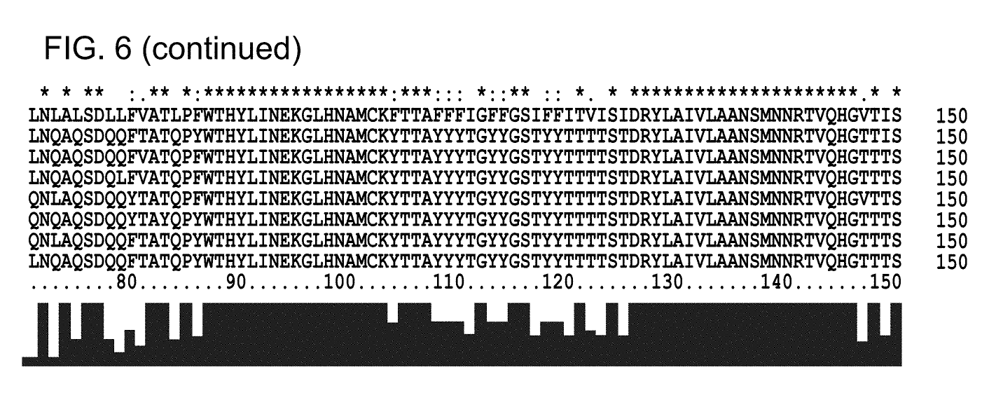

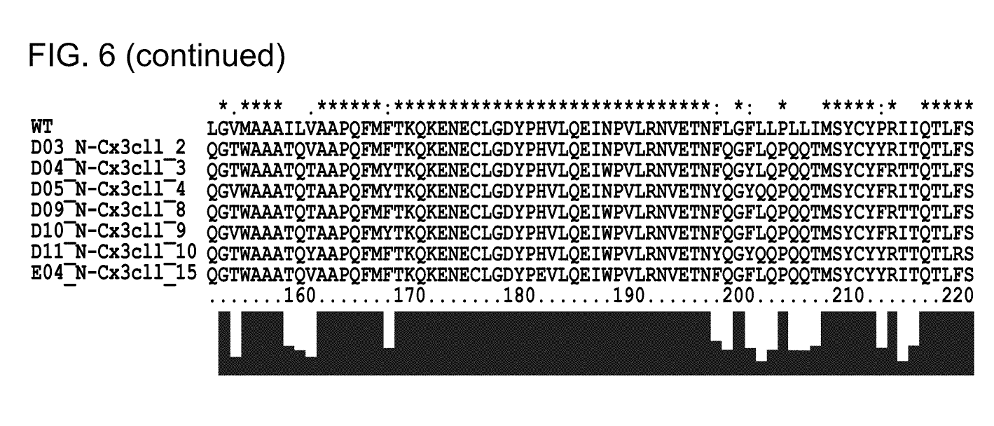

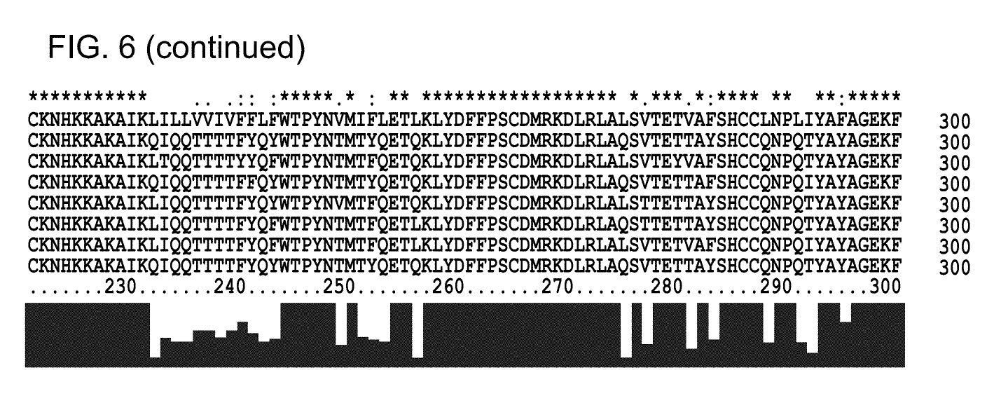

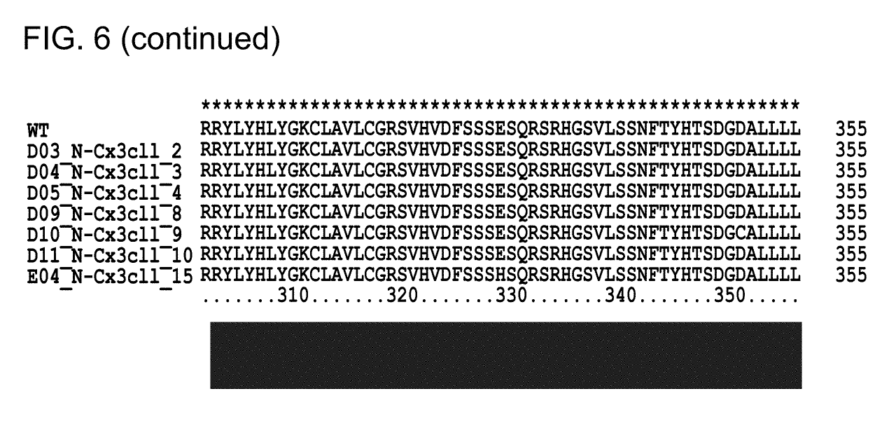

The library so produced was then assayed for CX3CR1 cognate ligand (CXCL1) binding in an aqueous medium, as described in Example 1. Ligand binding was detected and samples were then sequenced. Seven variants were sequenced. The results are shown in FIG. 6.

### Example 3: CCR3 Variants

The method of Example 1 was repeated for Chemokine Receptor Type 3 isoform 3.

| Name | pI | MW (Da) |
|---|---|---|
| WT | 8.87 | 43122.3 |
| MT | 8.78 | 43531.64 |

Replacing all or substantially all of the hydrophobic amino acids, L, I V, and F, with Q, T and Y (respectively) within the transmembrane domains results in the following sequence (lower line), aligned with the wild type (top line):

MPFGIRMLLRAHKPGRSEMTTSLDTVETFGTTSYYDDVGLLCEKADTRALMAQFVPPLYS
|||||||||||||||||||||||||||||||||||||||||||||||||||||||||*||
MPFGIRMLLRAHKPGRSEMTTSLDTVETFGTTSYYDDVGLLCEKADTRALMAQFVPPQYS
LVFTVGLLGNVVVVMILIKYRRLRIMTNIYLLNLAISDLLFLVTLPFWIHYVRGHNWVFG
***|*|**||****|***||||||||||*|**|*|*||*****|*|*|*|||||||||||
QTYTTGQQGNTTTTMTQTKYRRLRIMTNTYQQNQATSDQQYQTTQPYWTHYVRGHNWVFG
HGMCKLLSGFYHTGLYSEIFFIILLTIDRYLAIVHAVFALRARTVTFGVITSIVTWGLAV
||||||||||||||||||*******|*|||*|**||**|*||||||||**||**|||*|*
HGMCKLLSGFYHTGLYSETYYTTQQTTDRYQATTHATYAQRARTVTFGTTTSTTTWGQAT
LAALPEFIFYETEELFEETLCSALYPEDTVYSWRHFHTLRMTIFCLVLPLLVMAICYTGI
*||*||***|||||||||||||||||||||||||||||||||**|***|***||*||||*
QAAQPEYTYYETEELFEETLCSALYPEDTVYSWRHFHTLRMTTYCQTQPQQTMATCYTGT
IKTLLRCPSKKKYKAIRLIFVIMAVFFIFWTPYNVAILLSSYQSILFGNDCERSKHLDLV
*||||||||||||||||*****||*****|||||*|***|||||||||||||||||||**
TKTLLRCPSKKKYKAIRQTYTTMATYYTYWTPYNTATQQSSYQSILFGNDCERSKHLDQT
MLVTEVIAYSHCCMNPVIYAFVGERFRKYLRHFFHRHLLMHLGRYIPFLPSEKLERTSSV
|**||**|||||||||**||**||||||||||||||||||||||||||||||||||||||
MQTTETTAYSHCCMNPTTYAYTGERFRKYLRHFFHRHLLMHLGRYIPFLPSEKLERTSSV
SPSTAEPELSIVF (SEQ ID NO. 125)
|||||||||||||
SPSTAEPELSIVF (SEQ ID NO. 126)

Each of the predicted transmembrane regions have been underlined and exemplify a fully modified domain of the invention. Thus, for example, the invention includes a transmembrane domain comprising the underlined Amino Acids of SEQ ID NO: 126. Preferably the protein comprising TM1 herein includes one or more (e.g., all) of the extracellular and intracellular loop sequences of SEQ ID NO: 126 (the sequences which have not been underlined). In addition or alternatively, the protein comprising the TM1 herein includes one or more additional transmembrane regions (the underlined sequences) in SEQ ID NO: 126 or homologous sequences retaining one, two, three or, possibly four or more of the native V, L I and F amino acids, as set forth in SEQ ID NO: 125.

The native protein sequence for CCR3 was subjected to the method a second time (noting a difference in the N terminal sequence). The program output divided the native sequence into the extracellular and intracellular regions and selected 8 transmembrane domain variants for each transmembrane domain. The results are illustrated in the following table:

| | |
|---|---|
| MTTSLDTVETFGTTSYYDDVGLLCEKADTRALMA | (SEQ ID NO. 127) |
| TM1 Variants: | |
| QFVPPQYSQTFTTGQQGNVTVTMTQIKY | (SEQ ID NO. 128) |
| QFVPPQYSQTFTTGQQGNTTVTMTQIKY | (SEQ ID NO. 129) |
| QFVPPQYSQTYTTGQQGNTTVTMTQIKY | (SEQ ID NO. 130) |
| QFTPPQYSQTYTTGQQGNVTTTMTQIKY | (SEQ ID NO. 131) |
| QFTPPQYSQTYTTGQQGNTVTTMTQIKY | (SEQ ID NO. 132) |
| QFTPPQYSQTYTTGQQGNTTVTMTQIKY | (SEQ ID NO. 133) |
| QFTPPQYSQTYTTGQQGNTTTTMTQIKY | (SEQ ID NO. 134) |
| QYTPPQYSQTYTTGQQGNTTTTMTQTKY | (SEQ ID NO. 135) |
| RRLRIMTNIY | (SEQ ID NO. 136) |
| TM2 Variants: | |
| LLNQATSDQQFQVTQPFWIHY | (SEQ ID NO. 137) |
| LQNQAISDQLFQTTQPFWTHY | (SEQ ID NO. 138) |
| QQNLAISDQQFQTTQPFWTHY | (SEQ ID NO. 139) |
| QLNQAISDQQFQTTQPYWTHY | (SEQ ID NO. 140) |
| QQNLAISDQQYQVTQPYWTHY | (SEQ ID NO. 141) |
| LQNQATSDQLFQTTQPYWTHY | (SEQ ID NO. 142) |
| QQNQAISDQQYQVTQPYWTHY | (SEQ ID NO. 143) |
| QQNQATSDQQYQTTQPYWTHY | (SEQ ID NO. 144) |
| VRGHNWVFGHGMCK | (SEQ ID NO. 145) |
| TM3 Variants: | |
| LQSGFYHTGQYSETFFTTQQTT | (SEQ ID NO. 146) |
| QLSGFYHTGQYSETFFTTQQTT | (SEQ ID NO. 147) |
| QLSGFYHTGQYSETFYTTQQTT | (SEQ ID NO. 148) |
| QLSGFYHTGQYSETYFTTQQTT | (SEQ ID NO. 149) |
| QLSGYYHTGQYSETFFTTQQTT | (SEQ ID NO. 150) |
| QQSGFYHTGQYSETFFTTQQTT | (SEQ ID NO. 151) |
| QQSGFYHTGQYSETFYTTQQTT | (SEQ ID NO. 152) |
| QQSGYYHTGQYSETYYTTQQTT | (SEQ ID NO. 153) |
| DRYLAIVHAVFALRART | (SEQ ID NO. 154) |
| TM4 Variants: | |
| TTFGTTTSTVTWGQAVQAAQPEFIF | (SEQ ID NO. 155) |
| TTFGTTTSTTTWGQAVQAAQPEFIF | (SEQ ID NO. 156) |
| TTYGTTTSTTTWGQAVQAAQPEFIF | (SEQ ID NO. 157) |
| TTYGTTTSTTTWGQAVQAAQPEFTF | (SEQ ID NO. 158) |
| TTYGTTTSTTTWGQATQAAQPEFIF | (SEQ ID NO. 159) |
| TTFGTTTSTTTWGQATQAAQPEFIY | (SEQ ID NO. 160) |
| TTYGTTTSTTTWGQATQAAQPEFIY | (SEQ ID NO. 161) |
| TTYGTTTSTTTWGQATQAAQPEYTY | (SEQ ID NO. 162) |
| YETEELFEETLCSALYPEDTVYSWRHFHTLRM | (SEQ ID NO. 163) |
| TM5 Variants: | |
| TIFCQVQPQQTMATCYTGTT | (SEQ ID NO. 164) |
| TIFCQTQPQQVMATCYTGTT | (SEQ ID NO. 165) |
| TIFCQTQPQQTMATCYTGIT | (SEQ ID NO. 166) |
| TIFCQTQPQQTMATCYTGTI | (SEQ ID NO. 167) |
| TTFCQVQPQQVMATCYTGTT | (SEQ ID NO. 168) |
| TIYCQVQPQQVMATCYTGTT | (SEQ ID NO. 169) |
| TIFCQTQPQQTMATCYTGTT | (SEQ ID NO. 170) |
| TTYCQTQPQQTMATCYTGTT | (SEQ ID NO. 171) |
| KTLLRCPSKKKYKAIR | (SEQ ID NO. 172) |
| TM 6 Variant: | |
| QTYTTMATYYTYWTPYNTATQQSSY | (SEQ ID NO. 173) |
| QSILFGNDCERSKHLDL | (SEQ ID NO. 174) |
| TM7 Variants: | |
| VMQVTEVTAYSHCCMNPVTYAFTG | (SEQ ID NO. 175) |
| VMQVTEVTAYSHCCMNPTTYAYVG | (SEQ ID NO. 176) |
| VMLTTEVTAYSHCCMNPTTYAFTG | (SEQ ID NO. 177) |
| VMQVTETTAYSHCCMNPVTYAYTG | (SEQ ID NO. 178) |
| TMQVTETIAYSHCCMNPTTYAFTG | (SEQ ID NO. 179) |
| TMQVTETTAYSHCCMNPTTYAFVG | (SEQ ID NO. 180) |
| VMQTTETIAYSHCCMNPTTYAYTG | (SEQ ID NO. 181) |
| TMQTTETTAYSHCCMNPTTYAYTG | (SEQ ID NO. 182) |
| ERFRKYLRHFFHRHLLMHLGRYIPFLPSEKLE | (SEQ ID NO: 183) |
| RTSSVSPSTAEPELSIVF. | |

As in Example 1 above, the sequences before, between and after each list of transmembrane domain variants are the N′, intermediary and C′ intra or extracellular regions, respectively.

The sequences above were then used to generate coding sequences, as is known in the art, suitable for expression in the expression system, in this case yeast. The coding sequences were then shuffled and expressed to produce a library comprising a plurality of proteins each having SEQ ID NOs: 127, 136, 145, 154, 163, 172, 174 and 183 with one transmembrane domain variant from each variant list in between the respective intracellular and extracellular domain.

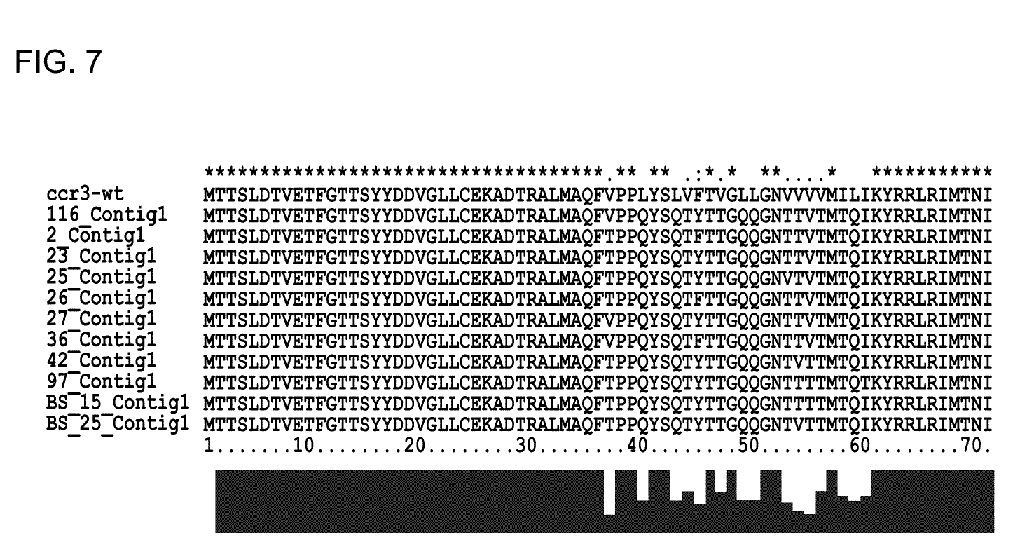

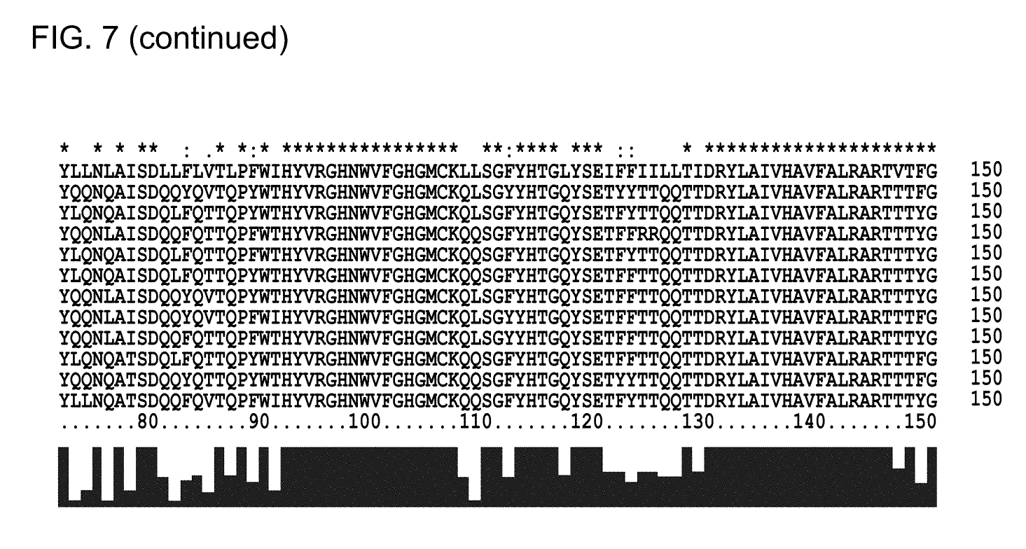

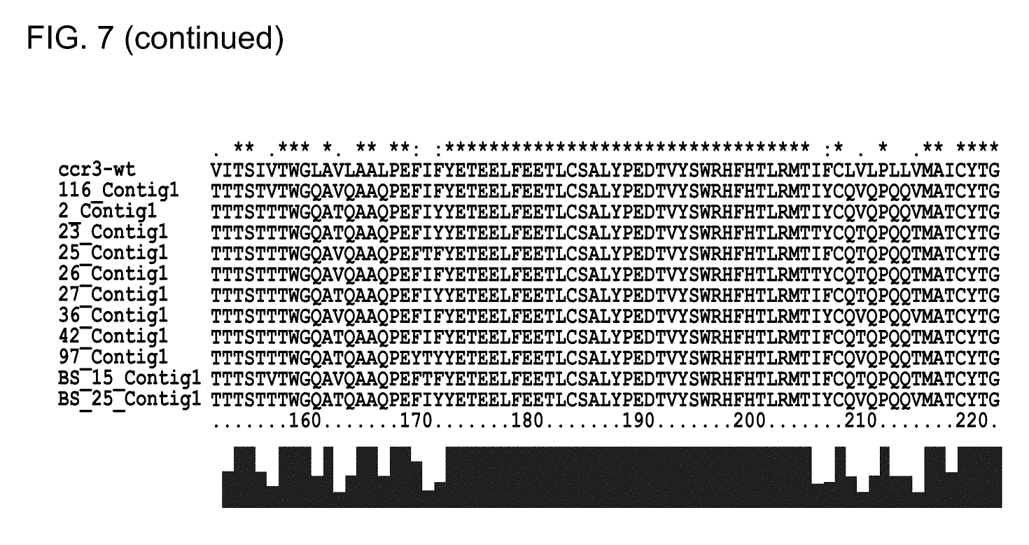

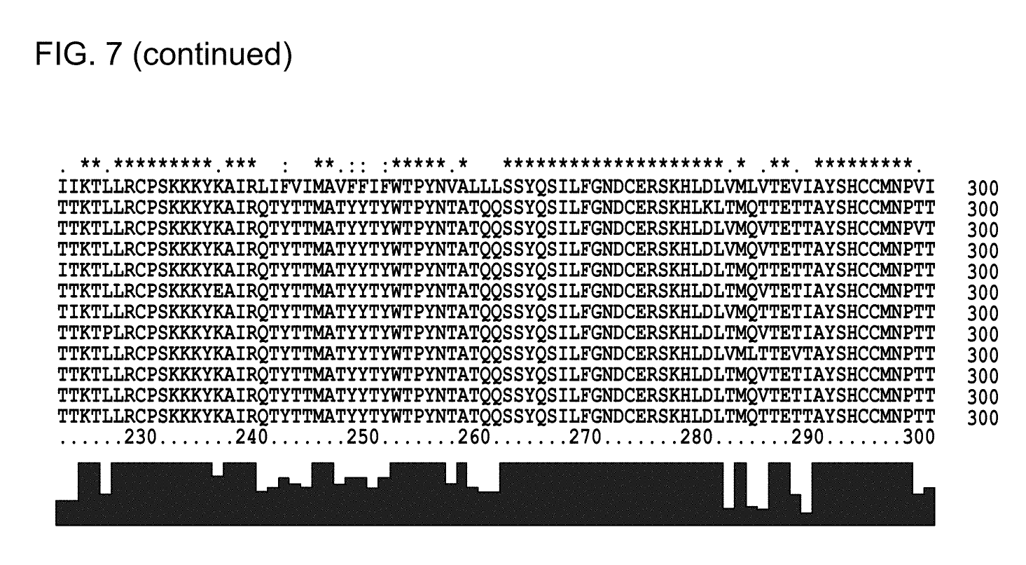

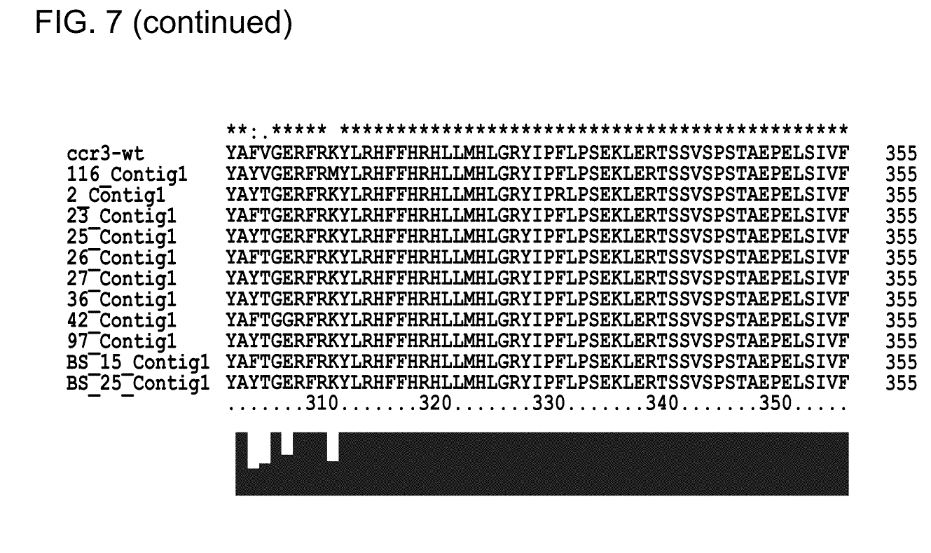

The library so produced was then assayed for CCR3 cognate ligand, CCL3, binding in an aqueous medium, as described in Example 1. Ligand binding was detected and samples were then sequenced. Eleven variants were sequenced. The results are shown in FIG. 7.

### Example 4: CCR5 Variants

The method of Example 1 was repeated for Chemokine Receptor Type 5 isoform 3.

| Name | pI | MW (Da) |
|---|---|---|
| WT | 9.21 | 40524.05 |
| MT | 9.06 | 41058.3 |

Replacing all or substantially all of the hydrophobic amino acids, L, I V, and F, with Q, T and Y (respectively) within the transmembrane domains results in the following sequence (lower line), aligned with the wild type (top line):

MDYQVSSPIYDINYYTSEPCQKINVKQIAARLLPPLYSLVFIFGFVGNMLVILILINCKR
|||||||||||||||||||||||||||||||||||*||*****|**|||*******||||
MDYQVSSPIYDINYYTSEPCQKINVKQIAARLLPPQYSQTYTYGYTGNMQTTQTQTNCKR
LKSMTDIYLLNLAISDLFFLLTVPFWAHYAAAQWDFGNTMCQLLTGLYFIGFFSGIFFII
||||||*|**|*|*||*****|*|*|||||||||||||||||**||*|**|**||*****
LKSMTDTYQQNQATSDQYYQQTTPYWAHYAAAQWDFGNTMCQQQTGQYYTGYYSGTYYTT
LLTIDRYLAVVHAVFALKARTVTFGVVTSVITWVVAVFASLPGIIFTRSQKEGLHYTCSS
**|*|||||||||||||||||||*|**||**||**|**||*||***||||||||||||||
QQTTDRYLAVVHAVFALKARTVTYGTTTSTTTWTTATYASQPGTTYTRSQKEGLHYTCSS
HFPYSQYQFWKNFQTLKIVILGLVLPLLVMVICYSGILKTLLRCRNEKKRHRAVRLIFTI
|||||||||||||||||****|***|***|**||||**||*||||||||||||||***|*
HFPYSQYQFWKNFQTLKTTTQGQTQPQQTMTTCYSGTQKTQLRCRNEKKRHRAVRQTYTT
MIVYFLFWAPYNIVLLLNTFQEFFGLNNCSSSNRLDQAMQVTETLGMTHCCINPIIYAFV
|**|***|||||*****||||||||||||||||||||||||||||||||||*||**||**
MTTYYQYWAPYNTTQQQNTFQEFFGLNNCSSSNRLDQAMQVTETLGMTHCCTNPTTYAYT
GEKFRNYLLVFFQKHIAKRFCKCCSIFQQEAPERASSVYTRSTGEQEISVGL (SEQ ID NO. 184)
|||*|||*****||||||||||||||||||||||||||||||||||||||||
GEKYRNYQQTYYQKHIAKRFCKCCSIFQQEAPERASSVYTRSTGEQEISVGL. (SEQ ID NO. 185)

Each of the predicted transmembrane regions has been underlined and exemplified a fully modified domain of the invention. Thus, for example, the invention includes a transmembrane domain comprising the underlined Amino Acids of SEQ ID NO: 185. Preferably the protein comprising TM1 herein includes one or more (e.g., all) of the extracellular and intracellular loop sequences of SEQ ID NO: 185 (the sequences which have not been underlined). In addition or alternatively, the protein comprising the TM1 herein includes one or more additional transmembrane regions (the underlined sequences) in SEQ ID NO: 185 or homologous sequences retaining one, two, three or, possibly four or more of the native V, L I and F amino acids, as set forth in SEQ ID NO: 184.

The native protein sequence for CCR5 was subjected to the method a second time (noting a difference in the N terminal sequence). The program output divided the native sequence into the extracellular and intracellular regions and selected 8 transmembrane domain variants for each transmembrane domain. The results are illustrated in the following table:

| | |
|---|---|
| MDYQVSSPIYDINYYTSEPCQKINVKQIAA | (SEQ ID NO. 186) |
| TM1 Variants: | |
| RLQPPQYSQTFTFGFTGNMQVTQTQINC | (SEQ ID NO. 187) |
| RLQPPQYSQTFTFGYTGNMQVTQTQINC | (SEQ ID NO. 188) |
| RQQPPQYSQTFTFGFTGNMQTTQTQINC | (SEQ ID NO. 189) |
| RQQPPQYSQTFTYGFTGNMQTTQTQINC | (SEQ ID NO. 190) |
| RQQPPQYSQTYTFGFTGNMQTTQTQINC | (SEQ ID NO. 191) |
| RQQPPQYSQTFTFGYTGNMQTTQTQINC | (SEQ ID NO. 192) |
| RQQPPQYSQTYTFGYTGNMQTTQTQINC | (SEQ ID NO. 193) |
| RQQPPQYSQTYTYGYTGNMQTTQTQTNC | (SEQ ID NO. 194) |
| KRLKSMTDIY | (SEQ ID NO. 195) |
| TM2 Variants: | |
| LQNQAISDQFFQQTVPFWAHY | (SEQ ID NO. 196) |
| LQNQAISDQFFQQTTPFWAHY | (SEQ ID NO. 197) |
| LQNQAISDQFFQQTTPYWAHY | (SEQ ID NO. 198) |
| LQNQAISDQFYQQTTPYWAHY | (SEQ ID NO. 199) |
| LQNQAISDQYFQQTTPYWAHY | (SEQ ID NO. 200) |
| LQNQATSDQFFQQTTPYWAHY | (SEQ ID NO. 201) |
| LQNQAISDQYYQQTTPYWAHY | (SEQ ID NO. 202) |
| QQNQATSDQYYQQTTPYWAHY | (SEQ ID NO. 203) |
| AAAQWDFGNTMCQ | (SEQ ID NO. 204) |
| TM3 Variants: | |
| QQTGQYFTGYYSGTYYTTQQTT | (SEQ ID NO. 205) |
| QQTGQYYTGYYSGTYYTTQQTT | (SEQ ID NO. 206) |
| DRYLAVVHAVFALKART | (SEQ ID NO. 207) |
| TM4 Variant: | |
| TTYGTTTSTTTWTTATYASQPGTTY | (SEQ ID NO. 208) |
| TRSQKEGLHYTCSSHFPYSQYQFWKNFQTLKI | (SEQ ID NO. 209) |
| TM5 Variants: | |
| VIQGQVQPQQVMVTCYSGIQ | (SEQ ID NO. 210) |
| VIQGQVQPQQVMTTCYSGIQ | (SEQ ID NO. 211) |
| VIQGQVQPQQTMTTCYSGIQ | (SEQ ID NO. 212) |
| VTQGQVQPQQTMVTCYSGTQ | (SEQ ID NO. 213) |
| TIQGQVQPQQVMTTCYSGTQ | (SEQ ID NO. 214) |
| TIQGQVQPQQTMVTCYSGTQ | (SEQ ID NO. 215) |
| TTQGQVQPQQVMTTCYSGTQ | (SEQ ID NO. 216) |
| TTQGQTQPQQTMTTCYSGTQ | (SEQ ID NO. 217) |
| KTLLRCRNEKKRHRAVR | (SEQ ID NO. 218) |
| TM6 Variants: | |
| QTFTTMTTYYQFWAPYNIVQQLNTF | (SEQ ID NO. 219) |
| QTFTTMTTYYQFWAPYNTVQQLNTF | (SEQ ID NO. 220) |
| QTFTTMTTYYQYWAPYNTVQQLNTF | (SEQ ID NO. 221) |
| QTFTTMTTYYQYWAPYNTVQQQNTF | (SEQ ID NO. 222) |
| QTYTTMTTYYQYWAPYNTVQQLNTF | (SEQ ID NO. 223) |
| QTFTTMTTYYQYWAPYNTTQQLNTF | (SEQ ID NO. 224) |
| QTYTTMTTYYQYWAPYNTVQQQNTF | (SEQ ID NO. 225) |
| QTYTTMTTYYQYWAPYNTTQQQNTY | (SEQ ID NO. 225) |
| QEFFGLNNCSSSNRLDQ | (SEQ ID NO. 226) |
| TM7 Variants: | |
| AMQVTETQGMTHCCINPIIYAFVG | (SEQ ID NO. 227) |
| AMQVTETLGMTHCCTNPIIYAFTG | (SEQ ID NO. 228) |
| AMQVTETQGMTHCCINPTIYAYVG | (SEQ ID NO. 229) |
| AMQTTETQGMTHCCINPITYAFTG | (SEQ ID NO. 230) |
| AMQTTETQGMTHCCINPTIYAFTG | (SEQ ID NO. 231) |
| AMQVTETQGMTHCCTNPTIYAYVG | (SEQ ID NO. 232) |
| AMQTTETQGMTHCCINPTTYAYVG | (SEQ ID NO. 233) |
| AMQTTETQGMTHCCTNPTTYAYTG | (SEQ ID NO. 234) |
| EKFRNYLLVFFQKHIAKRFCKCCSIFQQEAPER | (SEQ ID NO. 235) |
| ASSVYTRSTGEQEISVGL. | |

As in Example 1 above, the sequences before, between and after each list of transmembrane domain variants are the N′, intermediary and C′ intra or extracellular regions, respectively.

The sequences above were then used to generate coding sequences, as is known in the art, suitable for expression in the expression system, in this case yeast. The coding sequences were then shuffled and expressed to produce a library comprising a plurality of proteins each having SEQ ID NOs, 186, 195, 204, 207, 209, 218, 226, and 235 with one transmembrane domain variant from each variant list in between the respective intracellular and extracellular domain.

The library so produced was then assayed for CCR5 cognate ligand, CCL5, binding in an aqueous medium, as described in Example 1. Ligand binding was detected and samples were then sequenced. One variant was sequenced. The results are shown in FIG. 8.

### Example 5: CXCR3 Variants

The method of Example 1 was repeated for the CXC chemokine receptor type 3 isoform 2. Replacing all or substantially all of the hydrophobic amino acids, L, I V, and F, with Q, T and Y (respectively) within the transmembrane domains results in the following sequence (SEQ ID NO: 325, lower line), aligned with the wild type (SEQ ID NO: 324, top line):

MELRKYGPGRLAGTVIGGAAQSKSQTKSDSITKEFLPGLYTAPSSPFPPSQVSDHQVLND
||||||||||||||||||||||||||||||||||||||||||||||||||||||||||||
MELRKYGPGRLAGTVIGGAAQSKSQTKSDSITKEFLPGLYTAPSSPFPPSQVSDHQVLND
AEVAALLENFSSSYDYGENESDSCCTSPPCPQDFSLNFDRAFLPALYSLLFLLGLLGNGA
|||||||||||||||||||||||||||||||||||||||||||||*||*****|**||||
AEVAALLENFSSSYDYGENESDSCCTSPPCPQDFSLNFDRAFLPAQYSQQYQQGQQGNGA
VAAVLLSRRTALSSTDTFLLHLAVADTLLVLTLPLWAVDAAVQWVFGSGLCKVAGALFNI
*||***|||||||||||***|*|*|||****|*|*||*||||||||||||||||||**|*
TAATQQSRRTALSSTDTYQQHQATADTQQTQTQPQWATDAAVQWVFGSGLCKVAGAQYNT
NFYAGALLLACISFDRYLNIVHATQLYRRGPPARVTLTCLAVWGLCLLFALPDFIFLSAH
|*||||***||*|*||||||||||||||||||||*|*||*|*||*|***|*||****|||
NYYAGAQQQACTSYDRYLNIVHATQLYRRGPPARTTQTCQATWGQCQQYAQPDYTYQSAH
HDERLNATHCQYNFPQVGRTALRVLQLVAGFLLPLLVMAYCYAHILAVLLVSRGQRRLRA
|||||||||||||||||||||||||||*||***|***|||||||**|***||||||||||
HDERLNATHCQYNFPQVGRTALRVLQLTAGYQQPQQTMAYCYAHTQATQQVSRGQRRLRA
MRLVVVVVVAFALCWTPYHLVVLVDILMDLGALARNCGRESRVDVAKSVTSGLGYMHCCL
||*******|*|*||||||***||||||||||||||||||||||||||||||*||||||*
MRQTTTTTTAYAQCWTPYHQTTLVDILMDLGALARNCGRESRVDVAKSVTSGQGYMHCCQ
NPLLYAFVGVKFRERMWMLLLRLGCPNQRGLQRQPSSSRRDSSWSETSEASYSGL
||**||**|*|||||||||||||||||||||||||||||||||||||||||||||
NPQQYAYTGTKFRERMWMLLLRLGCPNQRGLQRQPSSSRRDSSWSETSEASYSGL

Each of the predicted transmembrane regions has been underlined and exemplified a fully modified domain of the invention. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in SEQ ID NO: 325 or homologous sequences retaining one, two, three or, possibly four or more of the native V, L I and F amino acids, as set forth in SEQ ID NO: 324.

As discussed above, the native protein sequence for CXCR3 was subjected to the method. The program output divided the native sequence into the extracellular and intracellular regions and selected 8 transmembrane domain variants for each transmembrane domain. The results are illustrated in the following table:

| | |
|---|---|
| MVLEVSDHQVLNDAEVAALLENFSSSYDYGE | (SEQ ID NO. 235) |
| NESDSCCTSPPCPQDFSLNFDR | |
| TM 1 Variants: | |
| AFLPALYSQQFQQGQQGNGAVAATQLS | (SEQ ID NO. 236) |
| AFQPALYSQQFQQGQQGNGAVAAVQQS | (SEQ ID NO. 237) |
| AFQPAQYSQQFLQGQQGNGAVAATQQS | (SEQ ID NO. 238) |
| AYQPALYSLQYQQGQQGNGATAAVQQS | (SEQ ID NO. 239) |
| AYQPALYSQLFQQGQQGNGATAATQQS | (SEQ ID NO. 240) |
| AFQPALYSLQYQQGQQGNGATAATQQS | (SEQ ID NO. 241) |
| AYQPAQYSLQYQQGQQGNGATAAVQQS | (SEQ ID NO. 242) |
| AYQPAQYSQQYQQGQQGNGATAATQQS | (SEQ ID NO. 243) |
| RRTALSSTD | (SEQ ID NO. 244) |
| TM 2 Variants: | |
| TFLQHLAVADTQQVQTLPQWA | (SEQ ID NO. 245) |
| TFLQHQAVADTQLVQTQPQWA | (SEQ ID NO.: 246) |
| TFQQHLAVADTQQVQTQPQWA | (SEQ ID NO.: 247) |
| TYLQHQAVADTQQVQTQPQWA | (SEQ ID NO.: 248) |
| TYQLHQAVADTQQVQTQPQWA | (SEQ ID NO.: 249) |
| TYQQHLAVADTQQVQTQPQWA | (SEQ ID NO.: 250) |
| TYQQHQAVADTQQVQTQPQWA | (SEQ ID NO.: 251) |
| TYQQHQATADTQQTQTQPQWA | (SEQ ID NO.: 252) |
| VDAAVQWVFGSGLCK | (SEQ ID NO.: 253) |
| TM 3 Variants: | |
| TAGAQYNTNFYAGAQQQACISF | (SEQ ID NO.: 254) |
| TAGAQYNTNFYAGAQLQACTSF | (SEQ ID NO.: 255) |
| TAGAQYNTNFYAGAQQLACTSF | (SEQ ID NO.: 256) |
| TAGAQFNTNYYAGAQQQACISF | (SEQ ID NO.: 257) |
| TAGAQYNTNYYAGAQQQACISF | (SEQ ID NO.: 258) |
| TAGAQYNTNYYAGAQLQACTSF | (SEQ ID NO.: 259) |
| TAGAQYNTNYYAGAQQLACTSF | (SEQ ID NO.: 260) |
| TAGAQYNTNYYAGAQQQACTSY | (SEQ ID NO.: 261) |
| DRYLNIVHATQLYRRGPPARVT | (SEQ ID NO.: 262) |
| TM 4 Variants: | |
| LTCQAVWGQCQQFAQPDFIF | (SEQ ID NO.: 263) |
| QTCQAVWGQCQQFAQPDFIF | (SEQ ID NO.: 264) |
| QTCQATWGQCQQFAQPDFIF | (SEQ ID NO.: 265) |
| QTCQATWGQCQQYAQPDFIF | (SEQ ID NO.: 266) |
| QTCQATWGQCQQFAQPDFTF | (SEQ ID NO.: 267) |
| QTCQATWGQCQQFAQPDYIF | (SEQ ID NO.: 268) |
| QTCQATWGQCQQYAQPDYIF | (SEQ ID NO.: 269) |
| QTCQATWGQCQQYAQPDYTY | (SEQ ID NO.: 270) |
| LSAHHDERLNATHCQYNFPQVGR | (SEQ ID NO.: 271) |
| TM 5 Variant: | |
| TAQRTQQQTAGYQQPQQTMAY | (SEQ ID NO.: 272) |
| CYAHILAVLLVSRGQRRLRAMR | (SEQ ID NO.: 273) |
| TM 6 Variants: | |
| QVTTTTVAFAQCWTPYHQVVQV | (SEQ ID NO.: 274) |
| QVTTTTVAFAQCWTPYHQTVQV | (SEQ ID NO.: 275) |
| QVTTTTTAFAQCWTPYHQTVQV | (SEQ ID NO.: 276) |
| QVTTTTTAYAQCWTPYHQTVQV | (SEQ ID NO.: 277) |
| QVTTTTTAFAQCWTPYHQTTQV | (SEQ ID NO.: 278) |
| QTTTTTVAFAQCWTPYHQTTQV | (SEQ ID NO.: 279) |
| QVTTTTTAYAQCWTPYHQTTQV | (SEQ ID NO.: 280) |
| QTTTTTTAYAQCWTPYHQTTQT | (SEQ ID NO.: 281) |
| DILMDLGALARNCGRESRVDV | (SEQ ID NO.: 282) |
| TM 7 Variants: | |
| AKSVTSGQGYMHCCLNPLQYAFV | (SEQ ID NO.: 283) |
| AKSVTSGQGYMHCCLNPQLYAFT | (SEQ ID NO.: 284) |
| AKSVTSGQGYMHCCLNPLQYAFT | (SEQ ID NO.: 285) |
| AKSTTSGQGYMHCCLNPQQYAFV | (SEQ ID NO.: 286) |
| AKSTTSGQGYMHCCQNPLQYAFV | (SEQ ID NO.: 287) |
| AKSTTSGQGYMHCCQNPQLYAFV | (SEQ ID NO.: 288) |
| AKSTTSGQGYMHCCQNPLQYAFT | (SEQ ID NO.: 289) |
| AKSTTSGQGYMHCCQNPQQYAYT | (SEQ ID NO.: 290) |
| GVKFRERMWMLLLRLGCPNQRGLQRQPSSSR | (SEQ ID NO.: 291) |
| RDSSWSETSEASYSGL. | |

The sequences above can be used to generate coding sequences, as is known in the art, suitable for expression in the expression system, in this case yeast. The coding sequences were then shuffled and expressed to produce a library comprising a plurality of proteins each having the intracellular and extracellular loops with one transmembrane domain variant from each variant list in between the respective intracellular and extracellular domain.

The library so produced can then be assayed for cognate ligand binding in an aqueous medium, as described in Example 1.

### Example 6: CCR-1 C-C Chemokine Receptor Type 1

Example 1 was repeated for the title protein.

| Name | pI | MW (Da) |
|---|---|---|
| WT | 8.38 | 41172.64 |
| MT | 8.31 | 41583.78 |

Replacing all or substantially all of the hydrophobic amino acids, L, I V, and F, with Q, T and Y (respectively) within the transmembrane domains results in the following sequence (lower line SEQ ID NO: 293), aligned with the wild type (top line SEQ ID NO: 292):

METPNTTEDYDTTTEFDYGDATPCQKVNERAFGAQLLPPLYSLVFVIGLVGNILVVLVLV
||||||||||||||||||||||||||||||||||||*||*||*****|**||*******|
METPNTTEDYDTTTEFDYGDATPCQKVNERAFGAQLQPPQYSQTYTTGQTGNTQTTQTQV
QYKRLKNMTSIYLLNLAISDLLFLFTLPFWIDYKLKDDWVFGDAMCKILSGFYYTGLYSE
||||||||||*|**|*|*||*****|*|*|*||||||||||||||||**||*||||*|||
QYKRLKNMTSTYQQNQATSDQQYQYTQPYWTDYKLKDDWVFGDAMCKTQSGYYYTGQYSE
IFFIILLTIDRYLAIVHAVFALRARTVTFGVITSIIIWALAILASMPGLYFSKTQWEFTH
*******|||||||||||||||||||*|*|**||***||*|**|||||*|||||||||||
TYYTTQQTIDRYLAIVHAVFALRARTTTYGTTTSTTTWAQATQASMPGQYFSKTQWEFTH
HTCSLHFPHESLREWKLFQALKLNLFGLVLPLLVMIICYTGIIKILLRRPNEKKSKAVRL
||||||||||||||||||||||||**|***|***|**||||**|***||||||||||||*
HTCSLHFPHESLREWKLFQALKLNQYGQTQPQQTMTTCYTGTTKTQQRRPNEKKSKAVRQ
IFVIMIIFFLFWTPYNLTILISVFQDFLFTHECEQSRHLDLAVQVTEVIAYTHCCVNPVI
****|******|||||*|***|||||||||||||||||||||*|*||**||||||*||**
TYTTMTTYYQYWTPYNQTTQTSVFQDFLFTHECEQSRHLDLATQTTETTAYTHCCTNPTT
YAFVGERFRKYLRQLFHRRVAVHLVKWLPFLSVDRLERVSSTSPSTGEHELSAGF
||**|||||||||||||||||||||||||||||||||||||||||||||||||||
YAYTGERFRKYLRQLFHRRVAVHLVKWLPFLSVDRLERVSSTSPSTGEHELSAGF

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native V, L I and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 7: CCR-2 C-C Chemokine Receptor Type 2 Isoform A

Example 1 was repeated for the title protein. Replacing each of the hydrophobic amino acids, L, I V, and F, with Q, T and Y (respectively) within the transmembrane domains results in the following sequence (lower line SEQ ID NO.:295), aligned with the wild type (top line SEQ ID NO: 294):

MLSTSRSRFIRNTNESGEEVTTFFDYDYGAPCHKFDVKQIGAQLLPPLYSLVFIFGFVGN
||||||||||||||||||||||||||||||||||||||||||||*||*||*****|**||
MLSTSRSRFIRNTNESGEEVTTFFDYDYGAPCHKFDVKQIGAQLQPPQYSQTYTYGYTGN
MLVVLILINCKKLKCLTDIYLLNLAISDLLFLITLPLWAHSAANEWVFGNAMCKLFTGLY
|******|||||||||||||**|*|*||*****|*|*|||||||||||||||||||||*|
MQTTQTQINCKKLKCLTDIYQQNQATSDQQYQTTQPQWAHSAANEWVFGNAMCKLFTGQY
HIGYFGGIFFIILLTIDRYLAIVHAVFALKARTVTFGVVTSVITWLVAVFASVPGIIFTK
|*||*||*******|*|||||||||||||||||||*|**||**||**|**||*||***||
HTGYYGGTYYTTQQTTDRYLAIVHAVFALKARTVTYGTTTSTTTWQTATYASTPGTTYTK
CQKEDSVYVCGPYFPRGWNNFHTIMRNILGLVLPLLIMVICYSGILKTLLRCRNEKKRHR
|||||||||||||||||||||||||||**|***|***|**||||**||**||||||||||
CQKEDSVYVCGPYFPRGWNNFHTIMRNTQGQTQPQQTMTTCYSGTQKTQQRCRNEKKRHR
AVRVIFTIMIVYFLFWTPYNIVILLNTFQEFFGLSNCESTSQLDQATQVTETLGMTHCCI
|*|***|*|**|***|||||****||||||||||||||||||||||||||||*||||||*
TRTTYTTMTTYYQYWTPYNTTTQLNTFQEFFGLSNCESTSQLDQATQVTETQGMTHCCT
NPIIYAFVGEKFRSLFHIALGCRIAPLQKPVCGGPGVRPGKNVKVTTQGLLDGRGKGKSI
||**||**||||||||||||||||||||||||||||||||||||||||||||||||||||
NPTTYAYTGEKFRSLFHIALGCRIAPLQKPVCGGPGVRPGKNVKVTTQGLLDGRGKGKSI
GRAPEASLQDKEGA
||||||||||||||
GRAPEASLQDKEGA

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native V, L I and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 8: CCR-4 C-C Chemokine Receptor Type 4

Example 1 was repeated for the title protein. Replacing all or substantially all of the hydrophobic amino acids, L, I V, and F, with Q, T and Y (respectively) within the transmembrane domains results in the following sequence (lower line SEQ ID NO: 297), aligned with the wild type (top line SEQ ID NO: 296):

MNPTDIADTTLDESIYSNYYLYESIPKPCTKEGIKAFGELFLPPLYSLVFVFGLLGNSVV
|||||||||||||||||||||||||||||||||||||||||||||||*****|**|||**
MNPTDIADTTLDESIYSNYYLYESIPKPCTKEGIKAFGELFLPPLYSQTYTYGQQGNSTT
VLVLFKYKRLRSMTDVYLLNLAISDLLFVFSLPFWGYYAADQWVFGLGLCKMISNMYLVG
*****||||||||||*|**|*|*||*****|*|*||||||||||||||||||*||||**|
TQTQYKYKRLRSMTDTYQQNQATSDQQYTYSQPYWGYYAADQWVFGLGLCKMTSWMYQTG
FYSGIFFVMLMSIDRYLAIVHAVFSLRARTLTYGVITSLATWSVAVFASLPGFLFSTCYT
*|||****|*||||||||||||||||||||*|||**||*||||*|**||*||***|||||
YYSGTYYTMQMSIDRYLAIVHAVFSLRARTQTYGTTTSQATWSTATYASQPGYQYSTCYT
ERNHTYCKTKYSLNSTTWKVLSSLEINILGLVIPLGIMLFCYSMIIRTLQHCKNEKKNKA
|||||||||||||||||||||||||*|**|***|*|*|**||||**||||||||||||||
ERNHTYCKTKYSLNSTTWKVLSSLETNTQGQTTPQGTMQYCYSMTTRTLQHCKNEKKNKA
VKMIFAVVVLFLGFWTPYNIVLFLETLVELEVLQDCTFERYLDYAIQATETLAFVHCCLN
|||**|******|*|||||*****|||||||||||||||||||||||||||*|**|||*|
VKMTYATTTQYQGYWTPYNTTQYQETLVELEVLQDCTFERYLDYAIQATETQAYTHCCQN
PIIYFFLGEKFRKYILQLFKTCRGLFVLCQYCGLLQTYSADTPSSSYTQSTMDHDLHDAL
|**|***|||||||||||||||||||||||||||||||||||||||||||||||||||||
PTTYYYQGEKFRKYILQLFKTCRGLFVLCQYCGLLQTYSADTPSSSYTQSTMDHDLHDAL

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native V, L I and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 9: CCR-6 C-C Chemokine Receptor Type 6

Example 1 was repeated for the title protein. Replacing all or substantially all of the hydrophobic amino acids, L, I V, and F, with Q, T and Y (respectively) within the transmembrane domains results in the following sequence (lower line SEQ ID NO: 299), aligned with the wild type (top line SEQ ID NO: 298):

MSGESMNFSDVFDSSEDYFVSVNTSYYSVDSEMLLCSLQEVRQFSRLFVPIAYSLICVFG
||||||||||||||||||||||||||||||||||||||||||||||||||*|||**|**|
MSGESMNFSDVFDSSEDYFVSVNTSYYSVDSEMLLCSLQEVRQFSRLFVPTAYSQTCTYG
LLGNILVVITFAFYKKARSMTDVYLLNMAIADILFVLTLPFWAVSHATGAWVFSNATCKL
**||*****|*|*|||||||||||**|||*||*****|*|*||*||||||||||||||||
QQGNTQTTTTYAYYKKARSMTDVYQQNMATADTQYTQTQPYWATSHATGAWVFSNATCKL
LKGIYAINFNCGMLLLTCISMDRYIAIVQATKSFRLRSRTLPRSKIICLVVWGLSVIISS
|||*||*|*||||***||*|||||*||||||||||||||||||||**|***||*|***||
LKGTYATNYNCGMQQQTCTSMDRYTAIVQATKSFRLRSRTLPRSKTTCQTTWGQSTTTSS
STFVFNQKYNTQGSDVCEPKYQTVSEPIRWKLLMLGLELLFGFFIPLMFMIFCYTFIVKT
||***||||||||||||||||||||||||||||||||||**|***|*|*|**|||***||
STYTYNQKYNTQGSDVCEPKYQTVSEPIRWKLLMLGLELQYGYYTPQMYMTYCYTYTTKT
LVQAQNSKRHKAIRVIIAVVLVFLACQIPHNMVLLVTAANLGKMNRSCQSEKLIGYTKTV
**||||||||||||***|******|||*||||****||||||||||||||||||||||||
QTQAQNSKRHKAIRTTTATTQTYQACQTPHNMTQQTTAANLGKMNRSCQSEKLIGYTKTV
TEVLAFLHCCLNPVLYAFIGQKFRNYFLKILKDLWCVRRKYKSSGFSCAGRYSENISRQT
||**|**|||*||**||**|||||||||||||||||||||||||||||||||||||||||
TETQAYQHCCQNPTQYAYTGQKFRNYFLKILKDLWCVRRKYKSSGFSCAGRYSENISRQT
SETADNDNASSFTM
||||||||||||||
SETADNDNASSFTM

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native L, I, V and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 10: CCR-7 C-C Chemokine Receptor Type 7 Precursor

Example 1 was repeated for the title protein. Replacing all or substantially all of the hydrophobic amino acids, L, I V, and F, with Q, T and Y (respectively) within the transmembrane domains results in the following sequence (lower line SEQ ID NO: 301), aligned with the wild type (top line SEQ ID NO: 300):

MDLGKPMKSVLVVALLVIFQVCLCQDEVTDDYIGDNTTVDYTLFESLCSKKDVRNFKAWF
||||||||||||||||||||||||||||||||||||||||||||||||||||||||||||
MDLGKPMKSVLVVALLVIFQVCLCQDEVTDDYIGDNTTVDYTLFESLCSKKDVRNFKAWF
LPIMYSIICFVGLLGNGLVVLTYIYFKRLKTMTDTYLLNLAVADILFLLTLPFWAYSAAK
||*|||**|**|**|||****||*||||||||||||**|*|*||*****|*|*|||||||
LPTMYSTTCYTGQQGNGQTTQTYTYFKRLKTMTDTYQQNQATADTQYQQTQPYWAYSAAK
SWVFGVHFCKLIFAIYKMSFFSGMLLLLCISIDRYVAIVQAVSAHRHRARVLLISKLSCV
||||||||||***|*||||**|||****|*||||||||||||||||||||****||*||*
SWVFGVHFCKQTYATYKMSYYSGMQQQQCTSIDRYVAIVQAVSAHRHRARTQQTSKQSCT
GIWILATVLSIPELLYSDLQRSSSEQAMRCSLITEHVEAFITIQVAQMVIGFLVPLLAMS
|*|**||**|*|||||||||||||||||||||||||||||||||||||**|***|**|||
GTWTQATTQSTPELLYSDLQRSSSEQAMRCSLITEHVEAFITIQVAQMTTGYQTPQQAMS
FCYLVIIRTLLQARNFERNKAIKVIIAVVVVFIVFQLPYNGVVLAQTVANFNITSSTCEL
*||****||**||||||||||||***|********|*||||***||||||||||||||||
YCYQTTTRTQQQARNFERNKAIKTTTATTTTYTTYQQPYNGTTQAQTVANFNITSSTCEL
SKQLNIAYDVTYSLACVRCCVNPFLYAFIGVKFRNDLFKLFKDLGCLSQEQLRQWSSCRH
|||||||||*|||*||*|||*||**||*||||||||||||||||||||||||||||||||
SKQLNIAYDTTYSQACTRCCTNPYQYAYIGVKFRNDLFKLFKDLGCLSQEQLRQWSSCRH
IRRSSMSVEAETTTTFSP
||||||||||||||||||
IRRSSMSVEAETTTTFSP

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native L, I V, and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 11: CCR-8 C-C Chemokine Receptor Type 8

Example 1 was repeated for the title protein. Replacing all or substantially all of the hydrophobic amino acids, L, I V, and F, with Q, T and Y (respectively) within the transmembrane domains results in the following sequence (lower line SEQ ID NO.:303), aligned with the wild type (top line SEQ ID NO. 302):

MDYTLDLSVTTVTDYYYPDIFSSPCDAELIQTNGKLLLAVFYCLLFVFSLLGNSLVILVL
|||||||||||||||||||||||||||||||||||||||**||*****|**|||******
MDYTLDLSVTTVTDYYYPDIFSSPCDAELIQTNGKLLLATYYCQQYTYSQQGNSQTTQTQ
VVCKKLRSITDVYLLNLALSDLLFVFSFPFQTYYLLDQWVFGTVMCKVVSGFYYIGFYSS
**|||||||||||**|*|*||*****|*|*||||**|||||||||||||||*||*|*|||
TTCKKLRSITDVYQQNQAQSDQQYTYSYPYQTYYQQDQWVFGTVMCKVVSGYYYTGYYSS
MFFITLMSVDRYLAVVHAVYALKVRTIRMGTTLCLAVWLTAIMATIPLLVFYQVASEDGV
|***|*||*|||||||||||||||||||||||||*|*|*||*|||*|****||*||||||
MYYTTQMSTDRYLAVVHAVYALKVRTIRMGTTLCQATWQTATMATTPQQTYYQTASEDGV
LQCYSFYNQQTLKWKIFTNFKMNILGLLIPFTIFMFCYIKILHQLKRCQNHNKTKAIRLV
|||||||||||||||**||*|||**|***|*|**|*||||||||||||||||||||||**
LQCYSFYNQQTLKWKTYTNYKMNTQGQQTPYTTYMYCYIKILHQLKRCQNHNKTKAIRQT
LIVVIASLLFWVPFNVVLFLTSLHSMHILDGCSISQQLTYATHVTEIISFTHCCVNPVIY
*****||***|*|*|*****||||||||||||||||||||||||||**|*||||*||**|
QTTTTASQQYWTPYNTTQYQTSLHSMHILDGCSISQQLTYATHVTETTSYTHCCTNPTTY
AFVGEKFKKHLSEIFQKSCSQIFNYLGRQMPRESCEKSSSCQQHSSRSSSVDYIL
|**||||||||||||||||||||||||||||||||||||||||||||||||||||
AYTGEKFKKHLSEIFQKSCSQIFNYLGRQMPRESCEKSSSCQQHSSRSSSVDYIL

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native L, I V, and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 12: CCR-9 C-C Chemokine Receptor Type 9 Isoform B

Example 1 was repeated for the title protein. Replacing all or substantially all of the hydrophobic amino acids, L, I V, and F, with Q, T and Y (respectively) within the transmembrane domains results in the following sequence (lower line SEQ ID NO: 305), aligned with the wild type (top line SEQ ID NO: 304):

MADDYGSESTSSMEDYVNFNFTDFYCEKNNVRQFASHFLPPLYWLVFIVGALGNSLVILV
|||||||||||||||||||||||||||||||||||||||||*||*****||*|||*****
MADDYGSESTSSMEDYVNFNFTDFYCEKNNVRQFASHFLPPQYWQTYTTGAQGNSQTTQT
YWYCTRVKTMTDMFLLNLAIADLLFLVTLPFWAIAAADQWKFQTFMCKVVNSMYKMNFYS
|||||||||||||***|*|*||*****|*|*||*|||||||||||||||||||||||*||
YWYCTRVKTMTDMYQQNQATADQQYQTTQPYWATAAADQWKFQTFMCKVVNSMYKMNYYS
CVLLIMCISVDRYIAIAQAMRAHTWREKRLLYSKMVCFTINVLAAALCIPEILYSQIKEE
|****||*|*|||*|*|||||||||||||**||||*|*|*|**|||*|*|||||||||||
CTQQTMCTSTDRYTATAQAMRAHTWREKRQQYSKMTCYTTWTQAAAQCTPEILYSQIKEE
SGIAICTMVYPSDESTKLKSAVLTLKVILGFFLPFVVMACCYTIIIHTLIQAKKSSKHKA
|||||||||||||||||||||||||||**|***|***||||||***||**||||||||||
SGIAICTMVYPSDESTKLKSAVLTLKVTQGYYQPYTTMACCYTTTTHTQTQAKKSSKHKA
LKVTITVLTVFVLSQFPYNCILLVQTIDAYAMFISNCAVSTNIDICFQVTQTIAFFHSCL
||*|*|**|****||*||||****||||||||||||||||||||||*|*|||*|**|||*
LKTTTTTQTTYTQSQYPYNCTQQTQTIDAYAMFISNCAVSTNIDICYQTTQTTAYYHSCQ
NPVLYVFVGERFRRDLVKTLKNLGCISQAQWVSFTRREGSLKLSSMLLETTSGALSL
||**|***|||||||||||||||||||||||||||||||||||||||||||||||||
NPTQYTYTGERFRRDLVKTLKNLGCISQAQWVSFTRREGSLKLSSMLLETTSGALSL

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native L, I V, and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 13: CCR-10 C-C Chemokine Receptor Type 10

Example 1 was repeated for the title protein. Replacing each of the hydrophobic amino acids, L, I V, and F, with Q, T and Y (respectively) within the transmembrane domains results in the following sequence (lower line SEQ ID NO: 307), aligned with the wild type (top line SEQ ID NO: 306):

MGTEATEQVSWGHYSGDEEDAYSAEPLPELCYKADVQAFSRAFQPSVSLTVAALGLAGNG
||||||||||||||||||||||||||||||||||||||||||||||*|*|*||*|*||||
MGTEATEQVSWGHYSGDEEDAYSAEPLPELCYKADVQAFSRAFQPSTSQTTAAQGQAGNG
LVLATHLAARRAARSPTSAHLLQLALADLLLALTLPFAAAGALQGWSLGSATCRTISGLY
***|||*|||||||||||||**|*|*||***|*|*|*|||||*|||||||||||||||*|
QTQATHQAARRAARSPTSAHQQQQAQADQQQAQTQPYAAAGAQQGWSLGSATCRTISGQY
SASFHAGFLFLACISADRYVAIARALPAGPRPSTPGRAHLVSVIVWLLSLLLALPALLFS
|||*|||****||*|||||||||||||||||||||||||**|***|**|***|*||***|
SASYHAGYQYQACTSADRYVAIARALPAGPRPSTPGRAHQTSTTTWQQSQQQAQPAQQYS
QDGQREGQRRCRLIFPEGLTQTVKGASAVAQVALGFALPLGVMVACYALLGRTLLAARGP
||||||||||||||||||||||||||||*||*|*|*|*|*|*|*||||**||||||||||
QDGQREGQRRCRLIFPEGLTQTVKGASATAQTAQGYAQPQGTMTACYAQQGRTLLAARGP
ERRRALRVVVALVAAFVVLQLPYSLALLLDTADLLAARERSCPASKRKDVALLVTSGLAL
|||||||***|**||****|*|||*|***||||||||||||||||||||*|***|||*|*
ERRRALRTTTAQTAAYTTQQQPYSQAQQQDTADLLAARERSCPASKRKDTAQQTTSGQAQ
ARCGLNPVLYAFLGLRFRQDLRRLLRGGSCPSGPQPRRGCPRRPRLSSCSAPTETHSLSW
||||*||**||**|||||||||||||||||||||||||||||||||||||||||||||||
ARCGQNPTQYAYQGLRFRQDLRRLLRGGSCPSGPQPRRGCPRRPRLSSCSAPTETHSLSW
DN
||
DN

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native L, I V and F amino acids, as set forth in the wild type sequence. The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 14: CXCR1 Chemokine Receptor Type 1

Example 1 was repeated for the title protein. Replacing all or substantially all of the hydrophobic amino acids, L, I V, and F, with Q, T and Y (respectively) within the transmembrane domains results in the following sequence (lower line SEQ ID NO: 309), aligned with the wild type (top line SEQ ID NO: 308):

MSNITDPQMWDFDDLNFTGMPPADEDYSPCMLETETLNKYVVIIAYALVFLLSLLGNSLV
||||||||||||||||||||||||||||||||||||||||****|||*****|**|||**
MSNITDPQMWDFDDLNFTGMPPADEDYSPCMLETETLNKYTTTTAYAQTYQQSQQGNSQT
MLVILYSRVGRSVTDVYLLNLALADLLFALTLPIWAASKVNGWIFGTFLCKVVSLLKEVN
|****||||||||||*|**|*|*||***|*|*|*||||||||||||||||||||||||||
MQTTQYSRVGRSVTDTYQQNQAQADQQYAQTQPTWAASKVNGWIFGTFLCKVVSLLKEVN
FYSGILLLACISVDRYLAIVHATRTLTQKRHLVKFVCLGCWGLSMNLSLPFFLFRQAYHP
*|||****||*|*|||*|**|||||||||||**|**|*||||*|||*|*|****||||||
YYSGTQQQACTSTDRYQATTHATRTLTQKRHQTKYTCQGCWGQSMNQSQPYYQYRQAYHP
NNSSPVCYEVLGNDTAKWRMVLRILPHTFGFIVPLFVMLFCYGFTLRTLFKAHMGQKHRA
||||||||||||||||||||||||||||*|***|***|**|||*|*||**||||||||||
NNSSPVCYEVLGNDTAKWRMVLRILPHTYGYTTPQYTMQYCYGYTQRTQYKAHMGQKHRA
MRVIFAVVLIFLLCWLPYNLVLLADTLMRTQVIQESCERRNNIGRALDATEILGFLHSCL
||***|*******||*|||***||||||||||||||||||||||||||||||*|**|||*
MRTTYATTQTYQQCWQPYNQTQLADTLMRTQVIQESCERRNNIGRALDATEIQGYQHSCQ
NPIIYAFIGQNFRHGFLKILAMHGLVSKEFLARHRVTSYTSSSVNVSSNL
||**||**||||||||||||||||||||||||||||||||||||||||||
NPTTYAYTGQNFRHGFLKILAMHGLVSKEFLARHRVTSYTSSSVNVSSNL

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native L, I V and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 15: CXR Chemokine Receptor 1 CXR1

Example 1 was repeated for the title protein. Replacing each of the hydrophobic amino acids, L, I V, and F, with Q, T and Y (respectively) within the transmembrane domains results in the following sequence (lower line SEQ ID NO: 311), aligned with the wild type (top line SEQ ID NO: 310):

MESSGNPESTTFFYYDLQSQPCENQAWVFATLATTVLYCLVFLLSLVGNSLVLWVLVKYE
|||||||||||||||||||||||||||||||||||**||*****|**|||***|**||||
MESSGNPESTTFFYYDLQSQPCENQAWVFATLATTTQYCQTYQQSQTGNSQTQWTQVKYE
SLESLTNIFILNLCLSDLVFACLLPVWISPYHWGWVLGDFLCKLLNMIFSISLYSSIFFL
|||||||****|*|*||***||**|*|*||||||||||||||||||||||*|*|||****
SLESLTNTYTQNQCQSDQTYACQQPTWTSPYHWGWVLGDFLCKLLNMIFSTSQYSSTYYQ
TIMTIHRYLSVVSPLSTLRVPTLRCRVLVTMAVWVASILSSILDTIFHKVLSSGCDYSEL
|*||*|||*|**||||||||||||||***|||*|*||**||**||**|||||||||||||
TTMTTHRYQSTTSPLSTLRVPTLRCRTQTTMATWTASTQSSTQDTTYHKVLSSGCDYSEL
TWYLTSVYQHNLFFLLSLGIILFCYVEILRTLFRSRSKRRHRTVKLIFAIVVAYFLSWGP
||||||*||||*****|*|****||*|**||||||||||||||||***|***||**||||
TWYLTSTYQHNQYYQQSQGTTQYCYTETQRTLFRSRSKRRHRTVKQTYATTTAYYQSWGP
YNFTLFLQTLFRTQIIRSCEAKQQLEYALLICRNLAFSHCCFNPVLYVFVGVKFRTHLKH
||*|***|||||||||||||||||||||***|||*|*||||*||**|***||||||||||
YNYTQYQQTLFRTQIIRSCEAKQQLEYAQQTCRNQAYSHCCYNPTQYTYTGVKFRTHLKH
VLRQFWFCRLQAPSPASIPHSPGAFAYEGASFY
|||||||||||||||||||||||||||||||||
VLRQFWFCRLQAPSPASIPHSPGAFAYEGASFY

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native V, L I and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 16: CXCR2 Chemokine Receptor Type 2

Example 1 was repeated for the title protein. Replacing all or substantially all of the hydrophobic amino acids, L, I V, and F, with Q, T and Y (respectively) within the transmembrane domains results in the following sequence (lower line SEQ ID NO: 313), aligned with the wild type (top line SEQ ID NO: 312):

MEDFNMESDSFEDFWKGEDLSNYSYSSTLPPFLLDAAPCEPESLEINKYFVVIIYALVFL
||||||||||||||||||||||||||||||||||||||||||||||||||****||****
MEDFNMESDSFEDFWKGEDLSNYSYSSTLPPFLLDAAPCEPESLEINKYFTTTTYAQTYQ
LSLLGNSLVMLVILYSRVGRSVTDVYLLNLALADLLFALTLPIWAASKVNGWIFGTFLCK
*|**|||**|***|||||||||||*|**|*|*||***|*|*|*|||||||||||||||||
QSQQGNSQTMQTTLYSRVGRSVTDTYQQNQAQADQQYAQTQPTWAASKVNGWIFGTFLCK
VVSLLKEVNFYSGILLLACISVDRYLAIVHATRTLTQKRYLVKFICLSIWGLSLLLALPV
|||||||*|*|||****||*|*|||*|**|||||||||||**|**|*|*||*|***|*|*
VVSLLKETNYYSGTQQQACTSTDRYQATTHATRTLTQKRYQTKYTCQSTWGQSQQQAQPT
LLFRRTVYSSNVSPACYEDMGNNTANWRMLLRILPQSFGFIVPLLIMLFCYGFTLRTLFK
***||||||||||||||||||||||||||||||||||*|***|***|**|||*|*||**|
QQYRRTVYSSNVSPACYEDMGNNTANWRMLLRILPQSYGYTTPQQTMQYCYGYTQRTQYK
AHMGQKHRAMRVIFAVVLIFLLCWLPYNLVLLADTLMRTQVIQETCERRNHIDRALDATE
|||||||||||***|*******||*|||***|||||||||||||||||||||||||||||
AHMGQKHRAMRTTYATTQTYQQCWQPYNQTQLADTLMRTQVIQETCERRNHIDRALDATE
ILGILHSCLNPLIYAFIGQKFRHGLLKILAIHGLISKDSLPKDSRPSFVGSSSGHTSTTL
**|**|||*||**||**|||||||||||||||||||||||||||||||||||||||||||
TQGTQHSCQNPQTYAYTGQKFRHGLLKILAIHGLISKDSLPKDSRPSFVGSSSGHTSTTL

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native L, I V and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 17: CCR-10 C-C Chemokine Receptor Type 10

Example 1 was repeated for the title protein. Replacing each of the hydrophobic amino acids, L, I V, and F, with Q, T and Y (respectively) within the transmembrane domains results in the following sequence (lower line SEQ ID NO: 315), aligned with the wild type (top line SEQ ID NO: 314):

MNYPLTLEMDLENLEDLFWELDRLDNYNDTSLVENHLCPATEGPLMASFKAVFVPVAYSL
|||||||||||||||||||||||||||||||||||||||||||||||||||||*|*|||*
MNYPLTLEMDLENLEDLFWELDRLDNYNDTSLVENHLCPATEGPLMASFKAVFTPTAYSQ
IFLLGVIGNVLVLVILERHRQTRSSTETFLFHLAVADLLLVFILPFAVAEGSVGWVLGTF
****|**||*******||||||||||||***|*|*||*******|*|*||||||||||||
TYQQGTTGNTQTQTTQERHRQTRSSTETYQYHQATADQQQTYTQPYATAEGSVGWVLGTF
LCKTVIALHKVNFYCSSLLLACIAVDRYLAIVHAVHAYRHRRLLSIHITCGTIWLVGFLL
|||||*|*||*|*||||***||*|*||||||||||||||||||||*|*||||*|**|***
LCKTVTAQHKTNYYCSSQQQACTATDRYLAIVHAVHAYRHRRLLSTHTTCGTTWQTGYQQ
ALPEILFAKVSQGHHNNSLPRCTFSQENQAETHAWFTSRFLYHVAGFLLPMLVMGWCYVG
|*||***||||||||||||||||||||||||||||||||**||*||***||**|||||*|
AQPETQYAKVSQGHHNNSLPRCTFSQENQAETHAWFTSRYQYHTAGYQQPMQTMGWCYTG
VVHRLRQAQRRPQRQKAVRVAILVTSIFFLCWSPYHIVIFLDTLARLKAVDNTCKLNGSL
**|||||||||||||||*|*|***||****||||||****|||||||||||||||||||*
TTHRLRQAQRRPQRQKATRTATQTTSTYYQCWSPYHTTTYLDTLARLKAVDNTCKLNGSQ
PVAITMCEFLGLAHCCLNPMLYTFAGVKFRSDLSRLLTKLGCTGPASLCQLFPSWRRSSL
|*|*||||**|*||||*|||*|||||||||||||||||||||||||||||||||||||||
PTATTMCEYQGQAHCCQNPMQYTFAGVKFRSDLSRLLTKLGCTGPASLCQLFPSWRRSSL
SESENATSLTTF
||||||||||||
SESENATSLTTF

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native L, I V and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 18: CXCR6 Chemokine Receptor Type 6

Example 1 was repeated for the title protein. Replacing each of the hydrophobic amino acids, L, I V, and F, with Q, T and Y (respectively) within the transmembrane domains results in the following sequence (lower line SEQ ID NO: 317), aligned with the wild type (top line SEQ ID NO: 316):

MAEHDYHEDYGFSSFNDSSQEEHQDFLQFSKVFLPCMYLVVFVCGLVGNSLVLVISIFYH
||||||||||||||||||||||||||||||||||||||*****||**|||*****|**||
MAEHDYHEDYGFSSFNDSSQEEHQDFLQFSKVFLPCMYQTTYTCGQTGNSQTQTTSTYYH
KLQSLTDVFLVNLPLADLVFVCTLPFWAYAGTHEWVFGQVMCKSLLGIYTINFYTSMLIL
|||||||****|*|*||****||*|*||||||||||||||||||||||||*|*||||***
KLQSLTDTYQTNQPQADQTYTCTQPYWAYAGIHEWVFGQVMCKSLLGIYTTNYYTSMQTQ
TCITVDRFIVVVKATKAYNQQAKRMTWGKVTSLLIWVISLLVSLPQIIYGNVFNLDKLIC
||*|*||*****||||||||||||||||||||***|**|***|*||**|||**|*|||||
TCTTTDRYTTTTKATKAYNQQAKRMTWGKVTSQQTWTTSQQTSQPQTTYGNTYNQDKLIC
GYHDEAISTVVLATQMTLGFFLPLLTMIVCYSVIIKTLLHAGGFQKHRSLKIIFLVMAVF
|||||||||***|||||*|***|**||**||||||||||||||||||||||*****||**
GYHDEAISTTTQATQMTQGYYQPQQTMTTCYSVIIKTLLHAGGFQKHRSLKTTYQTMATY
LLTQMPFNLMKFIRSTHWEYYAMTSFHYTIMVTEAIAYLRACLNPVLYAFVSLKFRKNFW
**||||*|*||**||||||||||||||||*|*|||*||*|||*||**||**|||||||||
QQTQMPYNQMKYTRSTHWEYYAMTSFHYTTMTTEATAYQRACQNPTQYAYTSLKFRKNFW
KLVKDIGCLPYLGVSHQWKSSEDNSKTFSASHNVEATSMFQL
||||||||||||||||||||||||||||||||||||||||||
KLVKDIGCLPYLGVSHQWKSSEDNSKTFSASHNVEATSMFQL

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native L, I V and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 19: CXCR7 Chemokine Receptor Type 7

Example 1 was repeated for the title protein. Replacing all or substantially all of the hydrophobic amino acids, L, I V, and F, with Q, T and Y (respectively) within the transmembrane domains results in the following sequence (lower line SEQ ID NO: 319), aligned with the wild type (top line SEQ ID NO: 318):

MDLHLFDYSEPGNFSDISWPCNSSDCIVVDTVMCPNMPNKSVLLYTLSFIYIFIFVIGMI
||||||||||||||||||||||||||||||||||||||||||||||*|**|******||*
MDLHLFDYSEPGNFSDISWPCNSSDCIVVDTVMCPNMPNKSVLLYTQSYTYTYTYTTGMT
ANSVVVWVNIQAKTTGYDTHCYILNLAIADLWVVLTIPVWVVSLVQHNQWPMGELTCKVT
|||***|*||||||||||||||**|*|*||*|***|*|*|**|*||||||||||||||*|
ANSTTTWTNIQAKTTGYDTHCYTQNQATADQWTTQTTPTWTTSQVQHNQWPMGELTCKTT
HLIFSINLFGSIFFLTCMSVDRYLSITYFTNTPSSRKKMVRRVVCILVWLLAFCVSLPDT
|***|*|**||****||||*|||||||||||||||||||*||**|***|**|*|*|*|||
HQTYSTNQYGSTYYQTCMSTDRYLSITYFTNTPSSRKKMTRRTTCTQTWQQAYCTSQPDT
YYLKTVTSASNNETYCRSFYPEHSIKEWLIGMELVSVVLGFAVPFSIIAVFYFLLARAIS
|||||||||||||||||||||||||||||||||**|***|*|*|*|**|**|***|||||
YYLKTVTSASNNETYCRSFYPEHSIKEWLIGMEQTSTTQGYATPYSTTATYYYQQARAIS
ASSDQEKHSSRKIIFSYVVVFLVCWLPYHVAVLLDIFSILHYIPFTCRLEHALFTALHVT
||||||||||||||*||******||*|||*|***|**|||||||||||||||||||*|*|
ASSDQEKHSSRKIIYSYTTTYQTCWQPYHTATQQDTYSILHYIPFTCRLEHALFTAQHTT
QCLSLVHCCVNPVLYSFINRNYRYELMKAFIFKYSAKTGLTKLIDASRVSETEYSALEQS
||*|**|||*||**||**||||||||||||||||||||||||||||||||||||||||||
QCQSQTHCCTNPTQYSYTNRNYRYELMKAFIFKYSAKTGLTKLIDASRVSETEYSALEQS
TK
||
TK

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native L, I V and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 20: CLR-1a Chemokine Like Receptor 1 Isoform a

Example 1 was repeated for the title protein. Replacing all or substantially all of the hydrophobic amino acids, L, I V, and F, with Q, T and Y (respectively) within the transmembrane domains results in the following sequence (lower line SEQ ID NO: 321), aligned with the wild type (top line SEQ ID NO: 320):

MRMEDEDYNTSISYGDEYPDYLDSIVVLEDLSPLEARVTRIFLVVVYSIVCFLGILGNGL
||||||||||||||||||||||||||||||||||||||||******||**|**|**|||*
MRMEDEDYNTSISYGDEYPDYLDSIVVLEDLSPLEARVTRTYQTTTYSTTCYQGTQGNGQ
VIIIATFKMKKTVNMVWFLNLAVADFLFNVFLPTHITYAAMDYHWVFGTAMCKISNFLLI
***||||||||||||*|**|*|*||***|***|*|*|||||||||||||||||||||***
TTTIATFKMKKTVNMTWYQNQATADYQYNTYQPTHTTYAAMDYHWVFGTAMCKISNFQQT
HNMFTSVFLLTIISSDRCISVLLPVWSQNHRSVRLAYMACMVIWVLAFFLSSPSLVFRDT
|||*||****|**|||||||||||||||||||||*||||||**|**|***||||***|||
HNMYTSTYQQTTTSSDRCISVLLPVWSQNHRSVRQAYMACMTTWTQAYYQSSPSQTYRDT
ANLHGKISCFNNFSLSTPGSSSNPTHSQMDPVGYSRHMVVTVTRFLCGFLVPVLIITACY
||||||||||||||||||||||||||||||||||||||||||||**||***|****||||
ANLHGKISCFNNFSLSTPGSSSWPTHSQMDPVGYSRHMVVTVTRYQCGYQTPTQTTTACY
LTIVCKLQRNRLAKTKKPFKIIVTIIITFFLCWCPYHTLNLLELHHTAMPGSVFSLGLPL
*|**||*|||||||||||*|***|***|***|||||||*|*||||||||||||||*|*|*
QTTTCKQQRNRLAKTKKPYKTTTTTTTTYYQCWCPYHTQNQLELHHTAMPGSVFSQGQPQ
ATALAIANSCMNPILYVFMGQDFKKFKVALFSRLVNALSEDTGHSSYPSHRSFTKMSSMN
|||*|*|||||||**|**||||||||||||||||||||||||||||||||||||||||||
ATAQATANSCMNPTQYTYMGQDFKKFKVALFSRLVNALSEDTGHSSYPSHRSFTKMSSMN
ERTSMNERETGML
|||||||||||||
ERTSMNERETGML

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native L, I V and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 21: DARIA Duffy Antigen/Chemokine Receptor Isoform a

Example 1 was repeated for the title protein. Replacing each of the hydrophobic amino acids, L, I V, and F, with Q, T and Y (respectively) within the transmembrane domains results in the following sequence (lower line SEQ ID NO: 323), aligned with the wild type (top line SEQ ID NO: 322):

MASSGYVLQAELSPSTENSSQLDFEDVWNSSYGVNDSFPDGDYGANLEAAAPCHSCNLLD
||||||||||||||||||||||||||||||||||||||||||||||||||||||||||||
MASSGYVLQAELSPSTENSSQLDFEDVWNSSYGVNDSFPDGDYGANLEAAAPCHSCNLLD
DSALPFFILTSVLGILASSTVLFMLFRPLFRWQLCPGWPVLAQLAVGSALFSIVVPVLAP
|||*|****||**|**||||***|*||||||||||||||**||*|*|||**|***|**||
DSAQPYYTQTSTQGTQASSTTQYMQFRPLFRWQLCPGWPTQAQQATGSAQYSTTTPTQAP
GLGSTRSSALCSLGYCVWYGSAFAQALLLGCHASLGHRLGAGQVPGLTLGLTVGIWGVAA
||||||||||||||||*|||||*|||***|||||*|||||||||||||*|*|*|*||*||
GLGSTRSSALCSLGYCTWYGSAYAQAQQQGCHASQGHRLGAGQVPGLTQGQTTGTWGTAA
LLTLPVTLASGASGGLCTLIYSTELKALQATHTVACLAIFVLLPLGLFGAKGLKKALGMG
**|*|*|*|||||||||||||||||||||||||*||*|*****|*|**||||*|||||||
QQTQPTTQASGASGGLCTLIYSTELKALQATHTTACQATYTQQPQGQYGAKGQKKALGMG
PGPWMNILWAWFIFWWPHGVVLGLDFLVRSKLLLLSTCLAQQALDLLLNLAEALAILHCV
||||||**|||***|||||***|*|***|||||||||||||||||||*|*|||*|**||*
PGPWMNTQWAWYTYWWPHGTTQGQDYQTRSKLLLLSTCLAQQALDLLQNQAEAQATQHCT
ATPLLLALFCHQATRTLLPSLPLPEGWSSHLDTLGSKS
|||***|**|||||||||||||||||||||||||||||
ATPQQQAQYCHQATRTLLPSLPLPEGWSSHLDTLGSKS

Each of the predicted transmembrane regions has been underlined and exemplified a fully modified domain of the invention. Thus, for example, the invention includes a transmembrane domain comprising each underlined domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native L, I, V and F amino acids, as set forth in the wild type sequence. The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 22: CD81 Antigen

CD81 may play an important role in the regulation of lymphoma cell growth and interacts with a 16 kDa Leu-13 protein to form a complex possibly involved in signal transduction. CD81 may act as a viral receptor for HCV.

Example 1 was repeated for the title protein. Replacing each of the hydrophobic amino acids, L, I V, and F, with Q, T and Y (respectively) within the transmembrane domains results in the following sequence (lower line SEQ ID NO: 325), aligned with the wild type (top line SEQ ID NO: 324):

| | | |
|---|---|---|
| WT: | 1 | MGVEGCTKCIKYLLFVFNFVFWLAGGVILGVALWLRHDPQTTNLLYLELGDKPAPNTFYV |
| | | \|\|\|\|\|\|\|\|\|\|\|\|*****\|***\|*\|\|\|***\|*\|*\|\|\|\|\|\|\|\|\|\|\|\|\|\|\|\|\|\|\|\|\|\|\|\|\|\|\| |
| MT: | 1 | MGVEGCTKCIKYQQYTYNYTYWQAGGTTQGTAQWLRHDPQTTNLLYLELGDKPAPNTFYV |
| WT: | 61 | GIYILIAVGAVMMFVGFLGCYGAIQESQCLLGTFFTCLVILFACEVAAGIWGFVNKDQIA |
| | | \|\|\|***\|*\|\|*\|\|**\|**\|\|\|\|\|*\|\|\|\|\|**\|\|**\|\|*****\|\|\|*\|\|\|*\|\|\|\|\|\|\|\|\|\| |
| MT: | 61 | GIYTQTATGATMMYTGYQGCYGATQESQCQQGTYYTCQTTQACETAAGTWGFVNKDQIA |
| WT: | 121 | KDVKQFYDQALQQAVVDDDANNAKAVVKTFHETLDCCGSSTLTALTTSVLKNNLCPSGSN |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| MT: | 121 | KDVKQFYDQALQQAVVDDDANNAKAVVKTFHETLDCCGSSTLTALTTSVLKNNLCPSGSN |
| WT: | 181 | IISNLFKEDCHQKIDDLFSGKLYLIGIAAIVVAVIMIFEMILSMVLCCGIRNSSVY |
| | | \|\|\|\|\|\|\|\|\|\|\|\|\|\|\|\|\|\|\|\|\|*\|**\|*\|\|***\|**\|**\|\|**\|\|\|\|\|\|\|\|\|\|\|\|\|\| |
| MT: | 181 | IISNLFKEDCHQKIDDLFSGKQYQTGTAATTTATTMTYEMTQSMVLCCGIRNSSVY |

The predicted transmembrane regions exemplify modified domains of the invention and include (SEQ ID NOs: 326, 327, 328, 329, 330, 331, 332, 333, respectively):

| | |
|---|---|
| TM1-wt: | LFVFNFVFWLAGGVILGVALW |
| | ****\|***\|*\|\|\|***\|*\|*\| |
| TM1-mt: | QYTYNYTYWQAGGTTQGTAQW |
| TM2-wt: | LIAVGAVMMFVGFLGCYGAIQ |
| | **\|*\|\|*\|\|**\|**\|\|\|\|\|*\| |
| TM2-mt: | QTATGATMMYTGYQGCYGATQ |
| TM3-wt: | LGTFFTCLVILFACEVAAGIWGF |
| | *\|\|**\|\|*****\|\|\|*\|\|\|*\|\|\| |
| TM3-mt: | QGTYYTCQTTQYACETAAGTWGF |
| TM4-wt: | YLIGIAAIVVAVIMIFEMILSMV |
| | \|**\|*\|\|***\|**\|**\|\|**\|\|\| |
| TM4-mt: | YQTGTAATTTATTMTYEMTQSMV |

Thus, for example, the invention includes a transmembrane domain comprising each modified or “mt” domain. Preferably the protein comprising TM1 herein includes one or more (e.g., all) of the extracellular and intracellular loop sequences (the sequences which have not been underlined). In addition or alternatively, the protein comprising the TM1 herein includes one or more additional transmembrane regions (the underlined sequences) in the depicted protein or homologous sequences retaining one, two, three or, possibly four or more of the native V, L I and F amino acids, as set forth in the wild type sequence.

The wild type sequence can be subject to the process as discussed above to select additional transmembrane domain variants as described in Example 1. Coding sequences can be designed, shuffled and proteins expressed. The expressed proteins can be assayed for ligand binding, as described herein.

### Example 23: E. coli Expression of QTY Variants and E. coli Expression of a CXCR4-QTY Variant

1. Large-Scale Production of CXCR4-QTY in E. coli BL21 (DE3)

A water-soluble GPCR CXCR4 was produced it in E. coli with a yield estimated to be ˜20 mg purified protein per liter of routine LB culture media. The estimated cost of production is about $0.25 per milligram. Advantageously, this approach can be used to easily obtain grams of quantity of water-soluble GPCRs, which in turn can facilitate their structural determinations.

2. Determining where the Water-Soluble CXCR4-QTY is Produced in E. coli Cells

A water-soluble CXCR4-QTY was cloned into pET vector. We first carried out a small-scale E. coli culture study to assess the location of produced CXCR4-QTY protein (150 ml culture). After culturing the cells, induced with IPTG at 24° C. for 4 hours, we collected and sonicated the cells and divided into 2 fractions through centrifugation at 14,637×g (12,000 rmp). We then used Western blot analysis of the specific anti-rho-tag monoclonal antibody to detect where the CXCR4-QTY protein was. We observed that the CXCR4-QTY protein was in the supernatant fraction and no protein was observed in the pellet fraction, thus suggesting the protein is fully water-soluble.

3. The Estimated Yield CXCR4-QTY Produced in Soluble Fraction of E. coli Cells

We then carried out another 150 ml culture and obtained ˜6 mg 1D4 monoclonal antibody-purified CXCR4-QTY. Because we under-estimated the yield (we did not anticipate the surprisingly high yield), we did not use enough affinity 1D4 rho-tag monoclonal antibody beads to capture the produced CXCR4-QTY. Thus, a significant amount CXCR4-QTY protein did not bind to the beads due to the fact that not enough beads were added during purification, and the protein was in the flow-through lane and was further washed out. Despite the significant loss, we are still able to obtain ˜6 mg for the 150 ml culture as seen from the lanes 8-10 (Elution fractions).

### 4. Measuring the Thermo-Stability of Purified Water-Soluble CXCR4-QTY Protein

In most cases, structure determines function in proteins. Thus it is important to know if the purified CXCR4-QTY protein produced in E. coli still folds correctly in the typical alpha-helical structure with ˜50% alpha-helix. We performed secondary structural measurement using Circular dichroism (CD). We observed the CD spectra of purified CXCR4-QTY protein at various temperatures. We measured the thermo-stability of purified CXCR4-QTY protein. We observed that the purified CXCR4-QTY protein is relatively stable up to 55° C., the protein was only partially and gradually denatured, the CD signal reduction was ˜15%. Between 55° C. and 65° C., the denaturation increased toward 65° C., the denaturation transition took place between 65° C. and 75° C. and the protein was nearly fully denatured at 75° C.

We plotted the temperature vs the ellipticity at 222 nm to obtain the melting temperature (Tm) of purified water-soluble CXCR4-QTY protein. From the plot, we estimated that the Tm for purified CXCR4-QTY protein is ˜67° C. This Tm suggests the purified water-soluble CXCR4-QTY protein is quite stable compared to many other soluble proteins. This thermo-stability characteristics facilitates obtaining diffracting crystals, since it is known that the better the thermo-stability, the better the crystal lattice packing, and thus the better the chances to obtain structures.

### 5. Additional G Protein-Coupled Receptors

We selected 10 G protein-coupled receptors (GPCRs) to design the water-soluble form, using the QTY method that is described in Zhang et al., Water Soluble Membrane Proteins and Methods for the Preparation and Use Thereof, U.S. Patent Publication No. 2012/0252719 A (“Zhang”). Alternatively, the proteins described herein can be selected.

### 6. Molecular Cloning of the Genes.

We successfully verified the GPCR native and QTY genes in the cell-free protein expression plasmid vector pIVex2.3d and E. coli pET28a and pET-duet-1 plasmid vectors.

### 7. Water-Soluble GPCR Productions

We have produced several native and QTY proteins. When producing native GPCR in the cell-free system, a detergent Brij35 is required, without the detergent, the proteins precipitate upon production. On the other hand, we tested QTY variants in the present and absent of detergent. Without the detergent, the cell-free system produced soluble proteins.

We cloned the QTY variants into E. coli in vivo expression system, pET28a and pET-duet-1 plasmid vectors for E. coli cell protein production in E. coli BL21 (DE3) strain. We have purified several water-soluble GPCR proteins, including CXCR4 and CCR5, which we have used for secondary structural analysis. We have performed ligand-binding studies for CXCR4 with its natural ligand CCL12 (SDF1a). We carried out E. coli production and purification of water-soluble GPCR CCR5e variant. The CCR5e variant had 58 amino acid changes (˜18% change). The water-soluble GPCR CCR5e variant was purified to homogeneity using the specific monoclonal antibody rhodopsin-tag. The blue stain showed a single band on the SDS gel indicating the purity. Estimated from the protein size marker, it appears to be a pure homo-dimer (the native membrane-bound CXCR4 crystal structure was a dimer. The Western-blot verified the monomer and homo-dimmer of CCR5e variant that is common in GPCRs.

### 8. QTY CCR5e Secondary Structural Studies.

We obtained water-soluble QTY variant of GPCR CCR5e. Then we carried out secondary structural analyses using an Aviv Model 410 circular dichroism instrument and confirmed that the GPCR QTY CCR5-e variant has a typical alpha-helical structure. We also carried out experiments in various temperatures to determine the CCR5e variant Tm, namely, the thermo-stability of the water-soluble CCR5e variant. From the experiments, we determined the Tm of CCR5e variant is about 46° C. This Tm is good for crystal screen experiments.

9. Ligand-Binding Studies of CXCR4 with CCL12 (SDF1a).

In order to be certain the designed water-soluble QTY GPCRs still maintain their biological function, namely recognize and bind to their natural ligands, we first used an ELISA measurement to study water-soluble CXCR4 with its natural ligand CCL12 (also called SDF1a). The assay concentrations range from 50 nM to 10 μM. The measured Kd is ˜80 nM. The Kd of native membrane-bound CXCR4 with SDF1a is about 100 nM. So the Kd of water-soluble CXCR4 is within acceptable range. Further experiments using more sensitive SPR or other measurement may produce more accurate Kd.

While this invention has been particularly shown and described with references to preferred embodiments thereof, it will be understood by those skilled in the art that various changes in form and details may be made therein without departing from the scope of the invention encompassed by the appended claims.

1. A process of designing a water-soluble transmembrane protein, wherein the transmembrane protein is a G Protein-Coupled Receptor (GPCR), the method comprising:
(1) generating a first library of putative water-soluble first modified transmembrane variants of a first transmembrane region of a native transmembrane protein, wherein each of said variants is generated by replacing a plurality of hydrophobic amino acids of the first transmembrane region, wherein the plurality of hydrophobic amino acids are selected from the group consisting of: Leucine (L), isoleucine (I), valine (V), and phenylalanine (F), and wherein each leucine, isoleucine, valine, and phenylalanine is replaced with a non-ionic polar amino acid selected from the group consisting of Q (or alternatively, N or S), T (or alternatively, N or S), T (or alternatively, N or S) and Y, respectively;
(2) comparing structure scores and solubility scores of each said putative water-soluble first modified transmembrane variants in the first library and, preferably ranking the putative water-soluble first modified transmembrane variants using said structure scores and solubility scores in order to arrive at a second library of putative water-soluble first modified transmembrane variants, wherein said structure scores are obtained by scoring the propensity of forming alpha-helical structure of the first transmembrane regions of said variants, and wherein said solubility scores are obtained by scoring the water solubility prediction of the first transmembrane regions of said variants;
(3) repeating steps (1) through (2) for a second, third, fourth, fifth, sixth, seventh or, preferably, all transmembrane regions of the protein;
(4) identifying the amino acid and/or nucleic acid sequences of the water-soluble transmembrane protein comprising sequences which are not included in any transmembrane regions modified in steps (1) through (3), and including any extracellular or intracellular domain of the water-soluble transmembrane protein, thereby designing said water-soluble transmembrane protein.

2. (canceled)

3. The method of claim 1, wherein the number of the putative water-soluble first modified transmembrane variants is an integer, H, selected from the group consisting of 10, 9, 8, 7, 6, 5, 4, 3, 2, and 1.

4. The method of claim 3, wherein the sum of the transmembrane regions modified by the method is an integer, n, selected from the group consisting of 7, 6, 5, 4, 3, 2, and 1.

5. The method of claim 1, wherein all or substantially all of the leucines (L) in the transmembrane domains are replaced with glutamines (Q); wherein all or substantially all of the valines (V) in the transmembrane domains are replaced with threonines (T); wherein all or substantially all of the isoleucines (I) in the transmembrane domains are replaced with threonines (T); or wherein all or substantially all of the phenylalanines (F) in the transmembrane domains are replaced with tyrosines (Y).

6-8. (canceled)

9. The method of claim 5, wherein all or substantially all of the L, V, I and/or Fs in the transmembrane domains are replaced with Q, T, T and Y, respectively.

10. The method of claim 1, wherein one or more isoleucines in the transmembrane domain are not replaced, or wherein one or more phenylalanines in the transmembrane domain are not replaced.

11. (canceled)

12. The method of claim 1, wherein the one or more transmembrane domains selected in step (2) or (3) contain 0, 1, 2 or 3 hydrophobic amino acids selected from the group consisting of L, V, I and F.

13. The method of claim 1, wherein the water solubility score is selected based on the prediction of the required water solubility of the conditions of use for the water-soluble transmembrane protein.

14. The method of claim 13 wherein the conditions of use for the water-soluble transmembrane protein is an aqueous ligand binding assay.

15. The method of claim 13 wherein a transmembrane domain that receives a water solubility score less than the prediction of the required water solubility of the conditions of use is discarded.

16. The method of claim 1, further comprising the steps:
a. Expressing a plurality of water-soluble transmembrane proteins encoded by the nucleic acid sequences of claim 1; and
b. Screening at least a portion of the plurality of water-soluble transmembrane proteins produced in step a for ligand binding.

17. The method of claim 16, further comprising the step of sequencing at least one water soluble transmembrane protein that binds ligand in step b.

18. The method of claim 17, further comprising the step of assaying the ligand selectivity of the water-soluble transmembrane protein.

19. The method of claim 4, wherein Hⁿ is less than about 2,000,000.

20. The method of claim 16, wherein the number of the plurality of water-soluble transmembrane proteins screened is less than about 1% of 2,000,000.

21-73. (canceled)

74. The method of claim 1, which is a computer implemented method performed on a computer system programmed to carry out the steps of claim 1.

75. (canceled)

76. A non-transient computer readable medium having computer-executable instructions stored thereon, the computer-executable instructions, when executed by a computer system, causing the computer system to perform the steps of claim 1.

77. A computer system comprising:
a. a memory;
b. at least one processor connected to the memory, the processor being configured to perform the steps of claim 1, such that the memory stores modified transmembrane variants and structured scores.


SEQUENCE LISTING (382 sequences)

SEQ ID NO: 1 - protein, 356 aa, Unknown
  Description of Unknown Mammalian CXCR4 polypeptide
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 2 - protein, 356 aa, Artificial Sequence
  Description of Artificial Sequence Synthetic polypeptide
MSIPLPLLQIYTSDNYTEEMGSGDYDSMKEPCFREENANFNKIFLPTTYSTTYQTGTTGNGQTTQTMGYQKKLRSMTDKYRQHQSTADQQYTTTQPYWATDAVANWYFGNFLCKATHTTYTTNQYSSTQTQAYTSQDRYLAIVHATNSQRPRKLLAEKTTYTGTWTPAQQQTTPDYTYANVSEADDRYICDRFYPNDLWVVVYQYQHTMTGQTQPGTTTQSCYCTIISKLSHSKGHQKRKALKTTTTQTQAYYACWQPYYTGTSTDSYILLEIIKQGCEFENTVHKWTSTTEAQAYYHCCQNPTQYAYQGAKFKTSAQHALTSVSRGSSLKILSKGKRGGHSSVSTESESSSFHSS

SEQ ID NO: 3 - protein, 38 aa, Unknown
  Description of Unknown Mammalian CXCR4 polypeptide
MEGISIYTSDNYTEEMGSGDYDSMKEPCFREENANFNK

SEQ ID NO: 4 - protein, 25 aa, Artificial Sequence
  Description of Artificial Sequence Synthetic peptide
IFLPTTYSTTFQTGTTGNGQVTQVM

SEQ ID NO: 5 - protein, 25 aa, Artificial Sequence
  Description of Artificial Sequence Synthetic peptide
IFQPTTYSTTFQTGTTGNGQVTQVM

SEQ ID NO: 6 - protein, 25 aa, Artificial Sequence
  Description of Artificial Sequence Synthetic peptide
IFQPTTYSTTFQTGTTGNGQVTQTM

SEQ ID NO: 7 - protein, 25 aa, Artificial Sequence
  Description of Artificial Sequence Synthetic peptide
IFQPTTYSTTYQTGTTGNGQVTQTM

SEQ ID NO: 8 - protein, 25 aa, Artificial Sequence
  Description of Artificial Sequence Synthetic peptide
IFQPTTYSTTYQTGTTGNGQTTQVM

SEQ ID NO: 9 - protein, 25 aa, Artificial Sequence
  Description of Artificial Sequence Synthetic peptide
IFQPTTYSTTYQTGTTGNGQTIQTM

SEQ ID NO: 10 - protein, 25 aa, Artificial Sequence
  Description of Artificial Sequence Synthetic peptide
IFQPTTYSTTYQTGTTGNGQTTQTM

SEQ ID NO: 11 - protein, 25 aa, Artificial Sequence
  Description of Artificial Sequence Synthetic peptide
TYQPTTYSTTYQTGTTGNGQTTQTM

SEQ ID NO: 12 - protein, 14 aa, Unknown
  Description of Unknown Mammalian CXCR4 peptide
GYQKKLRSMTDKYR

SEQ ID NO: 13 - protein, 22 aa, Artificial Sequence
  Description of Artificial Sequence Synthetic peptide
LHLSTADQQFTTTQPFWAVDAV

SEQ ID NO: 14 - protein, 22 aa, Artificial Sequence
  Description of Artificial Sequence Synthetic peptide
LHLSVADQQYTTTQPFWATDAV

SEQ ID NO: 15 - protein, 22 aa, Artificial Sequence
  Description of Artificial Sequence Synthetic peptide
LHQSVADQQYVTTQPFWATDAT

SEQ ID NO: 16 - protein, 22 aa, Artificial Sequence
  Description of Artificial Sequence Synthetic peptide
QHQSVADQQFTTTQPFWATDAT

SEQ ID NO: 17 - protein, 22 aa, Artificial Sequence
  Description of Artificial Sequence Synthetic peptide
LHQSVADQQYTITQPYWATDAT

SEQ ID NO: 18 - protein, 22 aa, Artificial Sequence
  Description of Artificial Sequence Synthetic peptide
QHLSVADQQYTITQPYWATDAT

SEQ ID NO: 19 - protein, 22 aa, Artificial Sequence
  Description of Artificial Sequence Synthetic peptide
QHLSTADQQYVTTQPYWATDAT

SEQ ID NO: 20 - protein, 22 aa, Artificial Sequence
  Description of Artificial Sequence Synthetic peptide
QHQSTADQQYTTTQPYWATDAT

SEQ ID NO: 21 - protein, 11 aa, Unknown
  Description of Unknown Mammalian CXCR4 peptide
ANWYFGNFLCK

SEQ ID NO: 22 - protein, 20 aa, Artificial Sequence
  Description of Artificial Sequence Synthetic peptide
AVHVTYTVNQYSSVQIQAFT

SEQ ID NO: 23 - protein, 20 aa, Artificial Sequence
  Description of Artificial Sequence Synthetic peptide
AVHTTYTVNQYSSVQIQAFT

SEQ ID NO: 24 - protein, 20 aa, Artificial Sequence
  Description of Artificial Sequence Synthetic peptide
AVHTTYTVNQYSSVQTQAFT

SEQ ID NO: 25 - protein, 20 aa, Artificial Sequence
  Description of Artificial Sequence Synthetic peptide
ATHTTYTVNQYSSVQTQAFT

SEQ ID NO: 26 - protein, 20 aa, Artificial Sequence
  Description of Artificial Sequence Synthetic peptide
ATHTIYTTNQYSSVQTQAFT

SEQ ID NO: 27 - protein, 20 aa, Artificial Sequence
  Description of Artificial Sequence Synthetic peptide
AVHTTYTTNQYSSVQTQAFT

SEQ ID NO: 28 - protein, 20 aa, Artificial Sequence
  Description of Artificial Sequence Synthetic peptide
ATHTTYTTNQYSSVQTQAFT

SEQ ID NO: 29 - protein, 20 aa, Artificial Sequence
  Description of Artificial Sequence Synthetic peptide
ATHTTYTTNQYSSTQTQAYT

SEQ ID NO: 30 - protein, 24 aa, Unknown
  Description of Unknown Mammalian CXCR4 peptide
SLDRYLAIVHATNSQRPRKLLAEK

SEQ ID NO: 31 - protein, 20 aa, Artificial Sequence
  Description of Artificial Sequence Synthetic peptide
VTYTGVWTPAQQQTIPDFIF

SEQ ID NO: 32 - protein, 20 aa, Artificial Sequence
  Description of Artificial Sequence Synthetic peptide
TTYTGTWIPAQQQTIPDFIF

SEQ ID NO: 33 - protein, 20 aa, Artificial Sequence
  Description of Artificial Sequence Synthetic peptide
TTYTGTWTPAQQQTIPDFIF

SEQ ID NO: 34 - protein, 20 aa, Artificial Sequence
  Description of Artificial Sequence Synthetic peptide
TTYTGTWTPAQQQTIPDFIY

SEQ ID NO: 35 - protein, 20 aa, Artificial Sequence
  Description of Artificial Sequence Synthetic peptide
TTYVGTWTPAQQQTTPDYIF

SEQ ID NO: 36 - protein, 20 aa, Artificial Sequence
  Description of Artificial Sequence Synthetic peptide
TTYVGTWTPAQQQTTPDFIY

SEQ ID NO: 37 - protein, 20 aa, Artificial Sequence
  Description of Artificial Sequence Synthetic peptide
TTYTGVWTPAQQQTTPDYTF

SEQ ID NO: 38 - protein, 20 aa, Artificial Sequence
  Description of Artificial Sequence Synthetic peptide
TTYTGTWTPAQQQTTPDYTY

SEQ ID NO: 39 - protein, 21 aa, Unknown
  Description of Unknown Mammalian CXCR4 peptide
ANVSEADDRYICDRFYPNDLW

SEQ ID NO: 40 - protein, 21 aa, Artificial Sequence
  Description of Artificial Sequence Synthetic peptide
VVVFQFQHTMVGQTQPGTTTQ

SEQ ID NO: 41 - protein, 21 aa, Artificial Sequence
  Description of Artificial Sequence Synthetic peptide
VVVFQFQHTMTGQTQPGTTTQ

SEQ ID NO: 42 - protein, 21 aa, Artificial Sequence
  Description of Artificial Sequence Synthetic peptide
VVVFQYQHTMTGQTQPGTTTQ

SEQ ID NO: 43 - protein, 21 aa, Artificial Sequence
  Description of Artificial Sequence Synthetic peptide
VVVYQYQHTMTGQTQPGTTTQ

SEQ ID NO: 44 - protein, 21 aa, Artificial Sequence
  Description of Artificial Sequence Synthetic peptide
TVVFQYQHTMTGQTQPGTTTQ

SEQ ID NO: 45 - protein, 21 aa, Artificial Sequence
  Description of Artificial Sequence Synthetic peptide
VVTFQYQHTMTGQTQPGTTTQ

SEQ ID NO: 46 - protein, 21 aa, Artificial Sequence
  Description of Artificial Sequence Synthetic peptide
TVVYQYQHTMTGQTQPGTTTQ

SEQ ID NO: 47 - protein, 21 aa, Artificial Sequence
  Description of Artificial Sequence Synthetic peptide
TTTYQYQHTMTGQTQPGTTTQ

SEQ ID NO: 48 - protein, 25 aa, Unknown
  Description of Unknown Mammalian CXCR4 peptide
SCYCIIISKLSHSKGHQKRKALKTT

SEQ ID NO: 49 - protein, 20 aa, Artificial Sequence
  Description of Artificial Sequence Synthetic peptide
VTQIQAFFACWQPYYTGTST

SEQ ID NO: 50 - protein, 20 aa, Artificial Sequence
  Description of Artificial Sequence Synthetic peptide
VIQIQAYFACWQPYYTGTST

SEQ ID NO: 51 - protein, 20 aa, Artificial Sequence
  Description of Artificial Sequence Synthetic peptide
VIQIQAYYACWQPYYTGTST

SEQ ID NO: 52 - protein, 20 aa, Artificial Sequence
  Description of Artificial Sequence Synthetic peptide
VIQTQAFYACWQPYYTGTST

SEQ ID NO: 53 - protein, 20 aa, Artificial Sequence
  Description of Artificial Sequence Synthetic peptide
VIQTQAYFACWQPYYTGTST

SEQ ID NO: 54 - protein, 20 aa, Artificial Sequence
  Description of Artificial Sequence Synthetic peptide
VTQIQAFYACWQPYYTGTST

SEQ ID NO: 55 - protein, 20 aa, Artificial Sequence
  Description of Artificial Sequence Synthetic peptide
VIQTQAYYACWQPYYTGTST

SEQ ID NO: 56 - protein, 20 aa, Artificial Sequence
  Description of Artificial Sequence Synthetic peptide
TTQTQAYYACWQPYYTGTST

SEQ ID NO: 57 - protein, 21 aa, Unknown
  Description of Unknown Mammalian CXCR4 peptide
DSFILLEIIKQGCEFENTVHK

SEQ ID NO: 58 - protein, 20 aa, Artificial Sequence
  Description of Artificial Sequence Synthetic peptide
WISITEAQAFFHCCLNPIQY

SEQ ID NO: 59 - protein, 20 aa, Artificial Sequence
  Description of Artificial Sequence Synthetic peptide
WISITEAQAFYHCCLNPIQY

SEQ ID NO: 60 - protein, 20 aa, Artificial Sequence
  Description of Artificial Sequence Synthetic peptide
WISITEAQAYFHCCQNPTLY

SEQ ID NO: 61 - protein, 20 aa, Artificial Sequence
  Description of Artificial Sequence Synthetic peptide
WISTTEALAFYHCCQNPTQY

SEQ ID NO: 62 - protein, 20 aa, Artificial Sequence
  Description of Artificial Sequence Synthetic peptide
WISTTEALAYFHCCQNPTQY

SEQ ID NO: 63 - protein, 20 aa, Artificial Sequence
  Description of Artificial Sequence Synthetic peptide
WISITEALAYYHCCQNPTQY

SEQ ID NO: 64 - protein, 20 aa, Artificial Sequence
  Description of Artificial Sequence Synthetic peptide
WISTTEALAYYHCCQNPTQY

SEQ ID NO: 65 - protein, 50 aa, Unknown
  Description of Unknown Mammalian CXCR4 polypeptide
AFLGAKFKTSAQHALTSVSRGSSLKILSKGKRGGHSSVSTESESSSFHSS

SEQ ID NO: 66 - protein, 355 aa, Unknown
  Description of Unknown Mammalian CX3CR1 polypeptide
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 67 - protein, 355 aa, Artificial Sequence
  Description of Artificial Sequence Synthetic polypeptide
MDQFPESVTENFEYDDLAEACYIGDIVVFGTVFQSTYYSTTYATGQTGNQQTTYAQTNSKKPKSVTDTYQQNQAQSDQQYTATQPYWTHYQINEKGLHNAMCKFTTAYYYTGYYGSTYYTTTTSTDRYLAIVLAANSMNNRTVQHGTTTSQGTWAAATQTAAPQYMYTKQKENECLGDYPEVLQEIWPVLRNVETNYQGYQQPQQTMSYCYYRTTQTQYSCKNHKKAKAIKQTQQTTTTYYQYWTPYNTMTYQETLKLYDFFPSCDMRKDLRLAQSTTETTAYSHCCQNPQTYAYAGEKFRRYLYHLYGKCLAVLCGRSVHVDFSSSESQRSRHGSVLSSNFTYHTSDGDALLLL

SEQ ID NO: 68 - protein, 31 aa, Unknown
  Description of Unknown Mammalian CX3CR1 polypeptide
MDQFPESVTENFEYDDLAEACYIGDIVVFGT

SEQ ID NO: 69 - protein, 28 aa, Artificial Sequence
  Description of Artificial Sequence Synthetic peptide
TYQSTYYSTTFATGQVGNQQVVFALTNS

SEQ ID NO: 70 - protein, 28 aa, Artificial Sequence
  Description of Artificial Sequence Synthetic peptide
TYQSTYYSTTYATGQVGNQQVVFALTNS

SEQ ID NO: 71 - protein, 28 aa, Artificial Sequence
  Description of Artificial Sequence Synthetic peptide
TYQSTYYSTTYATGQVGNQQVVFAQTNS

SEQ ID NO: 72 - protein, 28 aa, Artificial Sequence
  Description of Artificial Sequence Synthetic peptide
TYQSTYYSTTYATGQTGNLQVTFAQTNS

SEQ ID NO: 73 - protein, 28 aa, Artificial Sequence
  Description of Artificial Sequence Synthetic peptide
TYQSTYYSTTYATGQTGNQLVTFAQTNS

SEQ ID NO: 74 - protein, 28 aa, Artificial Sequence
  Description of Artificial Sequence Synthetic peptide
TYQSTYYSTTYATGQTGNQQVVFAQTNS

SEQ ID NO: 75 - protein, 28 aa, Artificial Sequence
  Description of Artificial Sequence Synthetic peptide
TYQSTYYSTTYATGQTGNLQVTYAQTNS

SEQ ID NO: 76 - protein, 28 aa, Artificial Sequence
  Description of Artificial Sequence Synthetic peptide
TYQSTYYSTTYATGQTGNQQTTYAQTNS

SEQ ID NO: 77 - protein, 10 aa, Unknown
  Description of Unknown Mammalian CX3CR1 peptide
KKPKSVTDIY

SEQ ID NO: 78 - protein, 21 aa, Artificial Sequence
  Description of Artificial Sequence Synthetic peptide
LLNQAQSDQLFVATQPFWTHY

SEQ ID NO: 79 - protein, 21 aa, Artificial Sequence
  Description of Artificial Sequence Synthetic peptide
LLNQAQSDQQFVATQPFWTHY

SEQ ID NO: 80 - protein, 21 aa, Artificial Sequence
  Description of Artificial Sequence Synthetic peptide
QQNLAQSDQQFVATQPFWTHY

SEQ ID NO: 81 - protein, 21 aa, Artificial Sequence
  Description of Artificial Sequence Synthetic peptide
LQNLAQSDQQYTATQPFWTHY

SEQ ID NO: 82 - protein, 21 aa, Artificial Sequence
  Description of Artificial Sequence Synthetic peptide
QLNLAQSDQQYTATQPFWTHY

SEQ ID NO: 83 - protein, 21 aa, Artificial Sequence
  Description of Artificial Sequence Synthetic peptide
LLNQAQSDQQFTATQPYWTHY

SEQ ID NO: 84 - protein, 21 aa, Artificial Sequence
  Description of Artificial Sequence Synthetic peptide
QQNLAQSDQQFTATQPYWTHY

SEQ ID NO: 85 - protein, 21 aa, Artificial Sequence
  Description of Artificial Sequence Synthetic peptide
QQNQAQSDQQYTATQPYWTHY

SEQ ID NO: 86 - protein, 13 aa, Unknown
  Description of Unknown Mammalian CX3CR1 peptide
LINEKGLHNAMCK

SEQ ID NO: 87 - protein, 22 aa, Artificial Sequence
  Description of Artificial Sequence Synthetic peptide
YTTAYYYTGYYGSTYYTTTTST

SEQ ID NO: 88 - protein, 17 aa, Unknown
  Description of Unknown Mammalian CX3CR1 peptide
DRYLAIVLAANSMNNRT

SEQ ID NO: 89 - protein, 25 aa, Artificial Sequence
  Description of Artificial Sequence Synthetic peptide
VQHGTTTSQGTWAAATQVAAPQFMF

SEQ ID NO: 90 - protein, 25 aa, Artificial Sequence
  Description of Artificial Sequence Synthetic peptide
VQHGVTTSQGTWAAATQTAAPQFMF

SEQ ID NO: 91 - protein, 25 aa, Artificial Sequence
  Description of Artificial Sequence Synthetic peptide
VQHGTTTSQGVWAAATQTAAPQFMY

SEQ ID NO: 92 - protein, 25 aa, Artificial Sequence
  Description of Artificial Sequence Synthetic peptide
VQHGTTTSQGTWAAAIQTAAPQFMY

SEQ ID NO: 93 - protein, 25 aa, Artificial Sequence
  Description of Artificial Sequence Synthetic peptide
VQHGTTTSQGTWAAATQTAAPQFMF

SEQ ID NO: 94 - protein, 25 aa, Artificial Sequence
  Description of Artificial Sequence Synthetic peptide
VQHGTTISQGTWAAATQTAAPQYMF

SEQ ID NO: 95 - protein, 25 aa, Artificial Sequence
  Description of Artificial Sequence Synthetic peptide
VQHGTTTSQGTWAAATQTAAPQFMY

SEQ ID NO: 96 - protein, 25 aa, Artificial Sequence
  Description of Artificial Sequence Synthetic peptide
TQHGTTTSQGTWAAATQTAAPQYMY

SEQ ID NO: 97 - protein, 28 aa, Unknown
  Description of Unknown Mammalian CX3CR1 peptide
TKQKENECLGDYPEVLQEIWPVLRNVET

SEQ ID NO: 98 - protein, 20 aa, Artificial Sequence
  Description of Artificial Sequence Synthetic peptide
NFLGFQQPQQIMSYCYFRIT

SEQ ID NO: 99 - protein, 20 aa, Artificial Sequence
  Description of Artificial Sequence Synthetic peptide
NFQGFLQPQQTMSYCYFRIT

SEQ ID NO: 100 - protein, 20 aa, Artificial Sequence
  Description of Artificial Sequence Synthetic peptide
NFQGFLQPQQTMSYCYFRTT

SEQ ID NO: 101 - protein, 20 aa, Artificial Sequence
  Description of Artificial Sequence Synthetic peptide
NFQGFQQPQQTMSYCYYRIT

SEQ ID NO: 102 - protein, 20 aa, Artificial Sequence
  Description of Artificial Sequence Synthetic peptide
NFQGFLQPQQTMSYCYYRTT

SEQ ID NO: 103 - protein, 20 aa, Artificial Sequence
  Description of Artificial Sequence Synthetic peptide
NFQGYLQPQQTMSYCYFRTT

SEQ ID NO: 104 - protein, 20 aa, Artificial Sequence
  Description of Artificial Sequence Synthetic peptide
NYQGFQQPQQTMSYCYFRTT

SEQ ID NO: 105 - protein, 20 aa, Artificial Sequence
  Description of Artificial Sequence Synthetic peptide
NYQGYQQPQQTMSYCYYRTT

SEQ ID NO: 106 - protein, 16 aa, Unknown
  Description of Unknown Mammalian CX3CR1 peptide
QTLFSCKNHKKAKAIK

SEQ ID NO: 107 - protein, 25 aa, Artificial Sequence
  Description of Artificial Sequence Synthetic peptide
LIQQTTTTFYQFWTPYNTMTFQETL

SEQ ID NO: 108 - protein, 25 aa, Artificial Sequence
  Description of Artificial Sequence Synthetic peptide
LIQQTTTTFYQYWTPYNVMTFQETQ

SEQ ID NO: 109 - protein, 25 aa, Artificial Sequence
  Description of Artificial Sequence Synthetic peptide
LIQQTTTTYYQFWTPYNTMTFQETQ

SEQ ID NO: 110 - protein, 25 aa, Artificial Sequence
  Description of Artificial Sequence Synthetic peptide
QIQQTTTTFYQYWTPYNTMTFQETQ

SEQ ID NO: 111 - protein, 25 aa, Artificial Sequence
  Description of Artificial Sequence Synthetic peptide
LTQQTTTTYYQFWTPYNTMTFQETQ

SEQ ID NO: 112 - protein, 25 aa, Artificial Sequence
  Description of Artificial Sequence Synthetic peptide
QIQQTTTTFFQYWTPYNTMTYQETQ

SEQ ID NO: 113 - protein, 25 aa, Artificial Sequence
  Description of Artificial Sequence Synthetic peptide
QIQQTTTTFYQYWTPYNTMTYQETQ

SEQ ID NO: 114 - protein, 25 aa, Artificial Sequence
  Description of Artificial Sequence Synthetic peptide
QTQQTTTTYYQYWTPYNTMTYQETQ

SEQ ID NO: 115 - protein, 17 aa, Unknown
  Description of Unknown Mammalian CX3CR1 peptide
KLYDFFPSCDMRKDLRL

SEQ ID NO: 116 - protein, 24 aa, Artificial Sequence
  Description of Artificial Sequence Synthetic peptide
ALSVTETVAFSHCCQNPQIYAFAG

SEQ ID NO: 117 - protein, 24 aa, Artificial Sequence
  Description of Artificial Sequence Synthetic peptide
AQSVTETTAFSHCCQNPLIYAFAG

SEQ ID NO: 118 - protein, 24 aa, Artificial Sequence
  Description of Artificial Sequence Synthetic peptide
ALSVTETVAFSHCCQNPQTYAYAG

SEQ ID NO: 119 - protein, 24 aa, Artificial Sequence
  Description of Artificial Sequence Synthetic peptide
AQSVTETTAFSHCCQNPQIYAYAG

SEQ ID NO: 120 - protein, 24 aa, Artificial Sequence
  Description of Artificial Sequence Synthetic peptide
ALSVTETTAFSHCCQNPQTYAYAG

SEQ ID NO: 121 - protein, 24 aa, Artificial Sequence
  Description of Artificial Sequence Synthetic peptide
ALSTTETTAYSHCCQNPQIYAFAG

SEQ ID NO: 122 - protein, 24 aa, Artificial Sequence
  Description of Artificial Sequence Synthetic peptide
ALSVTETTAYSHCCQNPQTYAYAG

SEQ ID NO: 123 - protein, 24 aa, Artificial Sequence
  Description of Artificial Sequence Synthetic peptide
AQSTTETTAYSHCCQNPQTYAYAG

SEQ ID NO: 124 - protein, 58 aa, Unknown
  Description of Unknown Mammalian CX3CR1 polypeptide
EKFRRYLYHLYGKCLAVLCGRSVHVDFSSSESQRSRHGSVLSSNFTYHTSDGDALLLL

SEQ ID NO: 125 - protein, 373 aa, Unknown
  Description of Unknown Mammalian CCR3 polypeptide
MPFGIRMLLRAHKPGRSEMTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 126 - protein, 373 aa, Artificial Sequence
  Description of Artificial Sequence Synthetic polypeptide
MPFGIRMLLRAHKPGRSEMTTSLDTVETFGTTSYYDDVGLLCEKADTRALMAQFVPPQYSQTYTTGQQGNTTTTMTQTKYRRLRIMTNTYQQNQATSDQQYQTTQPYWTHYVRGHNWVFGHGMCKLLSGFYHTGLYSETYYTTQQTTDRYQATTHATYAQRARTVTFGTTTSTTTWGQATQAAQPEYTYYETEELFEETLCSALYPEDTVYSWRHFHTLRMTTYCQTQPQQTMATCYTGTTKTLLRCPSKKKYKAIRQTYTTMATYYTYWTPYNTATQQSSYQSILFGNDCERSKHLDQTMQTTETTAYSHCCMNPTTYAYTGERFRKYLRHFFHRHLLMHLGRYIPFLPSEKLERTSSVSPSTAEPELSIVF

SEQ ID NO: 127 - protein, 34 aa, Unknown
  Description of Unknown Mammalian CCR3 polypeptide
MTTSLDTVETFGTTSYYDDVGLLCEKADTRALMA

SEQ ID NO: 128 - protein, 28 aa, Artificial Sequence
  Description of Artificial Sequence Synthetic peptide
QFVPPQYSQTFTTGQQGNVTVTMTQIKY

SEQ ID NO: 129 - protein, 28 aa, Artificial Sequence
  Description of Artificial Sequence Synthetic peptide
QFVPPQYSQTFTTGQQGNTTVTMTQIKY

SEQ ID NO: 130 - protein, 28 aa, Artificial Sequence
  Description of Artificial Sequence Synthetic peptide
QFVPPQYSQTYTTGQQGNTTVTMTQIKY

SEQ ID NO: 131 - protein, 28 aa, Artificial Sequence
  Description of Artificial Sequence Synthetic peptide
QFTPPQYSQTYTTGQQGNVTTTMTQIKY

SEQ ID NO: 132 - protein, 28 aa, Artificial Sequence
  Description of Artificial Sequence Synthetic peptide
QFTPPQYSQTYTTGQQGNTVTTMTQIKY

SEQ ID NO: 133 - protein, 28 aa, Artificial Sequence
  Description of Artificial Sequence Synthetic peptide
QFTPPQYSQTYTTGQQGNTTVTMTQIKY

SEQ ID NO: 134 - protein, 28 aa, Artificial Sequence
  Description of Artificial Sequence Synthetic peptide
QFTPPQYSQTYTTGQQGNTTTTMTQIKY

SEQ ID NO: 135 - protein, 28 aa, Artificial Sequence
  Description of Artificial Sequence Synthetic peptide
QYTPPQYSQTYTTGQQGNTTTTMTQTKY

SEQ ID NO: 136 - protein, 10 aa, Unknown
  Description of Unknown Mammalian CCR3 peptide
RRLRIMTNIY

SEQ ID NO: 137 - protein, 21 aa, Artificial Sequence
  Description of Artificial Sequence Synthetic peptide
LLNQATSDQQFQVTQPFWIHY

SEQ ID NO: 138 - protein, 21 aa, Artificial Sequence
  Description of Artificial Sequence Synthetic peptide
LQNQAISDQLFQTTQPFWTHY

SEQ ID NO: 139 - protein, 21 aa, Artificial Sequence
  Description of Artificial Sequence Synthetic peptide
QQNLAISDQQFQTTQPFWTHY

SEQ ID NO: 140 - protein, 21 aa, Artificial Sequence
  Description of Artificial Sequence Synthetic peptide
QLNQAISDQQFQTTQPYWTHY

SEQ ID NO: 141 - protein, 21 aa, Artificial Sequence
  Description of Artificial Sequence Synthetic peptide
QQNLAISDQQYQVTQPYWTHY

SEQ ID NO: 142 - protein, 21 aa, Artificial Sequence
  Description of Artificial Sequence Synthetic peptide
LQNQATSDQLFQTTQPYWTHY

SEQ ID NO: 143 - protein, 21 aa, Artificial Sequence
  Description of Artificial Sequence Synthetic peptide
QQNQAISDQQYQVTQPYWTHY

SEQ ID NO: 144 - protein, 21 aa, Artificial Sequence
  Description of Artificial Sequence Synthetic peptide
QQNQATSDQQYQTTQPYWTHY

SEQ ID NO: 145 - protein, 14 aa, Unknown
  Description of Unknown Mammalian CCR3 peptide
VRGHNWVFGHGMCK

SEQ ID NO: 146 - protein, 22 aa, Artificial Sequence
  Description of Artificial Sequence Synthetic peptide
LQSGFYHTGQYSETFFTTQQTT

SEQ ID NO: 147 - protein, 22 aa, Artificial Sequence
  Description of Artificial Sequence Synthetic peptide
QLSGFYHTGQYSETFFTTQQTT

SEQ ID NO: 148 - protein, 22 aa, Artificial Sequence
  Description of Artificial Sequence Synthetic peptide
QLSGFYHTGQYSETFYTTQQTT

SEQ ID NO: 149 - protein, 22 aa, Artificial Sequence
  Description of Artificial Sequence Synthetic peptide
QLSGFYHTGQYSETYFTTQQTT

SEQ ID NO: 150 - protein, 22 aa, Artificial Sequence
  Description of Artificial Sequence Synthetic peptide
QLSGYYHTGQYSETFFTTQQTT

SEQ ID NO: 151 - protein, 22 aa, Artificial Sequence
  Description of Artificial Sequence Synthetic peptide
QQSGFYHTGQYSETFFTTQQTT

SEQ ID NO: 152 - protein, 22 aa, Artificial Sequence
  Description of Artificial Sequence Synthetic peptide
QQSGFYHTGQYSETFYTTQQTT

SEQ ID NO: 153 - protein, 22 aa, Artificial Sequence
  Description of Artificial Sequence Synthetic peptide
QQSGYYHTGQYSETYYTTQQTT

SEQ ID NO: 154 - protein, 17 aa, Unknown
  Description of Unknown Mammalian CCR3 peptide
DRYLAIVHAVFALRART

SEQ ID NO: 155 - protein, 25 aa, Artificial Sequence
  Description of Artificial Sequence Synthetic peptide
TTFGTTTSTVTWGQAVQAAQPEFIF

SEQ ID NO: 156 - protein, 25 aa, Artificial Sequence
  Description of Artificial Sequence Synthetic peptide
TTFGTTTSTTTWGQAVQAAQPEFIF

SEQ ID NO: 157 - protein, 25 aa, Artificial Sequence
  Description of Artificial Sequence Synthetic peptide
TTYGTTTSTTTWGQAVQAAQPEFIF

SEQ ID NO: 158 - protein, 25 aa, Artificial Sequence
  Description of Artificial Sequence Synthetic peptide
TTYGTTTSTTTWGQAVQAAQPEFTF

SEQ ID NO: 159 - protein, 25 aa, Artificial Sequence
  Description of Artificial Sequence Synthetic peptide
TTYGTTTSTTTWGQATQAAQPEFIF

SEQ ID NO: 160 - protein, 25 aa, Artificial Sequence
  Description of Artificial Sequence Synthetic peptide
TTFGTTTSTTTWGQATQAAQPEFIY

SEQ ID NO: 161 - protein, 25 aa, Artificial Sequence
  Description of Artificial Sequence Synthetic peptide
TTYGTTTSTTTWGQATQAAQPEFIY

SEQ ID NO: 162 - protein, 25 aa, Artificial Sequence
  Description of Artificial Sequence Synthetic peptide
TTYGTTTSTTTWGQATQAAQPEYTY

SEQ ID NO: 163 - protein, 32 aa, Unknown
  Description of Unknown Mammalian CCR3 polypeptide
YETEELFEETLCSALYPEDTVYSWRHFHTLRM

SEQ ID NO: 164 - protein, 20 aa, Artificial Sequence
  Description of Artificial Sequence Synthetic peptide
TIFCQVQPQQTMATCYTGTT

SEQ ID NO: 165 - protein, 20 aa, Artificial Sequence
  Description of Artificial Sequence Synthetic peptide
TIFCQTQPQQVMATCYTGTT

SEQ ID NO: 166 - protein, 20 aa, Artificial Sequence
  Description of Artificial Sequence Synthetic peptide
TIFCQTQPQQTMATCYTGIT

SEQ ID NO: 167 - protein, 20 aa, Artificial Sequence
  Description of Artificial Sequence Synthetic peptide
TIFCQTQPQQTMATCYTGTI

SEQ ID NO: 168 - protein, 20 aa, Artificial Sequence
  Description of Artificial Sequence Synthetic peptide
TTFCQVQPQQVMATCYTGTT

SEQ ID NO: 169 - protein, 20 aa, Artificial Sequence
  Description of Artificial Sequence Synthetic peptide
TIYCQVQPQQVMATCYTGTT

SEQ ID NO: 170 - protein, 20 aa, Artificial Sequence
  Description of Artificial Sequence Synthetic peptide
TIFCQTQPQQTMATCYTGTT

SEQ ID NO: 171 - protein, 20 aa, Artificial Sequence
  Description of Artificial Sequence Synthetic peptide
TTYCQTQPQQTMATCYTGTT

SEQ ID NO: 172 - protein, 16 aa, Unknown
  Description of Unknown Mammalian CCR3 peptide
KTLLRCPSKKKYKAIR

SEQ ID NO: 173 - protein, 25 aa, Artificial Sequence
  Description of Artificial Sequence Synthetic peptide
QTYTTMATYYTYWTPYNTATQQSSY

SEQ ID NO: 174 - protein, 17 aa, Unknown
  Description of Unknown Mammalian CCR3 peptide
QSILFGNDCERSKHLDL

SEQ ID NO: 175 - protein, 24 aa, Artificial Sequence
  Description of Artificial Sequence Synthetic peptide
VMQVTEVTAYSHCCMNPVTYAFTG

SEQ ID NO: 176 - protein, 24 aa, Artificial Sequence
  Description of Artificial Sequence Synthetic peptide
VMQVTEVTAYSHCCMNPTTYAYVG

SEQ ID NO: 177 - protein, 24 aa, Artificial Sequence
  Description of Artificial Sequence Synthetic peptide
VMLTTEVTAYSHCCMNPTTYAFTG

SEQ ID NO: 178 - protein, 24 aa, Artificial Sequence
  Description of Artificial Sequence Synthetic peptide
VMQVTETTAYSHCCMNPVTYAYTG

SEQ ID NO: 179 - protein, 24 aa, Artificial Sequence
  Description of Artificial Sequence Synthetic peptide
TMQVTETIAYSHCCMNPTTYAFTG

SEQ ID NO: 180 - protein, 24 aa, Artificial Sequence
  Description of Artificial Sequence Synthetic peptide
TMQVTETTAYSHCCMNPTTYAFVG

SEQ ID NO: 181 - protein, 24 aa, Artificial Sequence
  Description of Artificial Sequence Synthetic peptide
VMQTTETIAYSHCCMNPTTYAYTG

SEQ ID NO: 182 - protein, 24 aa, Artificial Sequence
  Description of Artificial Sequence Synthetic peptide
TMQTTETTAYSHCCMNPTTYAYTG

SEQ ID NO: 183 - protein, 50 aa, Unknown
  Description of Unknown Mammalian CCR3 polypeptide
ERFRKYLRHFFHRHLLMHLGRYIPFLPSEKLERTSSVSPSTAEPELSIVF

SEQ ID NO: 184 - protein, 352 aa, Unknown [sequence omitted]

SEQ ID NO: 185 - protein, 352 aa, Artificial Sequence
  Description of Artificial Sequence Synthetic polypeptide
MDYQVSSPIYDINYYTSEPCQKINVKQIAARLLPPQYSQTYTYGYTGNMQTTQTQTNCKRLKSMTDTYQQNQATSDQYYQQTTPYWAHYAAAQWDFGNTMCQQQTGQYYTGYYSGTYYTTQQTTDRYLAVVHAVFALKARTVTYGTTTSTTTWTTATYASQPGTTYTRSQKEGLHYTCSSHFPYSQYQFWKNFQTLKTTTQGQTQPQQTMTTCYSGTQKTQLRCRNEKKRHRAVRQTYTTMTTYYQYWAPYNTTQQQNTFQEFFGLNNCSSSNRLDQAMQVTETLGMTHCCTNPTTYAYTGEKYRNYQQTYYQKHIAKRFCKCCSIFQQEAPERASSVYTRSTGEQEISVGL

SEQ ID NO: 186 - protein, 30 aa, Unknown
  Description of Unknown Mammalian CCR5 polypeptide
MDYQVSSPIYDINYYTSEPCQKINVKQIAA

SEQ ID NO: 187 - protein, 28 aa, Artificial Sequence
  Description of Artificial Sequence Synthetic peptide
RLQPPQYSQTFTFGFTGNMQVTQTQINC

SEQ ID NO: 188 - protein, 28 aa, Artificial Sequence
  Description of Artificial Sequence Synthetic peptide
RLQPPQYSQTFTFGYTGNMQVTQTQINC

SEQ ID NO: 189 - protein, 28 aa, Artificial Sequence
  Description of Artificial Sequence Synthetic peptide
RQQPPQYSQTFTFGFTGNMQTTQTQINC

SEQ ID NO: 190 - protein, 28 aa, Artificial Sequence
  Description of Artificial Sequence Synthetic peptide
RQQPPQYSQTFTYGFTGNMQTTQTQINC

SEQ ID NO: 191 - protein, 28 aa, Artificial Sequence
  Description of Artificial Sequence Synthetic peptide
RQQPPQYSQTYTFGFTGNMQTTQTQINC

SEQ ID NO: 192 - protein, 28 aa, Artificial Sequence
  Description of Artificial Sequence Synthetic peptide
RQQPPQYSQTFTFGYTGNMQTTQTQINC

SEQ ID NO: 193 - protein, 28 aa, Artificial Sequence
  Description of Artificial Sequence Synthetic peptide
RQQPPQYSQTYTFGYTGNMQTTQTQINC

SEQ ID NO: 194 - protein, 28 aa, Artificial Sequence
  Description of Artificial Sequence Synthetic peptide
RQQPPQYSQTYTYGYTGNMQTTQTQTNC

SEQ ID NO: 195 - protein, 10 aa, Unknown
  Description of Unknown Mammalian CCR5 peptide
KRLKSMTDIY

SEQ ID NO: 196 - protein, 21 aa, Artificial Sequence
  Description of Artificial Sequence Synthetic peptide
LQNQAISDQFFQQTVPFWAHY

SEQ ID NO: 197 - protein, 21 aa, Artificial Sequence
  Description of Artificial Sequence Synthetic peptide
LQNQAISDQFFQQTTPFWAHY

SEQ ID NO: 198 - protein, 21 aa, Artificial Sequence
  Description of Artificial Sequence Synthetic peptide
LQNQAISDQFFQQTTPYWAHY

SEQ ID NO: 199 - protein, 21 aa, Artificial Sequence
  Description of Artificial Sequence Synthetic peptide
LQNQAISDQFYQQTTPYWAHY

SEQ ID NO: 200 - protein, 21 aa, Artificial Sequence
  Description of Artificial Sequence Synthetic peptide
LQNQAISDQYFQQTTPYWAHY

SEQ ID NO: 201 - protein, 21 aa, Artificial Sequence
  Description of Artificial Sequence Synthetic peptide
LQNQATSDQFFQQTTPYWAHY

SEQ ID NO: 202 - protein, 21 aa, Artificial Sequence
  Description of Artificial Sequence Synthetic peptide
LQNQAISDQYYQQTTPYWAHY

SEQ ID NO: 203 - protein, 21 aa, Artificial Sequence
  Description of Artificial Sequence Synthetic peptide
QQNQATSDQYYQQTTPYWAHY

SEQ ID NO: 204 - protein, 13 aa, Unknown
  Description of Unknown Mammalian CCR5 peptide
AAAQWDFGNTMCQ

SEQ ID NO: 205 - protein, 22 aa, Artificial Sequence
  Description of Artificial Sequence Synthetic peptide
QQTGQYFTGYYSGTYYTTQQTT

SEQ ID NO: 206 - protein, 22 aa, Artificial Sequence
  Description of Artificial Sequence Synthetic peptide
QQTGQYYTGYYSGTYYTTQQTT

SEQ ID NO: 207 - protein, 17 aa, Unknown
  Description of Unknown Mammalian CCR5 peptide
DRYLAVVHAVFALKART

SEQ ID NO: 208 - protein, 25 aa, Artificial Sequence
  Description of Artificial Sequence Synthetic peptide
TTYGTTTSTTTWTTATYASQPGTTY

SEQ ID NO: 209 - protein, 32 aa, Unknown
  Description of Unknown Mammalian CCR5 polypeptide
TRSQKEGLHYTCSSHFPYSQYQFWKNFQTLKI

SEQ ID NO: 210 - protein, 20 aa, Artificial Sequence
  Description of Artificial Sequence Synthetic peptide
VIQGQVQPQQVMVTCYSGIQ

SEQ ID NO: 211 - protein, 20 aa, Artificial Sequence
  Description of Artificial Sequence Synthetic peptide
VIQGQVQPQQVMTTCYSGIQ

SEQ ID NO: 212 - protein, 20 aa, Artificial Sequence
  Description of Artificial Sequence Synthetic peptide
VIQGQVQPQQTMTTCYSGIQ

SEQ ID NO: 213 - protein, 20 aa, Artificial Sequence
  Description of Artificial Sequence Synthetic peptide
VTQGQVQPQQTMVTCYSGTQ

SEQ ID NO: 214 - protein, 20 aa, Artificial Sequence
  Description of Artificial Sequence Synthetic peptide
TIQGQVQPQQVMTTCYSGTQ

SEQ ID NO: 215 - protein, 20 aa, Artificial Sequence
  Description of Artificial Sequence Synthetic peptide
TIQGQVQPQQTMVTCYSGTQ

SEQ ID NO: 216 - protein, 20 aa, Artificial Sequence
  Description of Artificial Sequence Synthetic peptide
TTQGQVQPQQVMTTCYSGTQ

SEQ ID NO: 217 - protein, 20 aa, Artificial Sequence
  Description of Artificial Sequence Synthetic peptide
TTQGQTQPQQTMTTCYSGTQ

SEQ ID NO: 218 - protein, 17 aa, Unknown
  Description of Unknown Mammalian CCR5 peptide
KTLLRCRNEKKRHRAVR

SEQ ID NO: 219 - protein, 25 aa, Artificial Sequence
  Description of Artificial Sequence Synthetic peptide
QTFTTMTTYYQFWAPYNIVQQLNTF

SEQ ID NO: 220 - protein, 25 aa, Artificial Sequence
  Description of Artificial Sequence Synthetic peptide
QTFTTMTTYYQFWAPYNTVQQLNTF

SEQ ID NO: 221 - protein, 25 aa, Artificial Sequence
  Description of Artificial Sequence Synthetic peptide
QTFTTMTTYYQYWAPYNTVQQLNTF

SEQ ID NO: 222 - protein, 25 aa, Artificial Sequence
  Description of Artificial Sequence Synthetic peptide
QTFTTMTTYYQYWAPYNTVQQQNTF

SEQ ID NO: 223 - protein, 25 aa, Artificial Sequence
  Description of Artificial Sequence Synthetic peptide
QTYTTMTTYYQYWAPYNTVQQLNTF

SEQ ID NO: 224 - protein, 25 aa, Artificial Sequence
  Description of Artificial Sequence Synthetic peptide
QTFTTMTTYYQYWAPYNTTQQLNTF

SEQ ID NO: 225 - protein, 25 aa, Artificial Sequence
  Description of Artificial Sequence Synthetic peptide
QTYTTMTTYYQYWAPYNTVQQQNTF

SEQ ID NO: 226 - protein, 17 aa, Unknown
  Description of Unknown Mammalian CCR5 peptide
QEFFGLNNCSSSNRLDQ

SEQ ID NO: 227 - protein, 24 aa, Artificial Sequence
  Description of Artificial Sequence Synthetic peptide
AMQVTETQGMTHCCINPIIYAFVG

SEQ ID NO: 228 - protein, 24 aa, Artificial Sequence
  Description of Artificial Sequence Synthetic peptide
AMQVTETLGMTHCCTNPIIYAFTG

SEQ ID NO: 229 - protein, 24 aa, Artificial Sequence
  Description of Artificial Sequence Synthetic peptide
AMQVTETQGMTHCCINPTIYAYVG

SEQ ID NO: 230 - protein, 24 aa, Artificial Sequence
  Description of Artificial Sequence Synthetic peptide
AMQTTETQGMTHCCINPITYAFTG

SEQ ID NO: 231 - protein, 24 aa, Artificial Sequence
  Description of Artificial Sequence Synthetic peptide
AMQTTETQGMTHCCINPTIYAFTG

SEQ ID NO: 232 - protein, 24 aa, Artificial Sequence
  Description of Artificial Sequence Synthetic peptide
AMQVTETQGMTHCCTNPTIYAYVG

SEQ ID NO: 233 - protein, 24 aa, Artificial Sequence
  Description of Artificial Sequence Synthetic peptide
AMQTTETQGMTHCCINPTTYAYVG

SEQ ID NO: 234 - protein, 24 aa, Artificial Sequence
  Description of Artificial Sequence Synthetic peptide
AMQTTETQGMTHCCTNPTTYAYTG

SEQ ID NO: 235 - protein, 51 aa, Unknown
  Description of Unknown Mammalian CCR5 polypeptide
EKFRNYLLVFFQKHIAKRFCKCCSIFQQEAPERASSVYTRSTGEQEISVGL

SEQ ID NO: 236 - protein, 27 aa, Artificial Sequence
  Description of Artificial Sequence Synthetic peptide
AFLPALYSQQFQQGQQGNGAVAATQLS

SEQ ID NO: 237 - protein, 27 aa, Artificial Sequence
  Description of Artificial Sequence Synthetic peptide
AFQPALYSQQFQQGQQGNGAVAAVQQS

SEQ ID NO: 238 - protein, 27 aa, Artificial Sequence
  Description of Artificial Sequence Synthetic peptide
AFQPAQYSQQFLQGQQGNGAVAATQQS

SEQ ID NO: 239 - protein, 27 aa, Artificial Sequence
  Description of Artificial Sequence Synthetic peptide
AYQPALYSLQYQQGQQGNGATAAVQQS

SEQ ID NO: 240 - protein, 27 aa, Artificial Sequence
  Description of Artificial Sequence Synthetic peptide
AYQPALYSQLFQQGQQGNGATAATQQS

SEQ ID NO: 241 - protein, 27 aa, Artificial Sequence
  Description of Artificial Sequence Synthetic peptide
AFQPALYSLQYQQGQQGNGATAATQQS

SEQ ID NO: 242 - protein, 27 aa, Artificial Sequence
  Description of Artificial Sequence Synthetic peptide
AYQPAQYSLQYQQGQQGNGATAAVQQS

SEQ ID NO: 243 - protein, 27 aa, Artificial Sequence
  Description of Artificial Sequence Synthetic peptide
AYQPAQYSQQYQQGQQGNGATAATQQS

SEQ ID NO: 244 - protein, 9 aa, Unknown
  Description of Unknown Mammalian CXCR3 peptide
RRTALSSTD

SEQ ID NO: 245 - protein, 21 aa, Artificial Sequence
  Description of Artificial Sequence Synthetic peptide
TFLQHLAVADTQQVQTLPQWA

SEQ ID NO: 246 - protein, 21 aa, Artificial Sequence
  Description of Artificial Sequence Synthetic peptide
TFLQHQAVADTQLVQTQPQWA

SEQ ID NO: 247 - protein, 21 aa, Artificial Sequence
  Description of Artificial Sequence Synthetic peptide
TFQQHLAVADTQQVQTQPQWA

SEQ ID NO: 248 - protein, 21 aa, Artificial Sequence
  Description of Artificial Sequence Synthetic peptide
TYLQHQAVADTQQVQTQPQWA

SEQ ID NO: 249 - protein, 21 aa, Artificial Sequence
  Description of Artificial Sequence Synthetic peptide
TYQLHQAVADTQQVQTQPQWA

SEQ ID NO: 250 - protein, 21 aa, Artificial Sequence
  Description of Artificial Sequence Synthetic peptide
TYQQHLAVADTQQVQTQPQWA

SEQ ID NO: 251 - protein, 21 aa, Artificial Sequence
  Description of Artificial Sequence Synthetic peptide
TYQQHQAVADTQQVQTQPQWA

SEQ ID NO: 252 - protein, 21 aa, Artificial Sequence
  Description of Artificial Sequence Synthetic peptide
TYQQHQATADTQQTQTQPQWA

SEQ ID NO: 253 - protein, 15 aa, Unknown
  Description of Unknown Mammalian CXCR3 peptide
VDAAVQWVFGSGLCK

SEQ ID NO: 254 - protein, 22 aa, Artificial Sequence
  Description of Artificial Sequence Synthetic peptide
TAGAQYNTNFYAGAQQQACISF

SEQ ID NO: 255 - protein, 22 aa, Artificial Sequence
  Description of Artificial Sequence Synthetic peptide
TAGAQYNTNFYAGAQLQACTSF

SEQ ID NO: 256 - protein, 22 aa, Artificial Sequence
  Description of Artificial Sequence Synthetic peptide
TAGAQYNTNFYAGAQQLACTSF

SEQ ID NO: 257 - protein, 22 aa, Artificial Sequence
  Description of Artificial Sequence Synthetic peptide
TAGAQFNTNYYAGAQQQACISF

SEQ ID NO: 258 - protein, 22 aa, Artificial Sequence
  Description of Artificial Sequence Synthetic peptide
TAGAQYNTNYYAGAQQQACISF

SEQ ID NO: 259 - protein, 22 aa, Artificial Sequence
  Description of Artificial Sequence Synthetic peptide
TAGAQYNTNYYAGAQLQACTSF

SEQ ID NO: 260 - protein, 22 aa, Artificial Sequence
  Description of Artificial Sequence Synthetic peptide
TAGAQYNTNYYAGAQQLACTSF

SEQ ID NO: 261 - protein, 22 aa, Artificial Sequence
  Description of Artificial Sequence Synthetic peptide
TAGAQYNTNYYAGAQQQACTSY

SEQ ID NO: 262 - protein, 22 aa, Unknown
  Description of Unknown Mammalian CXCR3 peptide
DRYLNIVHATQLYRRGPPARVT

SEQ ID NO: 263 - protein, 20 aa, Artificial Sequence
  Description of Artificial Sequence Synthetic peptide
LTCQAVWGQCQQFAQPDFIF

SEQ ID NO: 264 - protein, 20 aa, Artificial Sequence
  Description of Artificial Sequence Synthetic peptide
QTCQAVWGQCQQFAQPDFIF

SEQ ID NO: 265 - protein, 20 aa, Artificial Sequence
  Description of Artificial Sequence Synthetic peptide
QTCQATWGQCQQFAQPDFIF

SEQ ID NO: 266 - protein, 20 aa, Artificial Sequence
  Description of Artificial Sequence Synthetic peptide
QTCQATWGQCQQYAQPDFIF

SEQ ID NO: 267 - protein, 20 aa, Artificial Sequence
  Description of Artificial Sequence Synthetic peptide
QTCQATWGQCQQFAQPDFTF

SEQ ID NO: 268 - protein, 20 aa, Artificial Sequence
  Description of Artificial Sequence Synthetic peptide
QTCQATWGQCQQFAQPDYIF

SEQ ID NO: 269 - protein, 20 aa, Artificial Sequence
  Description of Artificial Sequence Synthetic peptide
QTCQATWGQCQQYAQPDYIF

SEQ ID NO: 270 - protein, 20 aa, Artificial Sequence
  Description of Artificial Sequence Synthetic peptide
QTCQATWGQCQQYAQPDYTY

SEQ ID NO: 271 - protein, 23 aa, Unknown
  Description of Unknown Mammalian CXCR3 peptide
LSAHHDERLNATHCQYNFPQVGR

SEQ ID NO: 272 - protein, 21 aa, Artificial Sequence
  Description of Artificial Sequence Synthetic peptide
TAQRTQQQTAGYQQPQQTMAY

SEQ ID NO: 273 - protein, 22 aa, Unknown
  Description of Unknown Mammalian CXCR3 peptide
CYAHILAVLLVSRGQRRLRAMR

SEQ ID NO: 274 - protein, 22 aa, Artificial Sequence
  Description of Artificial Sequence Synthetic peptide
QVTTTTVAFAQCWTPYHQVVQV

SEQ ID NO: 275 - protein, 22 aa, Artificial Sequence
  Description of Artificial Sequence Synthetic peptide
QVTTTTVAFAQCWTPYHQTVQV

SEQ ID NO: 276 - protein, 22 aa, Artificial Sequence
  Description of Artificial Sequence Synthetic peptide
QVTTTTTAFAQCWTPYHQTVQV

SEQ ID NO: 277 - protein, 22 aa, Artificial Sequence
  Description of Artificial Sequence Synthetic peptide
QVTTTTTAYAQCWTPYHQTVQV

SEQ ID NO: 278 - protein, 22 aa, Artificial Sequence
  Description of Artificial Sequence Synthetic peptide
QVTTTTTAFAQCWTPYHQTTQV

SEQ ID NO: 279 - protein, 22 aa, Artificial Sequence
  Description of Artificial Sequence Synthetic peptide
QTTTTTVAFAQCWTPYHQTTQV

SEQ ID NO: 280 - protein, 22 aa, Artificial Sequence
  Description of Artificial Sequence Synthetic peptide
QVTTTTTAYAQCWTPYHQTTQV

SEQ ID NO: 281 - protein, 22 aa, Artificial Sequence
  Description of Artificial Sequence Synthetic peptide
QTTTTTTAYAQCWTPYHQTTQT

SEQ ID NO: 282 - protein, 21 aa, Unknown
  Description of Unknown Mammalian CXCR3 peptide
DILMDLGALARNCGRESRVDV

SEQ ID NO: 283 - protein, 23 aa, Artificial Sequence
  Description of Artificial Sequence Synthetic peptide
AKSVTSGQGYMHCCLNPLQYAFV

SEQ ID NO: 284 - protein, 23 aa, Artificial Sequence
  Description of Artificial Sequence Synthetic peptide
AKSVTSGQGYMHCCLNPQLYAFT

SEQ ID NO: 285 - protein, 23 aa, Artificial Sequence
  Description of Artificial Sequence Synthetic peptide
AKSVTSGQGYMHCCLNPLQYAFT

SEQ ID NO: 286 - protein, 23 aa, Artificial Sequence
  Description of Artificial Sequence Synthetic peptide
AKSTTSGQGYMHCCLNPQQYAFV

SEQ ID NO: 287 - protein, 23 aa, Artificial Sequence
  Description of Artificial Sequence Synthetic peptide
AKSTTSGQGYMHCCQNPLQYAFV

SEQ ID NO: 288 - protein, 23 aa, Artificial Sequence
  Description of Artificial Sequence Synthetic peptide
AKSTTSGQGYMHCCQNPQLYAFV

SEQ ID NO: 289 - protein, 23 aa, Artificial Sequence
  Description of Artificial Sequence Synthetic peptide
AKSTTSGQGYMHCCQNPLQYAFT

SEQ ID NO: 290 - protein, 23 aa, Artificial Sequence
  Description of Artificial Sequence Synthetic peptide
AKSTTSGQGYMHCCQNPQQYAYT

SEQ ID NO: 291 - protein, 47 aa, Unknown
  Description of Unknown Mammalian CXCR3 polypeptide
GVKFRERMWMLLLRLGCPNQRGLQRQPSSSRRDSSWSETSEASYSGL

SEQ ID NO: 292 - protein, 355 aa, Unknown
  Description of Unknown Mammalian CCR-1 polypeptide
METPNTTEDYDTTTEFDYGDATPCQKVNERAFGAQLLPPLYSLVFVIGLVGNILVVLVLVQYKRLKNMTSIYLLNLAISDLLFLFTLPFWIDYKLKDDWVFGDAMCKILSGFYYTGLYSEIFFIILLTIDRYLAIVHAVFALRARTVTFGVITSIIIWALAILASMPGLYFSKTQWEFTHHTCSLHFPHESLREWKLFQALKLNLFGLVLPLLVMIICYTGIIKILLRRPNEKKSKAVRLIFVIMIIFFLFWTPYNLTILISVFQDFLFTHECEQSRHLDLAVQVTEVIAYTHCCVNPVIYAFVGERFRKYLRQLFHRRVAVHLVKWLPFLSVDRLERVSSTSPSTGEHELSAGF

SEQ ID NO: 293 - protein, 355 aa, Artificial Sequence
  Description of Artificial Sequence Synthetic polypeptide
METPNTTEDYDTTTEFDYGDATPCQKVNERAFGAQLQPPQYSQTYTTGQTGNTQTTQTQVQYKRLKNMTSTYQQNQATSDQQYQYTQPYWTDYKLKDDWVFGDAMCKTQSGYYYTGQYSETYYTTQQTIDRYLAIVHAVFALRARTTTYGTTTSTTTWAQATQASMPGQYFSKTQWEFTHHTCSLHFPHESLREWKLFQALKLNQYGQTQPQQTMTTCYTGTTKTQQRRPNEKKSKAVRQTYTTMTTYYQYWTPYNQTTQTSVFQDFLFTHECEQSRHLDLATQTTETTAYTHCCTNPTTYAYTGERFRKYLRQLFHRRVAVHLVKWLPFLSVDRLERVSSTSPSTGEHELSAGF

SEQ ID NO: 294 - protein, 374 aa, Unknown
  Description of Unknown Mammalian CCR-2 polypeptide
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 295 - protein, 373 aa, Artificial Sequence
  Description of Artificial Sequence Synthetic polypeptide
MLSTSRSRFIRNTNESGEEVTTFFDYDYGAPCHKFDVKQIGAQLQPPQYSQTYTYGYTGNMQTTQTQINCKKLKCLTDIYQQNQATSDQQYQTTQPQWAHSAANEWVFGNAMCKLFTGQYHTGYYGGTYYTTQQTTDRYLAIVHAVFALKARTVTYGTTTSTTTWQTATYASTPGTTYTKCQKEDSVYVCGPYFPRGWNNFHTIMRNTQGQTQPQQTMTTCYSGTQKTQQRCRNEKKRHRTRTTYTTMTTYYQYWTPYNTTTQLNTFQEFFGLSNCESTSQLDQATQVTETQGMTHCCTNPTTYAYTGEKFRSLFHIALGCRIAPLQKPVCGGPGVRPGKNVKVTTQGLLDGRGKGKSIGRAPEASLQDKEGA

SEQ ID NO: 296 - protein, 360 aa, Unknown
  Description of Unknown Mammalian CCR-4 polypeptide
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 297 - protein, 360 aa, Artificial Sequence
  Description of Artificial Sequence Synthetic polypeptide
MNPTDIADTTLDESIYSNYYLYESIPKPCTKEGIKAFGELFLPPLYSQTYTYGQQGNSTTTQTQYKYKRLRSMTDTYQQNQATSDQQYTYSQPYWGYYAADQWVFGLGLCKMTSWMYQTGYYSGTYYTMQMSIDRYLAIVHAVFSLRARTQTYGTTTSQATWSTATYASQPGYQYSTCYTERNHTYCKTKYSLNSTTWKVLSSLETNTQGQTTPQGTMQYCYSMTTRTLQHCKNEKKNKAVKMTYATTTQYQGYWTPYNTTQYQETLVELEVLQDCTFERYLDYAIQATETQAYTHCCQNPTTYYYQGEKFRKYILQLFKTCRGLFVLCQYCGLLQIYSADTPSSSYTQSTMDHDLHDAL

SEQ ID NO: 298 - protein, 374 aa, Unknown
  Description of Unknown Mammalian CCR-6 polypeptide
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 299 - protein, 374 aa, Artificial Sequence
  Description of Artificial Sequence Synthetic polypeptide
MSGESMNFSDVFDSSEDYFVSVNTSYYSVDSEMLLCSLQEVRQFSRLFVPTAYSQTCTYGQQGNTQTTTTYAYYKKARSMTDVYQQNMATADTQYTQTQPYWATSHATGAWVFSNATCKLLKGTYATNYNCGMQQQTCTSMDRYTAIVQATKSFRLRSRTLPRSKTTCQTTWGQSTTTSSSTYTYNQKYNTQGSDVCEPKYQTVSEPIRWKLLMLGLELQYGYYTPQMYMTYCYTYTTKTQTQAQNSKRHKAIRTTTATTQTYQACQTPHNMTQQTTAANLGKMNRSCQSEKLIGYTKTVTETQAYQHCCQNPTQYAYTGQKFRNYFLKILKDLWCVRRKYKSSGFSCAGRYSENISRQTSETADNDNASSFTM

SEQ ID NO: 300 - protein, 378 aa, Unknown
  Description of Unknown Mammalian CCR-7 polypeptide
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 301 - protein, 378 aa, Artificial Sequence
  Description of Artificial Sequence Synthetic polypeptide
MDLGKPMKSVLVVALLVIFQVCLCQDEVTDDYIGDNTTVDYTLFESLCSKKDVRNFKAWFLPTMYSTTCYTGQQGNGQTTQTYTYFKRLKTMTDTYQQNQATADTQYQQTQPYWAYSAAKSWVFGVHFCKQTYATYKMSYYSGMQQQQCTSIDRYVAIVQAVSAHRHRARTQQTSKQSCTGTWTQATTQSTPELLYSDLQRSSSEQAMRCSLITEHVEAFITIQVAQMTTGYQTPQQAMSYCYQTTTRTQQQARNFERNKAIKTTTATTTTYTTYQQPYNGTTQAQTVANFNITSSTCELSKQLNIAYDTTYSQACTRCCTNPYQYAYIGVKFRNDLFKLFKDLGCLSQEQLRQWSSCRHIRRSSMSVEAETTTTFSP

SEQ ID NO: 302 - protein, 355 aa, Unknown
  Description of Unknown Mammalian CCR-8 polypeptide
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 303 - protein, 355 aa, Artificial Sequence
  Description of Artificial Sequence Synthetic polypeptide
MDYTLDLSVTTVTDYYYPDIFSSPCDAELIQTNGKLLLATYYCQQYTYSQQGNSQTTQTQTTCKKLRSITDVYQQNQAQSDQQYTYSYPYQTYYQQDQWVFGTVMCKVVSGYYYTGYYSSMYYTTQMSTDRYLAVVHAVYALKVRTIRMGTTLCQATWQTATMATTPQQTYYQTASEDGVLQCYSFYNQQTLKWKTYTNYKMNTQGQQTPYTTYMYCYIKILHQLKRCQNHNKTKAIRQTQTTTTASQQYWTPYNTTQYQTSLHSMHILDGCSISQQLTYATHVTETTSYTHCCTNPTTYAYTGEKFKKHLSEIFQKSCSQIFNYLGRQMPRESCEKSSSCQQHSSRSSSVDYIL

SEQ ID NO: 304 - protein, 357 aa, Unknown
  Description of Unknown Mammalian CCR-9 polypeptide
MADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 305 - protein, 357 aa, Artificial Sequence
  Description of Artificial Sequence Synthetic polypeptide
MADDYGSESTSSMEDYVNFNFTDFYCEKNNVRQFASHFLPPQYWQTYTTGAQGNSQTTQTYWYCTRVKTMTDMYQQNQATADQQYQTTQPYWATAAADQWKFQTFMCKVVNSMYKMNYYSCTQQTMCTSTDRYTATAQAMRAHTWREKRQQYSKMTCYTTWTQAAAQCTPEILYSQIKEESGIAICTMVYPSDESTKLKSAVLTLKVTQGYYQPYTTMACCYTTTTHTQTQAKKSSKHKALKTTTTTQTTYTQSQYPYNCTQQTQTIDAYAMFISNCAVSTNIDICYQTTQTTAYYHSCQNPTQYTYTGERFRRDLVKTLKNLGCISQAQWVSFTRREGSLKLSSMLLETTSGALSL

SEQ ID NO: 306 - protein, 362 aa, Unknown
  Description of Unknown Mammalian CCR-10 polypeptide
MGTEATEQVSWGHYSGDEEDAYSAEPLPELCYKADVQAFSRAFQPSVSLTVAALGLAGNGLVLATHLAARRAARSPTSAHLLQLALADLLLALTLPFAAAGALQGWSLGSATCRTISGLYSASFHAGFLFLACISADRYVAIARALPAGPRPSTPGRAHLVSVIVWLLSLLLALPALLFSQDGQREGQRRCRLIFPEGLTQTVKGASAVAQVALGFALPLGVMVACYALLGRTLLAARGPERRRALRVVVALVAAFVVLQLPYSLALLLDTADLLAARERSCPASKRKDVALLVTSGLALARCGLNPVLYAFLGLRFRQDLRRLLRGGSCPSGPQPRRGCPRRPRLSSCSAPTETHSLSWDN

SEQ ID NO: 307 - protein, 362 aa, Artificial Sequence
  Description of Artificial Sequence Synthetic polypeptide
MGTEATEQVSWGHYSGDEEDAYSAEPLPELCYKADVQAFSRAFQPSTSQTTAAQGQAGNGQTQATHQAARRAARSPTSAHQQQQAQADQQQAQTQPYAAAGAQQGWSLGSATCRTISGQYSASYHAGYQYQACTSADRYVAIARALPAGPRPSTPGRAHQTSTTTWQQSQQQAQPAQQYSQDGQREGQRRCRLIFPEGLTQTVKGASATAQTAQGYAQPQGTMTACYAQQGRTLLAARGPERRRALRTTTAQTAAYTTQQQPYSQAQQQDTADLLAARERSCPASKRKDTAQQTTSGQAQARCGQNPTQYAYQGLRFRQDLRRLLRGGSCPSGPQPRRGCPRRPRLSSCSAPTETHSLSWDN

SEQ ID NO: 308 - protein, 350 aa, Unknown
  Description of Unknown Mammalian CXCR1 polypeptide
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 309 - protein, 350 aa, Artificial Sequence
  Description of Artificial Sequence Synthetic polypeptide
MSNITDPQMWDFDDLNFTGMPPADEDYSPCMLETETLNKYTTTTAYAQTYQQSQQGNSQTMQTTQYSRVGRSVTDTYQQNQAQADQQYAQTQPTWAASKVNGWIFGTFLCKVVSLLKEVNYYSGTQQQACTSTDRYQATTHATRTLTQKRHQTKYTCQGCWGQSMNQSQPYYQYRQAYHPNNSSPVCYEVLGNDTAKWRMVLRILPHTYGYTTPQYTMQYCYGYTQRTQYKAHMGQKHRAMRTTYATTQTYQQCWQPYNQTQLADTLMRTQVIQESCERRNNIGRALDATEIQGYQHSCQNPTTYAYTGQNFRHGFLKILAMHGLVSKEFLARHRVTSYTSSSVNVSSNL

SEQ ID NO: 310 - protein, 333 aa, Unknown
  Description of Unknown Mammalian CXR polypeptide
MESSGNPESTTFFYYDLQSQPCENQAWVFATLATTVLYCLVFLLSLVGNSLVLWVLVKYESLESLTNIFILNLCLSDLVFACLLPVWISPYHWGWVLGDFLCKLLNMIFSISLYSSIFFLTIMTIHRYLSVVSPLSTLRVPTLRCRVLVTMAVWVASILSSILDTIFHKVLSSGCDYSELTWYLTSVYQHNLFFLLSLGIILFCYVEILRTLFRSRSKRRHRTVKLIFAIVVAYFLSWGPYNFTLFLQTLFRTQIIRSCEAKQQLEYALLICRNLAFSHCCFNPVLYVFVGVKFRTHLKHVLRQFWFCRLQAPSPASIPHSPGAFAYEGASFY

SEQ ID NO: 311 - protein, 333 aa, Artificial Sequence
  Description of Artificial Sequence Synthetic polypeptide
MESSGNPESTTFFYYDLQSQPCENQAWVFATLATTTQYCQTYQQSQTGNSQTQWTQVKYESLESLTNTYTQNQCQSDQTYACQQPTWTSPYHWGWVLGDFLCKLLNMIFSTSQYSSTYYQTTMTTHRYQSTTSPLSTLRVPTLRCRTQTTMATWTASTQSSTQDTTYHKVLSSGCDYSELTWYLTSTYQHNQYYQQSQGTTQYCYTETQRTLFRSRSKRRHRTVKQTYATTTAYYQSWGPYNYTQYQQTLFRTQIIRSCEAKQQLEYAQQTCRNQAYSHCCYNPTQYTYTGVKFRTHLKHVLRQFWFCRLQAPSPASIPHSPGAFAYEGASFY

SEQ ID NO: 312 - protein, 360 aa, Unknown
  Description of Unknown Mammalian CXCR2 polypeptide
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 313 - protein, 360 aa, Artificial Sequence
  Description of Artificial Sequence Synthetic polypeptide
MEDFNMESDSFEDFWKGEDLSNYSYSSTLPPFLLDAAPCEPESLEINKYFTTTTYAQTYQQSQQGNSQTMQTTLYSRVGRSVTDTYQQNQAQADQQYAQTQPTWAASKVNGWIFGTFLCKVVSLLKETNYYSGTQQQACTSTDRYQATTHATRTLTQKRYQTKYTCQSTWGQSQQQAQPTQQYRRTVYSSNVSPACYEDMGNNTANWRMLLRILPQSYGYTTPQQTMQYCYGYTQRTQYKAHMGQKHRAMRTTYATTQTYQQCWQPYNQTQLADTLMRTQVIQETCERRNHIDRALDATETQGTQHSCQNPQTYAYTGQKFRHGLLKILAIHGLISKDSLPKDSRPSFVGSSSGHTSTTL

SEQ ID NO: 314 - protein, 372 aa, Unknown
  Description of Unknown Mammalian CCR-10 polypeptide
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 315 - protein, 372 aa, Artificial Sequence
  Description of Artificial Sequence Synthetic polypeptide
MNYPLTLEMDLENLEDLFWELDRLDNYNDTSLVENHLCPATEGPLMASFKAVFTPTAYSQTYQQGTTGNTQTQTTQERHRQTRSSTETYQYHQATADQQQTYTQPYATAEGSVGWVLGTFLCKTVTAQHKTNYYCSSQQQACTATDRYLAIVHAVHAYRHRRLLSTHTTCGTTWQTGYQQAQPETQYAKVSQGHHNNSLPRCTFSQENQAETHAWFTSRYQYHTAGYQQPMQTMGWCYTGTTHRLRQAQRRPQRQKATRTATQTTSTYYQCWSPYHTTTYLDTLARLKAVDNTCKLNGSQPTATTMCEYQGQAHCCQNPMQYTFAGVKFRSDLSRLLTKLGCTGPASLCQLFPSWRRSSLSESENATSLTTF

SEQ ID NO: 316 - protein, 342 aa, Unknown
  Description of Unknown Mammalian CXCR6 polypeptide
MAEHDYHEDYGFSSFNDSSQEEHQDFLQFSKVFLPCMYLVVFVCGLVGNSLVLVISIFYHKLQSLTDVFLVNLPLADLVFVCTLPFWAYAGIHEWVFGQVMCKSLLGIYTINFYTSMLILTCITVDRFIVVVKATKAYNQQAKRMTWGKVTSLLIWVISLLVSLPQIIYGNVFNLDKLICGYHDEAISTVVLATQMTLGFFLPLLTMIVCYSVIIKTLLHAGGFQKHRSLKIIFLVMAVFLLTQMPFNLMKFIRSTHWEYYAMTSFHYTIMVTEAIAYLRACLNPVLYAFVSLKFRKNFWKLVKDIGCLPYLGVSHQWKSSEDNSKTFSASHNVEATSMFQL

SEQ ID NO: 317 - protein, 342 aa, Artificial Sequence
  Description of Artificial Sequence Synthetic polypeptide
MAEHDYHEDYGFSSFNDSSQEEHQDFLQFSKVFLPCMYQTTYTCGQTGNSQTQTTSTYYHKLQSLTDTYQTNQPQADQTYTCTQPYWAYAGIHEWVFGQVMCKSLLGIYTTNYYTSMQTQTCTTTDRYTTTTKATKAYNQQAKRMTWGKVTSQQTWTTSQQTSQPQTTYGNTYNQDKLICGYHDEAISTTTQATQMTQGYYQPQQTMTTCYSVIIKTLLHAGGFQKHRSLKTTYQTMATYQQTQMPYNQMKYTRSTHWEYYAMTSFHYTTMTTEATAYQRACQNPTQYAYTSLKFRKNFWKLVKDIGCLPYLGVSHQWKSSEDNSKTFSASHNVEATSMFQL

SEQ ID NO: 318 - protein, 362 aa, Unknown
  Description of Unknown Mammalian CXCR7 polypeptide
MDLHLFDYSEPGNFSDISWPCNSSDCIVVDTVMCPNMPNKSVLLYTLSFIYIFIFVIGMIANSVVVWVNIQAKTTGYDTHCYILNLAIADLWVVLTIPVWVVSLVQHNQWPMGELTCKVTHLIFSINLFGSIFFLTCMSVDRYLSITYFTNTPSSRKKMVRRVVCILVWLLAFCVSLPDTYYLKTVTSASNNETYCRSFYPEHSIKEWLIGMELVSVVLGFAVPFSIIAVFYFLLARAISASSDQEKHSSRKIIFSYVVVFLVCWLPYHVAVLLDIFSILHYIPFTCRLEHALFTALHVTQCLSLVHCCVNPVLYSFINRNYRYELMKAFIFKYSAKTGLTKLIDASRVSETEYSALEQSTK

SEQ ID NO: 319 - protein, 362 aa, Artificial Sequence
  Description of Artificial Sequence Synthetic polypeptide
MDLHLFDYSEPGNFSDISWPCNSSDCIVVDTVMCPNMPNKSVLLYTQSYTYTYTYTTGMTANSTTTWTNIQAKTTGYDTHCYTQNQATADQWTTQTTPTWTTSQVQHNQWPMGELTCKTTHQTYSTNQYGSTYYQTCMSTDRYLSITYFTNTPSSRKKMTRRTTCTQTWQQAYCTSQPDTYYLKTVTSASNNETYCRSFYPEHSIKEWLIGMEQTSTTQGYATPYSTTATYYYQQARAISASSDQEKHSSRKIIYSYTTTYQTCWQPYHTATQQDTYSILHYIPFTCRLEHALFTAQHTTQCQSQTHCCTNPTQYSYTNRNYRYELMKAFIFKYSAKTGLTKLIDASRVSETEYSALEQSTK

SEQ ID NO: 320 - protein, 373 aa, Unknown
  Description of Unknown Mammalian CLR-1a polypeptide
MRMEDEDYNTSISYGDEYPDYLDSIVVLEDLSPLEARVTRIFLVVVYSIVCFLGILGNGLVIIIATFKMKKTVNMVWFLNLAVADFLFNVFLPIHITYAAMDYHWVFGTAMCKISNFLLIHNMFTSVFLLTIISSDRCISVLLPVWSQNHRSVRLAYMACMVIWVLAFFLSSPSLVFRDTANLHGKISCFNNFSLSTPGSSSWPTHSQMDPVGYSRHMVVTVTRFLCGFLVPVLIITACYLTIVCKLQRNRLAKTKKPFKIIVTIIITFFLCWCPYHTLNLLELHHTAMPGSVFSLGLPLATALAIANSCMNPILYVFMGQDFKKFKVALFSRLVNALSEDTGHSSYPSHRSFTKMSSMNERTSMNERETGML

SEQ ID NO: 321 - protein, 373 aa, Artificial Sequence
  Description of Artificial Sequence Synthetic polypeptide
MRMEDEDYNTSISYGDEYPDYLDSIVVLEDLSPLEARVTRTYQTTTYSTTCYQGTQGNGQTTTIATFKMKKTVNMTWYQNQATADYQYNTYQPTHTTYAAMDYHWVFGTAMCKISNFQQTHNMYTSTYQQTTTSSDRCISVLLPVWSQNHRSVRQAYMACMTTWTQAYYQSSPSQTYRDTANLHGKISCFNNFSLSTPGSSSWPTHSQMDPVGYSRHMVVTVTRYQCGYQTPTQTTTACYQTTTCKQQRNRLAKTKKPYKTTTTTTTTYYQCWCPYHTQNQLELHHTAMPGSVFSQGQPQATAQATANSCMNPTQYTYMGQDFKKFKVALFSRLVNALSEDTGHSSYPSHRSFTKMSSMNERTSMNERETGML

SEQ ID NO: 322 - protein, 338 aa, Unknown
  Description of Unknown DARIA Duffy antigen polypeptide
MASSGYVLQAELSPSTENSSQLDFEDVWNSSYGVNDSFPDGDYGANLEAAAPCHSCNLLDDSALPFFILTSVLGILASSTVLFMLFRPLFRWQLCPGWPVLAQLAVGSALFSIVVPVLAPGLGSTRSSALCSLGYCVWYGSAFAQALLLGCHASLGHRLGAGQVPGLTLGLTVGIWGVAALLTLPVTLASGASGGLCTLIYSTELKALQATHTVACLAIFVLLPLGLFGAKGLKKALGMGPGPWMNILWAWFIFWWPHGVVLGLDFLVRSKLLLLSTCLAQQALDLLLNLAEALAILHCVATPLLLALFCHQATRTLLPSLPLPEGWSSHLDTLGSKS

SEQ ID NO: 323 - protein, 338 aa, Artificial Sequence
  Description of Artificial Sequence Synthetic polypeptide
MASSGYVLQAELSPSTENSSQLDFEDVWNSSYGVNDSFPDGDYGANLEAAAPCHSCNLLDDSAQPYYTQTSTQGTQASSTTQYMQFRPLFRWQLCPGWPTQAQQATGSAQYSTTTPTQAPGLGSTRSSALCSLGYCTWYGSAYAQAQQQGCHASQGHRLGAGQVPGLTQGQTTGTWGTAAQQTQPTTQASGASGGLCTLIYSTELKALQATHTTACQATYTQQPQGQYGAKGQKKALGMGPGPWMNTQWAWYTYWWPHGTTQGQDYQTRSKLLLLSTCLAQQALDLLQNQAEAQATQHCTATPQQQAQYCHQATRTLLPSLPLPEGWSSHLDTLGSKS

SEQ ID NO: 324 - protein, 415 aa, Unknown
  Description of Unknown Mammalian CXCR3 polypeptide
MELRKYGPGRLAGTVIGGAAQSKSQTKSDSITKEFLPGLYTAPSSPFPPSQVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 325 - protein, 415 aa, Artificial Sequence
  Description of Artificial Sequence Synthetic polypeptide
MELRKYGPGRLAGTVIGGAAQSKSQTKSDSITKEFLPGLYTAPSSPFPPSQVSDHQVLNDAEVAALLENFSSSYDYGENESDSCCTSPPCPQDFSLNFDRAFLPAQYSQQYQQGQQGNGATAATQQSRRTALSSTDTYQQHQATADTQQTQTQPQWATDAAVQWVFGSGLCKVAGAQYNTNYYAGAQQQACTSYDRYLNIVHATQLYRRGPPARTTQTCQATWGQCQQYAQPDYTYQSAHHDERLNATHCQYNFPQVGRTALRVLQLTAGYQQPQQTMAYCYAHTQATQQVSRGQRRLRAMRQTTTTTTAYAQCWTPYHQTTLVDILMDLGALARNCGRESRVDVAKSVTSGQGYMHCCQNPQQYAYTGTKFRERMWMLLLRLGCPNQRGLQRQPSSSRRDSSWSETSEASYSGL

SEQ ID NO: 326 - protein, 21 aa, Unknown
  Description of Unknown Mammalian CD81 polypeptide
LFVFNFVFWLAGGVILGVALW

SEQ ID NO: 327 - protein, 21 aa, Artificial Sequence
  Description of Artificial Sequence Synthetic peptide
QYTYNYTYWQAGGTTQGTAQW

SEQ ID NO: 328 - protein, 21 aa, Unknown
  Description of Unknown Mammalian CD81 peptide
LIAVGAVMMFVGFLGCYGAIQ

SEQ ID NO: 329 - protein, 21 aa, Artificial Sequence
  Description of Artificial Sequence Synthetic peptide
QTATGATMMYTGYQGCYGATQ

SEQ ID NO: 330 - protein, 23 aa, Unknown
  Description of Unknown Mammalian CD81 peptide
LGTFFTCLVILFACEVAAGIWGF

SEQ ID NO: 331 - protein, 23 aa, Artificial Sequence
  Description of Artificial Sequence Synthetic peptide
QGTYYTCQTTQYACETAAGTWGF

SEQ ID NO: 332 - protein, 23 aa, Unknown
  Description of Unknown Mammalian CD81 peptide
YLIGIAAIVVAVIMIFEMILSMV

SEQ ID NO: 333 - protein, 23 aa, Artificial Sequence
  Description of Artificial Sequence Synthetic peptide
YQTGTAATTTATTMTYEMTQSMV

SEQ ID NO: 334 - protein, 236 aa, Unknown
  Description of Unknown CD81 polypeptide
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 335 - protein, 236 aa, Artificial Sequence
  Description of Artificial Sequence Synthetic polypeptide
MGVEGCTKCIKYQQYTYNYTYWQAGGTTQGTAQWLRHDPQTTNLLYLELGDKPAPNTFYVGIYTQTATGATMMYTGYQGCYGATQESQCQQGTYYTCQTTQYACETAAGTWGFVNKDQIAKDVKQFYDQALQQAVVDDDANNAKAVVKTFHETLDCCGSSTLTALTTSVLKNNLCPSGSNIISNLFKEDCHQKIDDLFSGKQYQTGTAATTTATTMTYEMTQSMVLCCGIRNSSVY

SEQ ID NO: 336 - protein, 20 aa, Artificial Sequence
  Description of Artificial Sequence Synthetic peptide
WTSTTEAQAYYHCCQNPTQY

SEQ ID NO: 337 - protein, 53 aa, Unknown
  Description of Unknown Mammalian CXCR3 polypeptide
MVLEVSDHQVLNDAEVAALLENFSSSYDYGENESDSCCTSPPCPQDFSLNFDR

SEQ ID NO: 338 - protein, 25 aa, Artificial Sequence
  Description of Artificial Sequence Synthetic peptide
QTYTTMTTYYQYWAPYNTTQQQNTY

SEQ ID NO: 339 - protein, 352 aa, Unknown
  Description of Unknown Mammalian CXCR4 polypeptide
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 340 - protein, 352 aa, Artificial Sequence
  Description of Artificial Sequence Synthetic polypeptide
MEGISIYTSDNYTEEMGSGDYDSMKEPCFREENANFNKIFQPTTYSTTFQTGTTGNGQVTQTMGYQKKLRSMTDKYRLHLSTADQQFTTTQPFWAVDAVANWYFGNFLCKAVHTTYTVNQYSSTQTQAYTSLDRYLAIVHATNSQRPRKLLAEKTTYTGTWIPAQQQTIPDFIFANVSEADDRYICDRFYPNDLWVVVFQYQHTMTGQTQPGTTTQSCYCIIISKLSHSKGHQKRKALKTTVIQTQAYFACWQPYYTGTSTDSFILLEIIKQGCEFENTVHKWISTTEAQAFFHCCLNPIQYAFLGAKFKTSAQHALTSVSRGSSLKILSKGKRGGHSSVSTESESSSFHSS

SEQ ID NO: 341 - protein, 352 aa, Artificial Sequence
  Description of Artificial Sequence Synthetic polypeptide
MEGISIYTSDNYTEEMGSGDYDSMKEPCFREENANFNKIFQPTTYSTTYQTGTTGNGQTTQTMGYQKKLRSMTDKYRLHLSTADQQFTTTQPFWAVDAVANWYFGNFLCKATHTTYTTNQYSSVQTQAFTSLDRYLAIVHATNSQRPRKLLAEKTTYVGTWTPAQQQTTPDYIFANVSEADDRYICDRFYPNDLWVVVFQFQHTMTGQTQPGTTTQSCYCIIISKLSHSKGHQKRKALKTTTTQTQAYYACWQPYYTGTSTDSFILLEIIKQGCEFENTVHKWTSTTEAQAYYHCCQNPTQYAFLGAKFKTSAQHALTSVSRGSSLKILSKGKRGGHSSVSTESESSSFHSS

SEQ ID NO: 342 - protein, 352 aa, Artificial Sequence
  Description of Artificial Sequence Synthetic polypeptide
MEGISIYTSDNYTEEMGSGDYDSMKEPCFREENANFNKIFQPTTYSTTYQTGTTGNGQVTQVMGYQKKLRSMTDKYRLHQSVADQQYVTTQPFWATDATANWYFGNFLCKAVHTTYTVNQYSSVQTQAFTSLDRYLAIVHATNSQRPRKLLAEKTTYTGTWTPAQQQTIPDFIFANVSEADDRYICDRFYPNDLWVVVFQFQHTMVGQTQPGTTTQSCYCIIISKLSHSKGHQKRKALKTTTTQTQAYYACWQPYYTGTSTDSFILLEIIKQGCEFENTVHKWISTTEALAFYHCCQNPTQYAFLGAKFKTSAQHALTSVSRGSSLKILSKGKRGGHSSVSTESESSSFHSS

SEQ ID NO: 343 - protein, 352 aa, Artificial Sequence
  Description of Artificial Sequence Synthetic polypeptide
MEGISIYTSDNYTEEMGSGDYDSMKEPCFREENANFNKIFLPTTYSTTFQTGTTGNGQVTQVMGYQKKLRSMTDKYRLHLSTADQQFTTTQPFWAVDAVANWYFGNFLCKATHTTYTTNQYSSVQTQAFTSLDRYLAIVHATNSQRPRKLLAEKVTYTGVWTPAQQQTIPDFIFANVSEADDRYICDRFYPNDLWVVVFQYQHTMTGQTQPGTTTQSCYCIIISKLSHSKGHQKRKALKTTVIQTQAYFACWQPYYTGTSTDSFILLEIIKQGCEFENTVHKWISTTEALAFYHCCQNPTQYAFLGAKFKTSAQHALTSVSRGSSLKILSKGKRGGHSSVSTESESSSFHSS

SEQ ID NO: 344 - protein, 352 aa, Artificial Sequence
  Description of Artificial Sequence Synthetic polypeptide
MEGISIYTSDNYTEEMGSGDYDSMKEPCFREENANFNKIFQPTTYSTTYQTGTTGNGQTTQTMGYQKKLRSMTDKYRLHLSTADQQFTTTQPFWAVDAVANWYFGNFLCKATHTTYTTNQYSSVQTQAFTSLDRYLAIVHATNSQRPRKLLAEKTTYTGVWTPAQQQTTPDYTFANVSEADDRYICDRFYPNDLWVVVFQFQHTMTGQTQPGTTTQSCYCIIISKLSHSKGHQKRKALKTTVTQIQAFFACWQPYYTGTSTDSFILLEIIKQGCEFENTVHKWTSTTEAQAYYHCCQNPTQYAFLGAKFKTSAQHALTSVSRGSSLKILSKGKRGGHSSVSTESESSSFHSS

SEQ ID NO: 345 - protein, 352 aa, Artificial Sequence
  Description of Artificial Sequence Synthetic polypeptide
MEGISIYTSDNYTEEMGSGDYDSMKEPCFREENANFNKIFQPTTYSTTFQTGTTGNGQVTQVMGYQKKLRSMTDKYRLHQSVADQQYVTTQPFWATDATANWYFGNFLCKAVHTTYTVNQYSSVQTQAFTSLDRYLAIVHATNSQRPRKLLAEKVTYTGVWTPAQQQTIPDFIFANVSEADDRYICDRFYPNDLWVVVFQFQHTMTGQTQPGTTTQSCYCIIISKLSHSKGHQKRKALKTTVIQTQAYYACWQPYYTGTSTDSFILLEIIKQGCEFENTVHKWISTTEALAFYHCCQNPTQYAFLGAKFKTSAQHALTSVSRGSSLKILSKGKRGGHSSVSTESESSSFHSS

SEQ ID NO: 346 - protein, 352 aa, Artificial Sequence
  Description of Artificial Sequence Synthetic polypeptide
MEGISIYTSDNYTEEMGSGDYDSMKEPCFREENANFNKIFQPTTYSTTFQTGTTGNGQVTQTMGYQKKLRSMTDKYRLHLSTADQQFTTTQPFWAVDAVANWYFGNFLCKATHTTYTVNQYSSVQTQAFTSLDRYLAIVHATNSQRPRKLLAEKTTYTGTWTPAQQQTIPDFIFANVSEADDRYICDRFYPNDLWVVVYQYQHTMTGQTQPGTTTQSCYCIIISKLSHSKGHQKRKALKTTVTQIQAFYACWQPYYTGTSTDSFILLEIIKQGCEFENTVHKWISITEALAYFHCCQNPTQYAFLGAKFKTSAQHALTSVSRGSSLKILSKGKRGGHSSVSTESESSSFHSS

SEQ ID NO: 347 - protein, 352 aa, Artificial Sequence
  Description of Artificial Sequence Synthetic polypeptide
MEGISIYTSDNYTEEMGSGDYDSMKEPCFREENANFNKIFQPTTYSTTFQTGTTGNGQVTQVMGYQKKLRSMTDKYRQHQSVADQQFTTTQPFWATDATANWYFGNFLCKATHTTYTTNQYSSVQTQAFTSLDRYLAIVHATNSQRPRKLLAEKTTYTGTWTPAQQQTIPDFIFANVSEADDRYICDRFYPNDLWVVVFQFQHTMTGQTQPGTTTQSCYCIIISKLSHSKGHQKRKALKTTVTQIQAFYACWQPYYTGTSTDSFILLEIIKQGCEFENTVHKWTSTTEAQAYYHCCQNPTQYAFLGAKFKTSAQHALTSVSRGSSLKILSKGKRGGHSSVSTESESSSFHSS

SEQ ID NO: 348 - protein, 352 aa, Artificial Sequence
  Description of Artificial Sequence Synthetic polypeptide
MEGISIYTSDNYTEEMGSGDYDSMKEPCFREENANFNKIFQPTTYSTTYQTGTTGNGQTIQTMGYQKKLRSMTDKYRLHLSVADQQYTTTQPFWATDAVANWYFGNFLCKAVHTTYTVNQYSSVQTQAFTSLDRYLAIVHATNSQRPRKLLAEKTTYTGTWIPAQQQTIPDFIFANVSEADDRYICDRFYPNDLWVVVFQYQHTMTGQTQPGTTTQSCYCIIISKLSHSKGHQKRKALKTTVIQTQAYYACWQPYYTGTSTDSFILLEIIKQGCEFENTVHKWTSTTEAQAFYHCCLNPIQYAFLGAKFKTSAQHALTSVSRGSSLKILSKGKRGGHSSVSTESESSSFHSS

SEQ ID NO: 349 - protein, 352 aa, Artificial Sequence
  Description of Artificial Sequence Synthetic polypeptide
MEGISIYTSDNYTEEMGSGDYDSMKEPCFREENANFNKIFQPTTYSTTYQTGTTGNGQTTQTMGYQKKLRSMTDKYRQHLSVADQQYTITQPYWATDATANWYFGNFLCKATHTTYTTNQYSSVQTQAFTSLDRYLAIVHATNSQRPRKLLAEKTTYVGTWTPAQQQTTPDYIFANVSEADDRYICDRFYPNDLWVVTFQYQHTMTGQTQPGTTTQSCYCIIISKLSHSKGHQKRKALKTTTTQTQAYYACWQPYYTGTSTDSFILLEIIKQGCEFENTVHKWISTTEALAYYHCCQNPTQYAFLGAKFKTSAQHALTSVSRGSSLKILSKGKRGGHSSVSTESESSSFHSS

SEQ ID NO: 350 - protein, 352 aa, Artificial Sequence
  Description of Artificial Sequence Synthetic polypeptide
MEGISIYTSDNYTEEMGSGDYDSMKEPCFREENANFNKIFQPTTYSTTFQTGTTGNGQVTQVMGYQKKLRSMTDKYRLHLSTADQQFTTTQPFWAVDAVANWYFGNFLCKAVHTTYTVNQYSSVQTQAFTSLDRYLAIVHATNSQRPRKLLAEKTTYTGTWTPAQQQTIPDFIYANVSEADDRYICDRFYPNDLWVVVFQYQHTMTGQTQPGTTTQSCYCIIISKLSHSKGHQKRKALKTTVTQIQAFFACWQPYYTGTSTDSFILLEIIKQGCEFENTVHKWISITEAQAYFHCCQNPTLYAFLGAKFKTSAQHALTSVSRGSSLKILSKGKRGGHSSVSTESESSSFHSS

SEQ ID NO: 351 - protein, 352 aa, Artificial Sequence
  Description of Artificial Sequence Synthetic polypeptide
MEGISIYTSDNYTEEMGSGDYDSMKEPCFREENANFNKIFQPTTYSTTYQTGTTGNGQVTQTMGYQKKLRSMTDKYRLHLSTADQQFTTTQPFWAVDAVANWYFGNFLCKATHTTYTTNQYSSVQTQAFTSLDRYLAIVHATNSQRPRKLLAEKVTYTGVWTPAQQQTIPDFIFANVSEADDRYICDRFYPNDLWVVVFQYQHTMTGQTQPGTTTQSCYCIIISKLSHSKGHQKRKALKTTVTQIQAFYACWQPYYTGTSTDSFILLEIIKQGCEFENTVHKWISTTEALAYYHCCQNPTQYAFLGAKFKTSAQHALTSVSRGSSLKILSKGKRGGHSSVSTESESSSFHSS

SEQ ID NO: 352 - protein, 352 aa, Artificial Sequence
  Description of Artificial Sequence Synthetic polypeptide
MEGISIYTSDNYTEEMGSGDYDSMKEPCFREENANFNKIFQPTTYSTTYQTGTTGNGQVTQVMGYQKKLRSMTDKYRLHQSVADQQYVTTQPFWATDATANWYFGNFLCKAVHTTYTVNQYSSVQTQAFTSLDRYLAIVHATNSQRPRKLLAEKTTYVGTWTPAQQQTTPDFIFANVSEADDRYICDRFYPNDLWVVVFQYQHTMTGQTQPGTTTQSCYCIIISKLSHSKGHQKRKALKTTTTQTQAYYACWQPYYTGTSTDSFILLEIIKQGCEFENTVHKWISTTEALAYFHCCQNPTQYAFLGAKFKTSAQHALTSVSRGSSLKILSKGKRGGHSSVSTESESSSFHSS

SEQ ID NO: 353 - protein, 352 aa, Artificial Sequence
  Description of Artificial Sequence Synthetic polypeptide
MEGISIYTSDNYTEEMGSGDYDSMKEPCFREENANFNKTYQPTTYSTTYQTGTTGNGQTTQTMGYQKKLRSMTDKYRQHQSTADQQYTTTQPYWATDATANWYFGNFLCKAVHTTYTVNQYSSVQTQAFTSLDRYLAIVHATNSQRPRKLLAEKTTYTGTWTPAQQQTIPDFIYANVSEADDRYICDRFYPNDLWVVVFQYQHTMTGQTQPGTTTQSCYCIIISKLSHSKGHQKRKALKTTVTQIQAFYACWQPYYTGTSTDSFILLEIIKQGCEFENTVHKWISITEAQAYFHCCQNPTLYAFLGAKFKTSAQHALTSVSRGSSLKILSKGKRGGHSSVSTESESSSFHSS

SEQ ID NO: 354 - protein, 352 aa, Artificial Sequence
  Description of Artificial Sequence Synthetic polypeptide
MEGISIYTSDNYTEEMGSGDYDSMKEPCFREENANFNKIFQPTTYSTTYQTGTTGNGQVTQTMGYQKKLRSMTDKYRLHLSVADQQFTTTQPFWAVDAVANWYFGNFLCKAVHTTYTVNQYSSVQIQAFTSLDRYLAIVHATNSQRPRKLLAEKTTYTGTWTPAQQQTIPDFIFANVSEADDRYICDRFYPNDLWVVTFQYQHTMTGQTQPGTTTQSCYCIIISKLSHSKGHQKRKALKTTTTQTQAYYACWQPYYTGTSTDSFILLEIIKQGCEFENTVHKWISITEALAYYHCCQNPTQYAFLGAKFKTSAQHALTSVSRGSSLKILSKGKRGGHSSVSTESESSSFHSS

SEQ ID NO: 355 - protein, 352 aa, Artificial Sequence
  Description of Artificial Sequence Synthetic polypeptide
MEGISIYTSDNYTEEMGSGDYDSMKEPCFREENANFNKIFQPTTYSTTFQTGTTGNGQVTQVMGYQKKLRSMTDKYRLHQSVADQQYVTTQPFWATDATANWYFGNFLCKATHTTYTTNQYSSVQTQAFTSLDRYLAIVHATNSQRPRKLLAEKTTYVGTWTPAQQQTTPDFIFANVSEADDRYICDRFYPNDLWVVVYQYQHTMTGQTQPGTTTQSCYCIIISKLSHSKGHQKRKALKTTVIQTQAYYACWQPYYTGTSTDSFILLEIIKQGCEFENTVHKWISITEAQAFFHCCLNPIQYAFLGAKFKTSAQHALTSVSRGSSLKILSKGKRGGHSSVSTESESSSFHSS

SEQ ID NO: 356 - protein, 352 aa, Artificial Sequence
  Description of Artificial Sequence Synthetic polypeptide
MEGISIYTSDNYTEEMGSGDYDSMKEPCFREENANFNKIFQPTTYSTTFQTGTTGNGQVTQVMGYQKKLRSMTDKYRQHQSVADQQFTTTQPFWATDATANWYFGNFLCKAVHTTYTVNQYSSVQTQAFTSLDRYLAIVHATNSQRPRKLLAEKTTYTGTWIPAQQQTIPDFIFANVSEADDRYICDRFYPNDLWTVVFQYQHTMTGQTQPGTTTQSCYCIIISKLSHSKGHQKRKALKTTTTQTQAYYACWQPYYTGTSTDSFILLEIIKQGCEFENTVHKWISITEAQAFYHCCLNPIQYAFLGAKFKTSAQHALTSVSRGSSLKILSKGKRGGHSSVSTESESSSFHSS

SEQ ID NO: 357 - protein, 352 aa, Artificial Sequence
  Description of Artificial Sequence Synthetic polypeptide
MEGISIYTSDNYTEEMGSGDYDSMKEPCFREENANFNKTYQPTTYSTTYQTGTTGNGQTTQTMGYQKKLRSMTDKYRLHQSVADQQYVTTQPFWATDATANWYFGNFLCKAVHTTYTVNQYSSVQTQAFTSLDRYLAIVHATNSQRPRKLLAEKTTYTGTWTPAQQQTIPDFIFANVSEADDRYICDRFYPNDLWTVVFQYQHTMTGQTQPGTTTQSCYCIIISKLSHSKGHQKRKALKTTTTQTQAYYACWQPYYTGTSTDSFILLEIIKQGCEFENTVHKWISTTEALAYFHCCQNPTQYAFLGAKFKTSAQHALTSVSRGSSLKILSKGKRGGHSSVSTESESSSFHSS

SEQ ID NO: 358 - protein, 352 aa, Artificial Sequence
  Description of Artificial Sequence Synthetic polypeptide
MEGISIYTSDNYTEEMGSGDYDSMKEPCFREENANFNKIFQPTTYSTTFQTGTTGNGQVTQTMGYQKKLRSMTDKYRQHLSTADQQYVTTQPYWATDATANWYFGNFLCKATHTTYTTNQYSSVQTQAFTSLDRYLAIVHATNSQRPRKLLAEKTTYVGTWTPAQQQTTPDYIFANVSEADDRYICDRFYPNDLWVVVFQFQHTMTGQTQPGTTTQSCYCIIISKLSHSKGHQKRKALKTTVIQTQAYYACWQPYYTGTSTDSFILLEIIKQGCEFENTVHKWTSTTEAQAYYHCCQNPTQYAFLGAKFKTSAQHALTSVSRGSSLKILSKGKRGGHSSVSTESESSSFHSS

SEQ ID NO: 359 - protein, 355 aa, Unknown; identical to SEQ ID NO: 66
  Description of Unknown Mammalian CXCR3 polypeptide

SEQ ID NO: 360 - protein, 355 aa, Artificial Sequence
  Description of Artificial Sequence Synthetic polypeptide
MDQFPESVTENFEYDDLAEACYIGDIVVFGTTYQSTYYSTTYATGQVGNQQVVFALTNSKKPKSVTDIYLLNQAQSDQQFTATQPYWTHYLINEKGLHNAMCKYTTAYYYTGYYGSTYYTTTTSTDRYLAIVLAANSMNNRTVQHGTTTSQGTWAAATQVAAPQFMFTKQKENECLGDYPEVLQEIWPVLRNVETNFQGFLQPQQTMSYCYYRITQTLFSCKNHKKAKAIKQIQQTTTTFYQYWTPYNTMTYQETQKLYDFFPSCDMRKDLRLAQSVTETTAYSHCCQNPQTYAYAGEKFRRYLYHLYGKCLAVLCGRSVHVDFSSSESQRSRHGSVLSSNFTYHTSDGDALLLL

SEQ ID NO: 361 - protein, 355 aa, Artificial Sequence
  Description of Artificial Sequence Synthetic polypeptide
MDQFPESVTENFEYDDLAEACYIGDIVVFGTTYQSTYYSTTYATGQTGNQQTTYAQTNSKKPKSVTDIYLLNQAQSDQQFVATQPFWTHYLINEKGLHNAMCKYTTAYYYTGYYGSTYYTTTTSTDRYLAIVLAANSMNNRTVQHGTTTSQGTWAAATQTAAPQFMYTKQKENECLGDYPEVLQEIWPVLRNVETNFQGYLQPQQTMSYCYFRTTQTLFSCKNHKKAKAIKLTQQTTTTYYQFWTPYNTMTFQETQKLYDFFPSCDMRKDLRLALSVTETVAFSHCCQNPQTYAYAGEKFRRYLYHLYGKCLAVLCGRSVHVDFSSSESQRSRHGSVLSSNFTYHTSDGDALLLL

SEQ ID NO: 362 - protein, 355 aa, Artificial Sequence
  Description of Artificial Sequence Synthetic polypeptide
MDQFPESVTENFEYDDLAEACYIGDIVVFGTTYQSTYYSTTYATGQTGNLQVTFAQTNSKKPKSVTDIYLLNQAQSDQLFVATQPFWTHYLINEKGLHNAMCKYTTAYYYTGYYGSTYYTTTTSTDRYLAIVLAANSMNNRTVQHGTTTSQGVWAAATQTAAPQFMYTKQKENECLGDYPEVLQEIWPVLRNVETNYQGYQQPQQTMSYCYFRITQTLFSCKNHKKAKAIKQIQQTTTTFFQYWTPYNTMTYQETQKLYDFFPSCDMRKDLRLAQSVTETTAFSHCCQNPQIYAYAGEKFRRYLYHLYGKCLAVLCGRSVHVDFSSSESQRSRHGSVLSSNFTYHTSDGDALLLL

SEQ ID NO: 363 - protein, 355 aa, Artificial Sequence
  Description of Artificial Sequence Synthetic polypeptide
MDQFPESVTENFEYDDLAEACYIGDIVVFGTTYQSTYYSTTYATGQTGNLQVTFAQTNSKKPKSVTDIYLQNLAQSDQQYTATQPFWTHYLINEKGLHNAMCKYTTAYYYTGYYGSTYYTTTTSTDRYLAIVLAANSMNNRTVQHGVTTSQGTWAAATQTAAPQFMFTKQKENECLGDYPEVLQEIWPVLRNVETNFQGFLQPQQTMSYCYFRTTQTLFSCKNHKKAKAIKLIQQTTTTFYQYWTPYNVMTFQETQKLYDFFPSCDMRKDLRLALSTTETTAYSHCCQNPQTYAYAGEKFRRYLYHLYGKCLAVLCGRSVHVDFSSSESQRSRHGSVLSSNFTYHTSDGDALLLL

SEQ ID NO: 364 - protein, 355 aa, Artificial Sequence
  Description of Artificial Sequence Synthetic polypeptide
MDQFPESVTENFEYDDLAEACYIGDIVVFGTTYQSTYYSTTYATGQTGNLQVTFAQTNSKKPKSVTDIYQQNQAQSDQQYTATQPYWTHYLINEKGLHNAMCKYTTAYYYTGYYGSTYYTTTTSTDRYLAIVLAANSMNNRTVQHGTTTSQGVWAAATQTAAPQFMYTKQKENECLGDYPEVLQEIWPVLRNVETNFQGFLQPQQTMSYCYFRITQTLFSCKNHKKAKAIKLIQQTTTTFYQFWTPYNTMTFQETLKLYDFFPSCDMRKDLRLAQSTTETTAYSHCCQNPQTYAYAGEKFRRYLYHLYGKCLAVLCGRSVHVDFSSSESQRSRHGSVLSSNFTYHTSDGDALLLL

SEQ ID NO: 365 - protein, 355 aa, Artificial Sequence
  Description of Artificial Sequence Synthetic polypeptide
MDQFPESVTENFEYDDLAEACYIGDIVVFGTTYQSTYYSTTYATGQVGNQQVVFALTNSKKPKSVTDIYQQNLAQSDQQFTATQPYWTHYLINEKGLHNAMCKYTTAYYYTGYYGSTYYTTTTSTDRYLAIVLAANSMNNRTVQHGTTTSQGTWAAATQTAAPQFMFTKQKENECLGDYPEVLQEIWPVLRNVETNYQGYQQPQQTMSYCYYRTTQTLFSCKNHKKAKAIKLIQQTTTTFYQFWTPYNTMTFQETLKLYDFFPSCDMRKDLRLALSVTETVAFSHCCQNPQIYAYAGEKFRRYLYHLYGKCLAVLCGRSVHVDFSSSESQRSRHGSVLSSNFTYHTSDGDALLLL

SEQ ID NO: 366 - protein, 355 aa, Artificial Sequence; identical to SEQ ID NO: 360
  Description of Artificial Sequence Synthetic polypeptide

SEQ ID NO: 367 - protein, 355 aa, Unknown
  Description of Unknown Mammalian CCR3 polypeptide
MTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 368 - protein, 355 aa, Artificial Sequence
  Description of Artificial Sequence Synthetic polypeptide
MTTSLDTVETFGTTSYYDDVGLLCEKADTRALMAQFVPPQYSQTYTTGQQGNTTVTMTQIKYRRLRIMTNIYQQNQAISDQQYQVTQPYWTHYVRGHNWVFGHGMCKQLSGYYHTGQYSETYYTTQQTTDRYLAIVHAVFALRARTTTFGTTTSTVTWGQAVQAAQPEFIFYETEELFEETLCSALYPEDTVYSWRHFHTLRMTIYCQVQPQQVMATCYTGTTKTLLRCPSKKKYKAIRQTYTTMATYYTYWTPYNTATQQSSYQSILFGNDCERSKHLDLTMQTTETTAYSHCCMNPTTYAYVGERFRMYLRHFFHRHLLMHLGRYIPFLPSEKLERTSSVSPSTAEPELSIVF

SEQ ID NO: 369 - protein, 355 aa, Artificial Sequence
  Description of Artificial Sequence Synthetic polypeptide
MTTSLDTVETFGTTSYYDDVGLLCEKADTRALMAQFTPPQYSQTFTTGQQGNTTVTMTQIKYRRLRIMTNIYLQNQAISDQLFQTTQPYWTHYVRGHNWVFGHGMCKQLSGFYHTGQYSETFYTTQQTTDRYLAIVHAVFALRARTTTYGTTTSTTTWGQATQAAQPEFIYYETEELFEETLCSALYPEDTVYSWRHFHTLRMTIYCQVQPQQVMATCYTGTTKTLLRCPSKKKYKAIRQTYTTMATYYTYWTPYNTATQQSSYQSILFGNDCERSKHLDLVMQVTETTAYSHCCMNPVTYAYTGERFRKYLRHFFHRHLLMHLGRYIPFLPSEKLERTSSVSPSTAEPELSIVF

SEQ ID NO: 370 - protein, 355 aa, Artificial Sequence
  Description of Artificial Sequence Synthetic polypeptide
MTTSLDTVETFGTTSYYDDVGLLCEKADTRALMAQFTPPQYSQTYTTGQQGNTTVTMTQIKYRRLRIMTNIYQQNLAISDQQFQTTQPFWTHYVRGHNWVFGHGMCKQQSGFYHTGQYSETFFTTQQTTDRYLAIVHAVFALRARTTTYGTTTSTTTWGQATQAAQPEFIYYETEELFEETLCSALYPEDTVYSWRHFHTLRMTTYCQTQPQQTMATCYTGTTKTLLRCPSKKKYKAIRQTYTTMATYYTYWTPYNTATQQSSYQSILFGNDCERSKHLDLVMQVTETTAYSHCCMNPTTYAFTGERFRKYLRHFFHRHLLMHLGRYIPFLPSEKLERTSSVSPSTAEPELSIVF

SEQ ID NO: 371 - protein, 355 aa, Artificial Sequence
  Description of Artificial Sequence Synthetic polypeptide
MTTSLDTVETFGTTSYYDDVGLLCEKADTRALMAQFTPPQYSQTYTTGQQGNVTVTMTQIKYRRLRIMTNIYLQNQAISDQLFQTTQPFWTHYVRGHNWVFGHGMCKQQSGFYHTGQYSETFYTTQQTTDRYLAIVHAVFALRARTTTYGTTTSTTTWGQAVQAAQPEFTFYETEELFEETLCSALYPEDTVYSWRHFHTLRMTIFCQTQPQQTMATCYTGITKTLLRCPSKKKYKAIRQTYTTMATYYTYWTPYNTATQQSSYQSILFGNDCERSKHLDLTMQTTETTAYSHCCMNPTTYAYTGERFRKYLRHFFHRHLLMHLGRYIPFLPSEKLERTSSVSPSTAEPELSIVF

SEQ ID NO: 372 - protein, 355 aa, Artificial Sequence
  Description of Artificial Sequence Synthetic polypeptide
MTTSLDTVETFGTTSYYDDVGLLCEKADTRALMAQFTPPQYSQTFTTGQQGNTTVTMTQIKYRRLRIMTNIYLQNQAISDQLFQTTQPYWTHYVRGHNWVFGHGMCKQQSGFYHTGQYSETFFTTQQTTDRYLAIVHAVFALRARTTTYGTTTSTTTWGQAVQAAQPEFIFYETEELFEETLCSALYPEDTVYSWRHFHTLRMTTYCQTQPQQTMATCYTGTTKTLLRCPSKKKYEAIRQTYTTMATYYTYWTPYNTATQQSSYQSILFGNDCERSKHLDLTMQVTETIAYSHCCMNPTTYAFTGERFRKYLRHFFHRHLLMHLGRYIPFLPSEKLERTSSVSPSTAEPELSIVF

SEQ ID NO: 373 - protein, 355 aa, Artificial Sequence
  Description of Artificial Sequence Synthetic polypeptide
MTTSLDTVETFGTTSYYDDVGLLCEKADTRALMAQFVPPQYSQTYTTGQQGNTTVTMTQIKYRRLRIMTNIYQQNLAISDQQYQVTQPYWTHYVRGHNWVFGHGMCKQLSGFYHTGQYSETFFTTQQTTDRYLAIVHAVFALRARTTTYGTTTSTTTWGQATQAAQPEFIYYETEELFEETLCSALYPEDTVYSWRHFHTLRMTIFCQTQPQQTMATCYTGTIKTLLRCPSKKKYKAIRQTYTTMATYYTYWTPYNTATQQSSYQSILFGNDCERSKHLDLVMQTTETIAYSHCCMNPTTYAYTGERFRKYLRHFFHRHLLMHLGRYIPFLPSEKLERTSSVSPSTAEPELSIVF

SEQ ID NO: 374 - protein, 355 aa, Artificial Sequence
  Description of Artificial Sequence Synthetic polypeptide
MTTSLDTVETFGTTSYYDDVGLLCEKADTRALMAQFVPPQYSQTFTTGQQGNTTVTMTQIKYRRLRIMTNIYQQNLAISDQQYQVTQPFWIHYVRGHNWVFGHGMCKQLSGYYHTGQYSETFFTTQQTTDRYLAIVHAVFALRARTTTFGTTTSTTTWGQAVQAAQPEFIFYETEELFEETLCSALYPEDTVYSWRHFHTLRMTIYCQVQPQQVMATCYTGTTKTPLRCPSKKKYKAIRQTYTTMATYYTYWTPYNTATQQSSYQSILFGNDCERSKHLDLTMQVTETIAYSHCCMNPTTYAYTGERFRKYLRHFFHRHLLMHLGRYIPFLPSEKLERTSSVSPSTAEPELSIVF

SEQ ID NO: 375 - protein, 355 aa, Artificial Sequence
  Description of Artificial Sequence Synthetic polypeptide
MTTSLDTVETFGTTSYYDDVGLLCEKADTRALMAQFTPPQYSQTYTTGQQGNTVTTMTQIKYRRLRIMTNIYQQNLAISDQQFQTTQPFWTHYVRGHNWVFGHGMCKQLSGYYHTGQYSETFFTTQQTTDRYLAIVHAVFALRARTTTYGTTTSTTTWGQATQAAQPEFIFYETEELFEETLCSALYPEDTVYSWRHFHTLRMTIFCQTQPQQTMATCYTGTTKTLLRCPSKKKYKAIRQTYTTMATYYTYWTPYNTATQQSSYQSILFGNDCERSKHLDLVMLTTEVTAYSHCCMNPTTYAFTGGRFRKYLRHFFHRHLLMHLGRYIPFLPSEKLERTSSVSPSTAEPELSIVF

SEQ ID NO: 376 - protein, 355 aa, Artificial Sequence
  Description of Artificial Sequence Synthetic polypeptide
MTTSLDTVETFGTTSYYDDVGLLCEKADTRALMAQFTPPQYSQTYTTGQQGNTTTTMTQTKYRRLRIMTNIYLQNQATSDQLFQTTQPYWTHYVRGHNWVFGHGMCKQQSGFYHTGQYSETFFTTQQTTDRYLAIVHAVFALRARTTTFGTTTSTTTWGQATQAAQPEYTYYETEELFEETLCSALYPEDTVYSWRHFHTLRMTIFCQVQPQQTMATCYTGTTKTLLRCPSKKKYKAIRQTYTTMATYYTYWTPYNTATQQSSYQSILFGNDCERSKHLDLTMQVTETIAYSHCCMNPTTYAYTGERFRKYLRHFFHRHLLMHLGRYIPFLPSEKLERTSSVSPSTAEPELSIVF

SEQ ID NO: 377 - protein, 355 aa, Artificial Sequence
  Description of Artificial Sequence Synthetic polypeptide
MTTSLDTVETFGTTSYYDDVGLLCEKADTRALMAQFTPPQYSQTYTTGQQGNTTTTMTQIKYRRLRIMTNIYQQNQATSDQQYQTTQPYWTHYVRGHNWVFGHGMCKQQSGFYHTGQYSETYYTTQQTTDRYLAIVHAVFALRARTTTFGTTTSTVTWGQAVQAAQPEFTFYETEELFEETLCSALYPEDTVYSWRHFHTLRMTIFCQTQPQQTMATCYTGTTKTLLRCPSKKKYKAIRQTYTTMATYYTYWTPYNTATQQSSYQSILFGNDCERSKHLDLTMQVTETIAYSHCCMNPTTYAFTGERFRKYLRHFFHRHLLMHLGRYIPFLPSEKLERTSSVSPSTAEPELSIVF

SEQ ID NO: 378 - protein, 355 aa, Artificial Sequence
  Description of Artificial Sequence Synthetic polypeptide
MTTSLDTVETFGTTSYYDDVGLLCEKADTRALMAQFTPPQYSQTYTTGQQGNTVTTMTQIKYRRLRIMTNIYLLNQATSDQQFQVTQPFWIHYVRGHNWVFGHGMCKQQSGFYHTGQYSETFYTTQQTTDRYLAIVHAVFALRARTTTYGTTTSTTTWGQATQAAQPEFIYYETEELFEETLCSALYPEDTVYSWRHFHTLRMTIYCQVQPQQVMATCYTGTTKTLLRCPSKKKYKAIRQTYTTMATYYTYWTPYNTATQQSSYQSILFGNDCERSKHLDLTMQTTETTAYSHCCMNPTTYAYTGERFRKYLRHFFHRHLLMHLGRYIPFLPSEKLERTSSVSPSTAEPELSIVF

SEQ ID NO: 379 - protein, 352 aa, Artificial Sequence
  Description of Artificial Sequence Synthetic polypeptide
MDYQVSSPIYDINYYTSEPCQKINVKQIAARLQPPQYSQTFTFGFTGNMQVTQTQINCKRLKSMTDIYLQNQAISDQFFQQTTPYWAHYAAAQWDFGNTMCQQQTGQYFTGYYSGTYYTTQQTTDRYLAVVHAVFALKARTTTYGTTTSTTTWTTATYASQPGTTYTRSQKEGLHYTCSSHFPYSQYQFWKNFQTLKIVIQGQVQPQQTMTTCYSGIQKTLLRCRNEKKRHRAVRQTYTTMTTYYQYWAPYNTVQQLNTFQEFFGLNNCSSSNRLDQAMQVTETQGMTHCCINPTIYAYVGEKFRNYLLVFFQKHIAKRFCKCCSIFQQEAPERASSVYTRSTGEQEISVGL

SEQ ID NO: 380 - protein, 352 aa, Artificial Sequence
  Description of Artificial Sequence Synthetic polypeptide
MDYQVSSPIYDINYYTSEPCQKINVKQIAARQQPPQYSQTYTFGFTGNMQTTQTQINCKRLKSMTDIYLQNQAISDQYFQQTTPYWAHYAAAQWDFGNTMCQQQTGQYFTGYYSGTYYTTQQTTDRYLAVVHAVFALKARTTTYGTTTSTTTWTTATYASQPGTTYTRSQKEGLHYTCSSHFPYSQYQFWKNFQTLKIVIQGQVQPQQTMTTCYSGIQKTLLRCRNEKKRHRAVRQTYTTMTTYYQYWAPYNTVQQLNTFQEFFGLNNCSSSNRLDQAMQTTETQGMTHCCINPTIYAYTGEKFRNYLLVFFQKHIAKRFCKCCSIFQQEAPERASSVYTRSTGEQEISVGL

SEQ ID NO: 381 - protein, 352 aa, Artificial Sequence
  Description of Artificial Sequence Synthetic polypeptide
MDYQVSSPIYDINYYTSEPCQKINVKQIAARQQPPQYSQTFTFGYTGNMQTTQTQINCKRLKSMTDIYLQNQAISDQYYQQTTPYWAHYAAAQWDFGNTMCQQQTGQYFTGYYSGTYYTTQQTTDRYLAVVHAVFALKARTTTYGTTTSTTTWTTATYASQPGTTYTRSQKEGLHYTCSSHFPYSQYQFWKNFQTLKITIQGQVQPQQVMTTCYSGTQKTLLRCRNEKKRHRAVRQTFTTMTTYYQYWAPYNTVQQLNTFQEFFGLNNCSSSNRLDQAMQVTETQGMTHCCINPTIYAYVGEKFRNYLLVFFQKHIAKRFCKCCSIFQQEAPERASSVYTRSTGEQEISVGL

SEQ ID NO: 382 - protein, 352 aa, Artificial Sequence
  Description of Artificial Sequence Synthetic polypeptide
MDYQVSSPIYDINYYTSEPCQKINVKQIAARQQPPQYSQTFTYGFTGNMQTTQTQINCKRLKSMTDIYLQNQAISDQYYQQTTPYWAHYAAAQWDFGNTMCQQQTGQYFTGYYSGTYYTTQQTTDRYLAVVHAVFALKARTTTYGTTTSTTTWTTATYASQPGTTYTRSQKEGLHYTCSSHFPYSQYQFWKNFQTLKITIQGQVQPQQTMVTCYSGTQKTLLRCRNEKKRHRAVRQTFTTMTTYYQYWAPYNTTQQLNTFQEFFGLNNCSSSNRLDQAMQVTETQGMTHCCTNPTIYAYVGEKFRNYLLVFFQKHIAKRFCKCCSIFQQEAPERASSVYTRSTGEQEISVGL